SEQUENCE LISTING (42 sequences)

SEQ ID NO: 1 - protein, 860 aa, Homo sapiens [sequence omitted]

SEQ ID NO: 2 - protein, 41 aa, Homo sapiens
SVTCKSGDFSCGGRVNRCIPQFWRCDGQVDCDNGSDEQGCP

SEQ ID NO: 3 - protein, 39 aa, Homo sapiens
PKTCSQDEFRCHDGKCISRQFVCDSDRDCLDGSDEASCP

SEQ ID NO: 4 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 19 aa, synthetic construct
MKILILGIFLFLCSTPAWA

SEQ ID NO: 6 - protein, 19 aa, synthetic construct
MGWSCIILFLVATATGVHS

SEQ ID NO: 7 - protein, 294 aa, synthetic construct
  SIGNAL 1..19
MKILILGIFLFLCSTPAWASVTCKSGDFSCGGRVNRCIPQFWRCDGQVDCDNGSDEQGCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 333 aa, synthetic construct
  SIGNAL 1..19
MKILILGIFLFLCSTPAWASVTCKSGDFSCGGRVNRCIPQFWRCDGQVDCDNGSDEQGCPPKTCSQDEFRCHDGKCISRQFVCDSDRDCLDGSDEASCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 294 aa, synthetic construct
  SIGNAL 1..19
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSVTCKSGDFSCGGRVNRCIPQFWRCDGQVDCDNGSDEQGCP

SEQ ID NO: 10 - protein, 333 aa, synthetic construct
  SIGNAL 1..19
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSVTCKSGDFSCGGRVNRCIPQFWRCDGQVDCDNGSDEQGCPPKTCSQDEFRCHDGKCISRQFVCDSDRDCLDGSDEASCP

SEQ ID NO: 11 - protein, 386 aa, synthetic construct
  SIGNAL 1..19
MKILILGIFLFLCSTPAWASVTCKSGDFSCGGRVNRCIPQFWRCDGQVDCDNGSDEQGCPGSGGSSVTCKSGDFSCGGRVNRCIPQFWRCDGQVDCDNGSDEQGCPGGSSGSVTCKSGDFSCGGRVNRCIPQFWRCDGQVDCDNGSDEQGCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 478 aa, synthetic construct
  SIGNAL 1..19
MKILILGIFLFLCSTPAWASVTCKSGDFSCGGRVNRCIPQFWRCDGQVDCDNGSDEQGCPGSGGSSVTCKSGDFSCGGRVNRCIPQFWRCDGQVDCDNGSDEQGCPGGSSGSVTCKSGDFSCGGRVNRCIPQFWRCDGQVDCDNGSDEQGCPGGGGSSVTCKSGDFSCGGRVNRCIPQFWRCDGQVDCDNGSDEQGCPGSGSGSVTCKSGDFSCGGRVNRCIPQFWRCDGQVDCDNGSDEQGCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 380 aa, synthetic construct
  SIGNAL 1..19
MKILILGIFLFLCSTPAWAPKTCSQDEFRCHDGKCISRQFVCDSDRDCLDGSDEASCPGSGGSPKTCSQDEFRCHDGKCISRQFVCDSDRDCLDGSDEASCPGGSSGPKTCSQDEFRCHDGKCISRQFVCDSDRDCLDGSDEASCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 386 aa, synthetic construct
  SIGNAL 1..19
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSVTCKSGDFSCGGRVNRCIPQFWRCDGQVDCDNGSDEQGCPGSGGSSVTCKSGDFSCGGRVNRCIPQFWRCDGQVDCDNGSDEQGCPGGSSGSVTCKSGDFSCGGRVNRCIPQFWRCDGQVDCDNGSDEQGCP

SEQ ID NO: 15 - protein, 478 aa, synthetic construct
  SIGNAL 1..19
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSVTCKSGDFSCGGRVNRCIPQFWRCDGQVDCDNGSDEQGCPGSGGSSVTCKSGDFSCGGRVNRCIPQFWRCDGQVDCDNGSDEQGCPGGSSGSVTCKSGDFSCGGRVNRCIPQFWRCDGQVDCDNGSDEQGCPGGGGSSVTCKSGDFSCGGRVNRCIPQFWRCDGQVDCDNGSDEQGCPGSGSGSVTCKSGDFSCGGRVNRCIPQFWRCDGQVDCDNGSDEQGCP

SEQ ID NO: 16 - protein, 380 aa, synthetic construct
  SIGNAL 1..19
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPKTCSQDEFRCHDGKCISRQFVCDSDRDCLDGSDEASCPGSGGSPKTCSQDEFRCHDGKCISRQFVCDSDRDCLDGSDEASCPGGSSGPKTCSQDEFRCHDGKCISRQFVCDSDRDCLDGSDEASCP

SEQ ID NO: 17 - protein, 333 aa, synthetic construct
  SIGNAL 1..19
MKILILGIFLFLCSTPAWAPKTCSQDEFRCHDGKCISRQFVCDSDRDCLDGSDEASCPGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPKTCSQDEFRCHDGKCISRQFVCDSDRDCLDGSDEASCP

SEQ ID NO: 18 - protein, 333 aa, synthetic construct; identical to SEQ ID NO: 17
  SIGNAL 1..19

SEQ ID NO: 19 - DNA, 906 bp, synthetic construct
  source 1..906: mol_type = other DNA
gctagcgccaccatgaagatcctgatcctgggcatcttcctgtttctgtgcagcacccctgcctgggcaagcgtgacctgcaagagcggcgacttcagctgtggcggcagagtgaacagatgcatcccccagttttggagatgcgacggccaggtggactgcgacaatggctctgacgagcagggctgccccggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgag

SEQ ID NO: 20 - DNA, 1023 bp, synthetic construct
  source 1..1023: mol_type = other DNA
gctagcgccaccatgaagatcctgatcctgggcatcttcctgtttctgtgcagcacccctgcctgggcaagcgtgacctgcaagagcggcgacttcagctgtggcggcagagtgaacagatgcatcccccagttttggagatgcgacggccaggtggactgcgacaatggctctgacgagcagggctgcccccctaagacctgcagccaggacgagttcagatgtcacgatggcaagtgtatctcccggcagttcgtgtgcgacagcgacagagactgcctggacggctctgatgaggcctcctgtcctggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgag

SEQ ID NO: 21 - DNA, 988 bp, synthetic construct
  source 1..988: mol_type = other DNA
  intron 59..140
gctagcgccaccatgggatggagctgtatcatcctcttcttggtagcaacagctacaggtaaggggttaacagtagcaggcttgaggtctggacatatatatgggtgacaatgacatccactttgcctttctctccacaggcgtgcactctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccagcgtgacctgcaagagcggcgacttcagctgtggcggcagagtgaacagatgcatcccccagttttggagatgcgacggccaggtggactgcgacaatggctctgacgagcagggctgcccctgataactcgag

SEQ ID NO: 22 - DNA, 1105 bp, synthetic construct
  source 1..1105: mol_type = other DNA
  intron 59..140
gctagcgccaccatgggatggagctgtatcatcctcttcttggtagcaacagctacaggtaaggggttaacagtagcaggcttgaggtctggacatatatatgggtgacaatgacatccactttgcctttctctccacaggcgtgcactctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccagcgtgacctgcaagagcggcgacttcagctgtggcggcagagtgaacagatgcatcccccagttttggagatgcgacggccaggtggactgcgacaatggctctgacgagcagggctgcccccctaagacctgcagccaggacgagttcagatgtcacgatggcaagtgtatctcccggcagttcgtgtgcgacagcgacagagactgcctggacggctctgatgaggcctcctgtccttgataactcgag

SEQ ID NO: 23 - DNA, 1182 bp, synthetic construct
  source 1..1182: mol_type = other DNA
gctagcgccaccatgaagatcctgatcctgggcatcttcctgtttctgtgcagcacaccagcctgggccagcgtgacatgtaaaagcggcgatttcagctgcggcggcagagtgaacagatgcatccctcagttttggagatgcgacggccaggtggactgcgacaatggctctgatgagcagggctgtcctggcagcggaggatcttctgtgacatgcaagtccggcgacttctcctgtggcggaagggtcaacagatgtattccgcaattttggcgctgtgatggacaggtcgactgtgacaacggaagcgacgaacagggatgtccaggcggcagttctggaagcgtgacctgcaagagcggagacttttcctgcggaggccgggtcaaccgttgcattccacaattttggcgttgcgacggacaagttgattgcgataacggttccgacgagcaaggctgcccaggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgag

SEQ ID NO: 24 - DNA, 1458 bp, synthetic construct
  source 1..1458: mol_type = other DNA
gctagcgccaccatgaagatcctgatcctgggcatcttcctgtttctgtgcagcacaccagcctgggccagcgtgacatgtaaaagcggcgatttcagctgcggcggcagagtgaacagatgcatccctcagttttggagatgcgacggccaggtggactgcgacaatggctctgatgagcagggctgtcctggcagcggaggatcttctgtgacatgcaagtccggcgacttctcctgtggcggaagggtcaacagatgtattccgcaattttggcgctgtgatggacaggtcgactgtgacaacggaagcgacgaacagggatgtccaggcggcagttctggaagcgtgacctgcaagagcggagacttttcctgcggaggccgggtcaaccgttgcattccacaattttggcgttgcgacggacaagttgattgcgataacggttccgacgagcaaggctgcccaggcggaggtggaagttccgtgacttgtaaatctggcgatttttcttgcggcggacgcgtgaaccggtgtatcccacaattttggagatgtgatgggcaagtcgattgtgataatggcagtgacgagcaggggtgccctggatctggttctggatctgtcacctgtaaatccggggactttagttgcggtggaagagtgaatcggtgcattccacagttttggcgctgtgacggccaagtggactgtgataacggatcagatgaacagggctgccccggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgag

SEQ ID NO: 25 - DNA, 1164 bp, synthetic construct
  source 1..1164: mol_type = other DNA
gctagcgccaccatgaagatcctgatcctgggcatcttcctgtttctgtgcagcacccctgcctgggctcctaagacatgtagccaggacgagttcagatgccacgacggcaagtgcatcagccggcagttcgtgtgcgacagcgacagagattgtctggacggcagcgacgaggcctcttgtcctggatctggcggaagcccaaagacctgctctcaggatgagttccggtgtcacgatggaaagtgtatctcccgccagtttgtgtgtgactccgacagggactgcctggatggctccgatgaagcttcttgtccaggcggcagcagcggccctaaaacatgttcccaggatgaatttcggtgccatgacgggaaatgcatctccagacagttcgtctgtgatagcgaccgcgactgcctcgacggatctgatgaggcaagttgtcctggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgag

SEQ ID NO: 26 - DNA, 1264 bp, synthetic construct
  source 1..1264: mol_type = other DNA
  intron 59..140
gctagcgccaccatgggatggagctgtatcatcctcttcttggtagcaacagctacaggtaaggggttaacagtagcaggcttgaggtctggacatatatatgggtgacaatgacatccactttgcctttctctccacaggcgtgcactctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccagcgtgacatgtaaaagcggcgatttcagctgcggcggcagagtgaacagatgcatccctcagttttggagatgcgacggccaggtggactgcgacaatggctctgatgagcagggctgtcctggcagcggaggatcttctgtgacatgcaagtccggcgacttctcctgtggcggaagggtcaacagatgtattccgcaattttggcgctgtgatggacaggtcgactgtgacaacggaagcgacgaacagggatgtccaggcggcagttctggaagcgtgacctgcaagagcggagacttttcctgcggaggccgggtcaaccgttgcattccacaattttggcgttgcgacggacaagttgattgcgataacggttccgacgagcaaggctgcccatgataactcgag

SEQ ID NO: 27 - DNA, 1540 bp, synthetic construct
  source 1..1540: mol_type = other DNA
  intron 59..140
gctagcgccaccatgggatggagctgtatcatcctcttcttggtagcaacagctacaggtaaggggttaacagtagcaggcttgaggtctggacatatatatgggtgacaatgacatccactttgcctttctctccacaggcgtgcactctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccagcgtgacatgtaaaagcggcgatttcagctgcggcggcagagtgaacagatgcatccctcagttttggagatgcgacggccaggtggactgcgacaatggctctgatgagcagggctgtcctggcagcggaggatcttctgtgacatgcaagtccggcgacttctcctgtggcggaagggtcaacagatgtattccgcaattttggcgctgtgatggacaggtcgactgtgacaacggaagcgacgaacagggatgtccaggcggcagttctggaagcgtgacctgcaagagcggagacttttcctgcggaggccgggtcaaccgttgcattccacaattttggcgttgcgacggacaagttgattgcgataacggttccgacgagcaaggctgcccaggcggaggtggaagttccgtgacttgtaaatctggcgatttttcttgcggcggacgcgtgaaccggtgtatcccacaattttggagatgtgatgggcaagtcgattgtgataatggcagtgacgagcaggggtgccctggatctggttctggatctgtcacctgtaaatccggggactttagttgcggtggaagagtgaatcggtgcattccacagttttggcgctgtgacggccaagtggactgtgataacggatcagatgaacagggctgcccctgataactcgag

SEQ ID NO: 28 - DNA, 1246 bp, synthetic construct
  source 1..1246: mol_type = other DNA
  intron 59..140
gctagcgccaccatgggatggagctgtatcatcctcttcttggtagcaacagctacaggtaaggggttaacagtagcaggcttgaggtctggacatatatatgggtgacaatgacatccactttgcctttctctccacaggcgtgcactctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcccctaagacatgtagccaggacgagttcagatgccacgacggcaagtgcatcagccggcagttcgtgtgcgacagcgacagagattgtctggacggcagcgacgaggcctcttgtcctggatctggcggaagcccaaagacctgctctcaggatgagttccggtgtcacgatggaaagtgtatctcccgccagtttgtgtgtgactccgacagggactgcctggatggctccgatgaagcttcttgtccaggcggcagcagcggccctaaaacatgttcccaggatgaatttcggtgccatgacgggaaatgcatctccagacagttcgtctgtgatagcgaccgcgactgcctcgacggatctgatgaggcaagttgtccttgataactcgag

SEQ ID NO: 29 - DNA, 1029 bp, synthetic construct
  source 1..1029: mol_type = other DNA
gctagcgccaccatgaagatcctgatcctgggcatcttcctgtttctgtgcagcacccctgcctgggctcctaaaacatgttcccaggatgaatttcggtgccatgacgggaaatgcatctccagacagttcgtctgtgatagcgaccgcgactgcctcgacggatctgatgaggcaagttgtcctggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcccctaaaacatgttcccaggatgaatttcggtgccatgacgggaaatgcatctccagacagttcgtctgtgatagcgaccgcgactgcctcgacggatctgatgaggcaagttgtccttgataatgataactcgag

SEQ ID NO: 30 - DNA, 1029 bp, synthetic construct; identical to SEQ ID NO: 29
  source 1..1029: mol_type = other DNA

SEQ ID NO: 31 - protein, 41 aa, Homo sapiens
DRCERNEFQCQDGKCISYKWVCDGSAECQDGSDESQETCLS

SEQ ID NO: 32 - protein, 41 aa, Homo sapiens
VTCKSGDFSCGGRVNRCIPQFWRCDGQVDCDNGSDEQGCPP

SEQ ID NO: 33 - protein, 39 aa, Homo sapiens
KTCSQDEFRCHDGKCISRQFVCDSDRDCLDGSDEASCPV

SEQ ID NO: 34 - protein, 41 aa, Homo sapiens
LTCGPASFQCNSSTCIPQLWACDNDPDCEDGSDEWPQRCRG

SEQ ID NO: 35 - protein, 39 aa, Homo sapiens
SPCSAFEFHCLSGECIHSSWRCDGGPDCKDKSDEENCAV

SEQ ID NO: 36 - protein, 39 aa, Homo sapiens
ATCRPDEFQCSDGNCIHGSRQCDREYDCKDMSDEVGCVN

SEQ ID NO: 37 - protein, 40 aa, Homo sapiens
TLCEGPNKFKCHSGECITLDKVCNMARDCRDWSDEPIKEC

SEQ ID NO: 38 - protein, 42 aa, Homo sapiens
VGDRCERNEFQCQDGKCISYKWVCDGSAECQDGSDESQETCL

SEQ ID NO: 39 - protein, 41 aa, Homo sapiens
VLTCGPASFQCNSSTCIPQLWACDNDPDCEDGSDEWPQRCR

SEQ ID NO: 40 - protein, 40 aa, Homo sapiens
DSSPCSAFEFHCLSGECIHSSWRCDGGPDCKDKSDEENCA

SEQ ID NO: 41 - protein, 39 aa, Homo sapiens
VATCRPDEFQCSDGNCIHGSRQCDREYDCKDMSDEVGCV

SEQ ID NO: 42 - protein, 41 aa, Homo sapiens
VTLCEGPNKFKCHSGECITLDKVCNMARDCRDWSDEPIKEC

(19) United States
(12) Patent Application Publication
(10) Pub. No.: US 2026/0217786 A1
(43) Pub. Date: Jul. 30, 2026

(54) DISSOCIABLE ENVELOPE BINDING PROTEINS AND USES THEREOF

(71) Applicant: CSL BEHRING LLC, King of Prussia, PA (US)

(72) Inventors: Andreas GILLE, Pasadena, CA (US); Louis Jerry FABRI, Raleigh, NC (US)

(21) Appl. No.: 19/136,618

(22) PCT Filed: Dec. 7, 2023
(86) PCT No.: PCT/IB2023/062380

§ 371 Date: Jun. 6, 2025

Related U.S. Application Data
(60) Provisional application No. 63/386,516, filed on Dec. 8, 2022.

(51) Int. Cl. C07K 14/705; B01D 15/38; C12N 7/00

(52) U.S. Cl. CPC C07K 14/705; B01D 15/3809; C12N 7/00; C07K 2319/30; C07K 2319/40; C12N 2740/00; C12N 2760/20222; C12N 2760/20251

(57) ABSTRACT

The present disclosure relates generally to envelope-binding proteins and their use in methods of producing and/or purifying an enveloped virus from cell culture.

### RELATED APPLICATION DATA

The present application claims priority from U.S. Patent Application No. 63/386,516 filed 8 Dec. 2022 entitled “Dissociable envelope binding proteins and uses thereof”, the entire contents of which is hereby incorporated by reference.

### SEQUENCE LISTING

The present application is filed together with a Sequence Listing in electronic form. The entire contents of the Sequence Listing is hereby incorporated by reference.

### FIELD

The present disclosure relates generally to envelope-binding proteins and their use in methods of producing and/or purifying an enveloped virus from cell culture.

### BACKGROUND

Retroviruses, e.g., lentiviruses are one of the most studied viral vectors for gene therapy. Retroviruses in general are RNA-based viruses which integrate their genetic information into the target cell chromosomes permanently. The advantages of retroviruses include long-term transgene expression in target cells, a low immunogenic potential, and the ability to transduce into dividing and non-dividing cells.

Lentiviruses are genetically engineered and usually based on human immunodeficiency virus 1 (HIV-1). To increase safety, modern vectors contain only those HIV genes which are necessary for infection and gene delivery, but the genes necessary for replication and virulence factors have been removed. Often, the envelope protein of HIV-1 is exchanged with that of another virus to allow infection of a wide range of target cells, e.g., VSV-G protein from Vesicular stomatitis Indiana virus (VSV).

To produce lentiviruses, cells such as human embryonic kidney cells HEK 293T are transfected with 3-4 plasmids. These include the transfer plasmid with the gene of interest and several packaging plasmids encoding, vesicular stomatitis G protein (VSV-G), and essential viral proteins responsible for gene integration or self-assembly. These plasmids can be transiently transfected into the cells, or a producer cell line is created with stable integration of the plasmids with inducible promoters, in which lentivirus production can be induced.

Once the virus production has been induced, the release of the virus occurs by budding after successful assembly within the cells. The lentivirus is harvested from the producer cells and subsequently purified and concentrated in the downstream process.

However, VSV-G is toxic to the producer and transduced cells when expressed continuously, with the high fusogenic activity causing syncytia formation and cellular apoptosis. This has detrimental effects on the producer cell's integrity and ultimately on virus titers. Several cell lines have been developed using alternative G proteins or inducible expression, however low titres or limited tropism has been shown.

Thus, there is a need in the art for an efficient process for producing lentiviruses in a cell culture system, e.g., for gene therapy.

### SUMMARY

In work leading up to the present invention, the inventors sought to produce a method for producing enveloped viruses, e.g., for gene therapy, at commercial scale and/or suitable for regulatory requirements.

Whilst pseudotyping lentivirus with VSV-G increases the range of target cells and contributes to stabilization of the vector, the toxicity of VSV-G to producer cells limits viral production and negatively impacts large scale manufacturing of enveloped viruses. To address this problem, the inventors determined that they could competitively inhibit the interaction between VSV-G pseudotyped virus and LDLR expressing producer cells by production of an envelope-binding protein comprising an LDLR domain fused to an Fc. The inventors conjugated an Fc region of an immunoglobulin to different domains of the human low-density lipoprotein receptor (LDLR). The inventors found that linking the immunoglobulin Fc region to one or more LDLR cysteine-rich complement-type repeat 2 (CR2) and/or complement-type repeat 3 (CR3) domains generated envelope-binding proteins which can bind to VSV-G. This protein was found to inhibit interaction of VSV-G with LDLR expressed on producer cells and reduce cell death and re-infection of cells during virus production. The envelope-binding proteins can subsequently be dissociated from the VSV-G.

The inventors also recognised difficulties associated with purifying the enveloped virus from the cell culture harvest, e.g., degradation of viral particles not only reduces the amount of intact virus to be recovered, but may also compete with intact particles, inhibiting transduction. Additionally, standard purification techniques using ion exchange requires exposing the cell culture to harsh conditions, such as increased salt concentrations which destabilize viral particles. To address these and other problems, the inventors found that they could use an envelope-binding protein of the disclosure as an affinity capture reagent for purifying enveloped viruses.

Based on the foregoing, the present disclosure provides an envelope-binding protein comprising a LDLR domain and a purification tag. The findings by the inventors also provide the basis for methods of producing and/or purifying enveloped viruses.

For example, the present disclosure provides an envelope-binding protein comprising one or more LDLR domains and a purification tag.

In one example, the purification tag is a protein or peptide tag. In one example, the purification tag is a half-life extender. For example, the protein or peptide tag is selected from the group consisting of an immunoglobulin Fc and albumin. In another example, the protein or peptide tag is selected from the group consisting of a hexahistidine tag, a human influenza hemagglutinin (HA) tag, a FLAG-tag, a calmodulin binding peptide (CBP) tag, a poly-glutamate tag, a polycysteine (Cys) tag, a polyhistidine (His) tag, a Myc-tag, a streptavidin-binding peptide (SBP) tag, a streptavidin (Strep) tag, an avidin tag, a bacteriophage V5 epitope (V5) tag, an isopeptag, a SpyTag, a biotin-carboxyl carrier protein (BCCP) tag, a Halo-tag, a thioredoxin (Trx) tag, small ubiquitin-like molecule (SUMO) tag, and a maltose binding protein (MBP) tag.

In one example, the purification tag is not a glutathione S-transferase (GST) tag.

In one example, the purification tag comprises an immunoglobulin Fc.

In the present disclosure, the Fc is not an antibody. For example, the envelope-binding protein of the disclosure comprises only the Fc region of an antibody or comprises the Fc region and the hinge region but not the C_{H}1, C_{L} and variable domains.

In one example, the present disclosure provides an envelope-binding protein comprising one or more LDLR domains and a region consisting of an immunoglobulin Fc.

In one example, the present disclosure provides an envelope-binding protein consisting of one or more LDLR domains, an immunoglobulin Fc and optionally one or more linkers.

In one example, the purification tag comprises an albumin tag.

In one example, the purification tag comprises a hexahistidine tag.

In one example, the purification tag comprises a human influenza hemagglutinin (HA) tag.

In one example, the purification tag comprises a FLAG-tag.

In one example, the purification tag comprises a calmodulin binding peptide (CBP) tag.

In one example, the purification tag comprises a poly-glutamate tag.

In one example, the purification tag comprises a polycysteine (Cys) tag.

In one example, the purification tag comprises a polyhistidine (His) tag.

In one example, the purification tag comprises a Myc-tag.

In one example, the purification tag comprises a streptavidin-binding peptide (SBP) tag.

In one example, the purification tag comprises a streptavidin (Strep) tag.

In one example, the purification tag comprises an avidin tag.

In one example, the purification tag comprises a bacteriophage V5 epitope (V5) tag.

In one example, the purification tag comprises an isopeptag. For example, a peptide which binds covalently to pilin-C protein.

In one example, the purification tag comprises a SpyTag. For example, a peptide which binds covalently to SpyCatcher protein.

In one example, the purification tag comprises a biotin-carboxyl carrier protein (BCCP) tag.

In one example, the purification tag comprises a Halo-tag. For example, the purification tag comprises a modified haloalkane dehalogenase which covalently binds to synthetic ligands comprising a chloroalkane linker.

In one example, the purification tag comprises a thioredoxin (Trx) tag.

In one example, the purification tag comprises a small ubiquitin-like molecule (SUMO) tag.

In one example, the purification tag comprises a maltose binding protein (MBP) tag.

In one example, the envelope-binding protein specifically binds vesicular stomatitis virus G (VSV-G) pseudotyped virus. In another example, the protein specifically binds VSV-G pseudotyped virus in the presence of calcium. In one example, the envelope-binding protein specifically binds VSV-G pseudotyped virus in the presence of at least about 0.01 mM calcium. For example, the protein binds in the presence of about 0.01 mM or more, or about 0.1 mM or more, or about 1 mM or more, about 10 mM or more, or about 100 mM or more of calcium. In one example the protein binds in the presence of about 0.01 mM or more, or about 0.02 mM or more, or about 0.03 mM or more, or about 0.04 mM or more, or about 0.05 mM or more, or about 0.06 mM or more, or about 0.07 mM or more, or about 0.08 mM or more, or about 0.09 mM or more, or about 0.1 mM or more. In one example the protein binds in the presence of about 0.1 mM or more, or about 0.2 mM or more, or about 0.3 mM or more, or about 0.4 mM or more, or about 0.5 mM or more, or about 0.6 mM or more, or about 0.7 mM or more, or about 0.8 mM or more, or about 0.9 mM or more, or about 1 mM or more. In one example the protein binds in the presence of about 1 mM or more, or about 2 mM or more, or about 3 mM or more, or about 4 mM or more, or about 5 mM or more, or about 6 mM or more, or about 7 mM or more, or about 8 mM or more, or about 9 mM or more, or about 10 mM or more. In one example, the protein dissociates from the vesicular stomatitis virus G (VSV-G) pseudotyped virus in the absence of calcium.

In one example, the protein binds VSV-G at a pH greater than about 6.5. For example, the protein of the disclosure binds VSV-G at a pH of between pH 6.5 and pH 9.0. For example, the protein of the disclosure binds VSV-G at a pH of about pH 6.5, about pH 7.0, about pH 7.5, about pH 8.0, about pH 8.5, or about pH 9.0. In one example, the protein binds VSV-G at a pH of about pH 8.0.

In one example, binding of the protein to VSV-G dissociates at a pH below 6.5. For example, binding of the protein to VSV-G dissociates at or below pH 6.0. For example, binding of the protein to VSV-G dissociates at pH 6.0.

In one example, the protein binds VSV-G expressed as virus-like particles (VLPs) on the surface of a cell at an affinity of between 150 pM and 500 pM. For example, the protein binds VSV-G expressed as VLPs on the surface of a cell at an affinity of between 175 pM and 400 pM. In another example, the protein binds VSV-G expressed as VLPs on the surface of a cell at an affinity of between 200 pM and 320 pM. In one example, the protein binds VSV-G expressed as VLPs on the surface of a cell at an affinity of about 200 pM, or about 210 pM, or about 220 pM, or about 230 pM, or about 240 pM, or about 250 pM, or about 260 pM, or about 270 pM, or about 280 pM, or about 290 pM, or about 300 pM, or about 310 pM, or about 320 pM. In one example, the protein binds VSV-G expressed as VLPs on the surface of a cell at an affinity of about 210 pM, for example, about 213 pM. In another example, the protein binds VSV-G expressed as VLPs on the surface of a cell at an affinity of about 280 pM, for example, about 285 pM. In a further example, the protein binds VSV-G expressed as VLPs on the surface of a cell at an affinity of about 290 pM, for example about 291 pM. In one example, the protein binds VSV-G expressed as VLPs on the surface of a cell at an affinity of about 310 pM, for example, about 312 pM.

In one example, the protein binds recombinant VSV-G at an affinity of between 200 pM and 600 pM. For example, the protein binds recombinant VSV-G at an affinity of about 200 pM, for example, about 219.5 pM. In another example, the protein binds recombinant VSV-G at an affinity of between about 350 pM and about 550 pM. For example, the protein binds recombinant VSV-G at an affinity of about 390, for example, about 391 pM. In another example, the protein binds recombinant VSV-G at an affinity of between about 500 pM and about 550 pM. For example, the protein binds recombinant VSV-G at an affinity of about 540 pM, for example, about 537 pM.

In one example, the protein neutralizes transduction of HEK293 cells with an enveloped virus with an IC₅₀ of 80 μg/mL or less. For example, the protein neutralizes transduction of HEK293 cells with an enveloped virus with an IC₅₀ of about 80 μg/mL, or about 70 μg/mL, or about 60 μg/mL, or about 50 μg/mL, or about 40 μg/mL, or about 30 μg/mL, or about 20 μg/mL, or about 10 μg/mL, or about 5 μg/mL. In one example, the protein neutralizes transduction of HEK293 cells with an enveloped virus with an IC₅₀ of about 20 μg/mL or less, for example, with an IC₅₀ of about 17 μg/mL. In one example, the protein neutralizes transduction of HEK293 cells with an enveloped virus with an IC₅₀ of about 10 μg/mL or less, for example, with an IC₅₀ of about 9.3 μg/mL. In one example, the protein neutralizes transduction of HEK293 cells with an enveloped virus with an IC₅₀ of about 3 μg/mL or less, for example, with an IC₅₀ of between about 2 μg/mL to about 3 μg/mL. For example, with an IC₅₀ of about 2.1 μg/mL, or with an IC₅₀ of about 2.6 μg/mL. In another example, the protein neutralizes transduction of HEK293 cells with an enveloped virus with an IC₅₀ of about 1 μg/mL or less. In one example, for example, the protein neutralizes transduction of HEK293 cells with an enveloped virus with an IC₅₀ of about 0.5 μg/mL or less, for example, with an IC₅₀ of about 0.45 μg/mL or about 0.011 μg/mL.

In one example, the one or more LDLR domains comprise a LDLR extracellular domain. For example, the LDLR extracellular domain comprises a ligand binding domain, an epidermal growth factor (EGF) precursor homology domain, and/or an 0-linked sugar domain. In one example, the LDLR extracellular domain comprises a ligand binding domain. In another example, the LDLR extracellular domain comprises an epidermal growth factor (EGF) precursor homology domain. In a further example, the LDLR extracellular domain comprises an O-linked sugar domain.

In one example, the one or more LDLR domains does not comprise a cytoplasmic and/or transmembrane domain. For example, the one or more LDLR domains does not comprise a cytoplasmic domain. In one example, the one or more LDLR domains does not comprise a transmembrane domain. In a further example, the one or more LDLR domains does not comprise a cytoplasmic domain and a transmembrane domain.

In one example, the ligand binding domain comprises one or more complement-type repeat (CR) domains. For example, the one or more CR domains are a CR1 domain, a CR2 domain, a CR3 domain, a CR4 domain, a CR5 domain, a CR6 domain and/or a CR7 domain.

In one example, the envelope-binding protein comprises two or more LDLR domains directly or indirectly linked to each other.

In one example, the two or more LDLR domains are indirectly linked to each other via a linker. For example, the envelope binding protein comprises a linker positioned between each of the LDLR domains. In one example, the linker is a peptide or a polypeptide. For example, the linker is a peptide linker comprising at least 2 amino acids in length. In one example, the linker comprises between 2 and 70 amino acids.

In one example, the linker is a rigid or a flexible linker.

In one example, the linker is selected from the group consisting of a GS linker, a GSGGS linker, a GGSSG linker, a GGGGS linker, a GSGSG linker, a (Gly) linker, a (Gly)₆ linker, a (GGGS)ₙ linker, wherein n=1, 2, 3 or 4, a (EAAAK)ₙ linker, wherein n=1, 2 or 3, a A(EAAAK)₄ALEA(EAAAK₄)A linker, a AEAAAKEAAAKA linker, a (Ala-Pro)₁ linker, wherein n=10-34, and a PAPAP linker.

In one example, the linker is a flexible linker. In one example, the linker comprises glycine or glycine and serine. In one example, the flexible linker is selected from the group consisting of a GS linker, a GSGGS linker, a GGSSG linker, a GGGGS linker, a GSGSG linker, a (Gly)₈ linker, a (Gly)₆ linker and a (GGGS)ₙ linker, wherein n=1, 2, 3 or 4.

In one example, the linker is a rigid linker. In one example, the rigid linker is selected from the group consisting of a (EAAAK)ₙ linker, wherein n=1, 2 or 3, a A(EAAAK)₄ALEA(EAAAK₄)A linker, a AEAAAKEAAAKA linker, a (Ala-Pro)ₙ linker, wherein n=10-34, and a PAPAP linker.

In one example, the two or more LDLR domains are directly linked to each other, e.g., without an intervening linker.

In one example, the purification tag is directly or indirectly linked to the one or more LDLR domains. For example, the immunoglobulin Fc is directly or indirectly linked to the one or more LDLR domains. Since Fc dimerizes with another Fc, an envelope-binding protein of the disclosure can comprise two Fc regions and at least two LDLR domains (i.e., one or more LDLR domains linked to each Fc domain).

In one example, the purification tag is linked to one LDLR domain. For example, the immunoglobulin Fc is linked to one LDLR domain. Since Fc dimerizes with another Fc, an envelope-binding protein of the disclosure can comprise two Fc regions and two LDLR domains.

In one example, each purification tag of the envelope-binding protein is linked to one LDLR domain. In another example, each purification tag of the envelope-binding protein is linked to two or more LDLR domains. For example, each immunoglobulin Fc of the envelope-binding protein is linked to one LDLR domain. In another example, each immunoglobulin Fc of the envelope-binding protein is linked to two or more LDLR domains.

In one example, the purification tag is indirectly linked to the one or more LDLR domains, e.g., via a linker. For example, the envelope binding protein comprises a linker positioned between the purification tag and the LDLR domain. For example, the immunoglobulin Fc is indirectly linked to the one or more LDLR domains, e.g., via a linker. For example, the envelope binding protein comprises a linker positioned between the Fc and the LDLR domain.

In one example, the linker is a peptide or a polypeptide. For example, the linker is a peptide linker comprising at least 2 amino acids in length. In one example, the linker comprises glycine or glycine and serine. In one example, the linker is selected from the group consisting of a GS linker, a GSGGS linker, a GGSSG linker, a GGGGS linker, and a GSGSG linker.

In one example, the purification tag is directly linked to the one or more LDLR domains, e.g., without an intervening linker. For example, the Fc is directly linked to the one or more LDLR domains, e.g., without an intervening linker.

In one example, the LDLR domains are indirectly or directly linked to the N- and/or C-terminus of the purification tag. For example, the LDLR domains are indirectly or directly linked to the N-terminus of the purification tag. In another example, the LDLR domains are indirectly or directly linked to the C-terminus of the purification tag.

In another example, the LDLR domains are indirectly or directly linked to the N- and C-terminus of the purification tag.

In one example, the envelope-binding protein of the disclosure comprises, in order from N- to C-terminus:
(i) a LDLR domain indirectly or directly linked to the purification tag;
(ii) the purification tag indirectly or directly linked to a LDLR domain; or
(iii) a first LDLR domain, the purification tag, and a second LDLR domain, wherein the first and/or second LDLR domains and the purification tag are indirectly or directly linked.

In one example, the envelope-binding protein of the disclosure comprises, in order from N- to C-terminus:
(i) a LDLR domain indirectly or directly linked to the immunoglobulin Fc;
(ii) the immunoglobulin Fc indirectly or directly linked to a LDLR domain; or
(iii) a first LDLR domain, the immunoglobulin Fc, and a second LDLR domain, wherein the first and/or second LDLR domains and the immunoglobulin Fc are indirectly or directly linked.

In one example, the envelope-binding protein of the disclosure comprises, in order from N- to C-terminus a LDLR domain indirectly or directly linked to the purification tag. For example, the envelope-binding protein of the disclosure comprises, in order from N- to C-terminus a LDLR domain indirectly or directly linked to the immunoglobulin Fc.

In one example, the envelope-binding protein of the disclosure comprises, in order from N- to C-terminus the purification tag indirectly or directly linked to a LDLR domain. For example, the envelope-binding protein of the disclosure comprises, in order from N- to C-terminus the immunoglobulin Fc indirectly or directly linked to a LDLR domain.

In one example, the envelope-binding protein of the disclosure comprises, in order from N- to C-terminus a first LDLR domain, the purification tag and a second LDLR domain, wherein the first and/or second LDLR domains and the purification tag are indirectly or directly linked. For example, the envelope-binding protein of the disclosure comprises, in order from N- to C-terminus a first LDLR domain, the immunoglobulin Fc and a second LDLR domain, wherein the first and/or second LDLR domains and the immunoglobulin Fc are indirectly or directly linked.

In one example, the envelope-binding protein comprises one or more additional LDLR domains indirectly or directly linked to the N- and/or C-terminus. For example, the envelope-binding protein comprises one or more additional LDLR domains indirectly or directly linked to the N-terminus. In another example, the envelope-binding protein comprises one or more additional LDLR domains indirectly or directly linked to the C-terminus. In a further example, the envelope-binding protein comprises one or more additional LDLR domains indirectly or directly linked to the N- and C-terminus.

In one example, the one or more LDLR domains are a complement-type repeat 1 (CR1) LDLR domain, a CR2 LDLR domain, a CR3 LDLR domain, a CR4 LDLR domain, a CR5 LDLR domain, a CR6 LDLR domain and/or a CR7 LDLR domain.

In one example, the one or more LDLR domains are a complement-type repeat 2 (CR2) LDLR domains and/or a CR3 LDLR domains. For example, the one or more LDLR domains are one or more CR2 LDLR domains. In another example, the one or more LDLR domains are one or more CR3 LDLR domains. In a further example, the one or more LDLR domains are one or more CR2 LDLR domains and one or more CR3 LDLR domains.

In one example, the protein comprises five or fewer CR2 LDLR domains; and/or five or fewer CR3 LDLR domains. For example, the protein comprises five, or four, or three, or two, or one, or zero CR2 LDLR domains and/or five, or four, or three, or two, or one, or zero CR3 domains.

In one example, the protein comprises one CR2 LDLR domain.

In one example, the protein comprises two CR2 LDLR domains.

In one example, the protein comprises three CR2 LDR domains.

In one example, the protein comprises four CR2 LDR domains.

In one example, the protein comprise five CR2 LDLR domains.

In one example, the protein comprises one CR3 LDLR domain.

In one example, the protein comprises two CR3 LDLR domains.

In one example, the protein comprises three CR3 LDLR domains.

In one example, the protein comprises four CR3 LDR domains.

In one example, the protein comprise five CR3 LDLR domains.

In one example, the protein comprises one CR2 LDLR domain and one CR3 LDLR domain.

In one example, the envelope-binding protein of the disclosure comprises, in order from N- to C-terminus:
(i) a CR2 LDLR domain indirectly or directly linked to the purification tag;
(ii) a CR3 LDLR domain indirectly or directly linked to the purification tag;
(iii) the purification tag indirectly or directly linked to a CR2 LDLR domain;
(iv) the purification tag indirectly or directly linked to a CR3 LDLR domain;
(v) a first CR2 LDLR domain, the purification tag and a second CR2 LDLR domain, wherein each of the CR2 LDLR domains and the purification tag are indirectly or directly linked;
(vi) a first CR3 LDLR domain, the purification tag and a second CR3 LDLR domain, wherein each of the CR3 LDLR domains and the purification tag are indirectly or directly linked;
(vii) a CR2 LDLR domain, the purification tag and a CR3 LDLR domain, wherein the CR2 and CR3 LDLR domains and the purification tag are indirectly or directly linked; or
(viii) a CR3 LDLR domain, the purification tag and a CR2 LDLR domain, wherein the CR2 and CR3 LDLR domains and the purification tag are indirectly or directly linked.

In one example, the envelope-binding protein of the disclosure comprises, in order from N- to C-terminus a CR2 LDLR domain indirectly or directly linked to the purification tag.

In one example, the envelope-binding protein of the disclosure comprises, in order from N- to C-terminus a CR3 LDLR domain indirectly or directly linked to the purification tag.

In one example, the envelope-binding protein of the disclosure comprises, in order from N- to C-terminus the purification tag indirectly or directly linked to a CR2 LDLR domain.

In one example, the envelope-binding protein of the disclosure comprises, in order from N- to C-terminus the purification tag indirectly or directly linked to a CR3 LDLR domain.

In one example, the envelope-binding protein of the disclosure comprises, in order from N- to C-terminus a first CR2 LDLR domain, the purification tag and a second CR2 LDLR domain, wherein each of the CR2 LDLR domains and the purification tag are indirectly or directly linked.

In one example, the envelope-binding protein of the disclosure comprises, in order from N- to C-terminus a first CR3 LDLR domain, the purification tag and a second CR3 LDLR domain, wherein each of the CR3 LDLR domains and the purification tag are indirectly or directly linked.

In one example, the envelope-binding protein of the disclosure comprises, in order from N- to C-terminus a CR2 LDLR domain, the purification tag and a CR3 LDLR domain, wherein the CR2 and CR3 LDLR domains and the purification tag are indirectly or directly linked.

In one example, the envelope-binding protein of the disclosure comprises, in order from N- to C-terminus a CR3 LDLR domain, the purification tag and a CR2 LDLR domain, wherein the CR2 and CR3 LDLR domains and the purification tag are indirectly or directly linked.

In one example, the envelope-binding protein comprises a CR1 LDLR domain, a CR2 LDLR domain, a CR3 LDLR domain, a CR4 LDLR domain, a CR5 LDLR domain, a CR6 LDLR domain and a CR7 LDLR domain. For example, the envelope-binding protein comprises LDLR domains CR1 to CR7 (i.e., CR1, CR2, CR3, CR4, CR5, CR6 and CR7).

In one example, the envelope-binding protein comprises, in order from N- to C-terminus:
(i) a CR1 LDLR domain, a CR2 LDLR domain, a CR3 LDLR domain, a CR4 LDLR domain, a CR5 LDLR domain, a CR6 LDLR domain and a CR7 LDLR domain indirectly or directly linked to the purification tag; or
(ii) the purification tag indirectly or directly linked to a CR1 LDLR domain, a CR2 LDLR domain, a CR3 LDLR domain, a CR4 LDLR domain, a CR5 LDLR domain, a CR6 LDLR domain and a CR7 LDLR domain.

In one example, the envelope-binding protein comprises, in order from N- to C-terminus a CR1 LDLR domain, a CR2 LDLR domain, a CR3 LDLR domain, a CR4 LDLR domain, a CR5 LDLR domain, a CR6 LDLR domain and a CR7 LDLR domain indirectly or directly linked to the purification tag. For example, the envelope-binding protein comprises, in order from N- to C-terminus LDLR domains CR1 to CR7 indirectly or directly linked to the purification tag.

In one example, the envelope-binding protein comprises, in order from N- to C-terminus the purification tag indirectly or directly linked to a CR1 LDLR domain, a CR2 LDLR domain, a CR3 LDLR domain, a CR4 LDLR domain, a CR5 LDLR domain, a CR6 LDLR domain and a CR7 LDLR domain. For example, the envelope-binding protein comprises, in order from N- to C-terminus the purification tag indirectly or directly linked to LDLR domains CR1 to CR7.

In one example, the envelope-binding protein of the disclosure comprises, in order from N- to C-terminus:
(i) a CR2 or CR3 LDLR domain indirectly or directly linked to the immunoglobulin Fc;
(ii) the immunoglobulin Fc indirectly or directly linked to a CR2 or CR3 LDLR domain; or
(iii) a CR2 or CR3 LDLR domain, the immunoglobulin Fc and a second CR2 or CR3 LDLR domain, wherein the CR2 and/or CR3 LDLR domains and the immunoglobulin Fc are indirectly or directly linked.

In one example, the envelope-binding protein of the disclosure comprises, in order from N- to C-terminus a CR2 or CR3 LDLR domain indirectly or directly linked to the immunoglobulin Fc.

In one example, the envelope-binding protein of the disclosure comprises, in order from N- to C-terminus the immunoglobulin Fc indirectly or directly linked to a CR2 or CR3 LDLR domain.

In one example, the envelope-binding protein of the disclosure comprises, in order from N- to C-terminus a CR2 or CR3 LDLR domain, the immunoglobulin Fc and a second CR2 or CR3 LDLR domain, wherein the CR2 and/or CR3 LDLR domains and the immunoglobulin Fc are indirectly or directly linked.

In one example, the envelope-binding protein of the disclosure comprises, in order from N- to C-terminus:
(i) a CR2 LDLR domain indirectly or directly linked to the immunoglobulin Fc;
(ii) a CR3 LDLR domain indirectly or directly linked to the immunoglobulin Fc;
(iii) the immunoglobulin Fc indirectly or directly linked to a CR2 LDLR domain;
(iv) the immunoglobulin Fc indirectly or directly linked to a CR3 LDLR domain;
(v) a first CR2 LDLR domain, the immunoglobulin Fc and a second CR2 LDLR domain, wherein each of the CR2 LDLR domains and the immunoglobulin Fc are indirectly or directly linked;
(vi) a first CR3 LDLR domain, the immunoglobulin Fc and a second CR3 LDLR domain, wherein each of the CR3 LDLR domains and the immunoglobulin Fc are indirectly or directly linked;
(vii) a CR2 LDLR domain, the immunoglobulin Fc and a CR3 LDLR domain, wherein the CR2 and CR3 LDLR domains and the immunoglobulin Fc are indirectly or directly linked; or
(viii) a CR3 LDLR domain, the immunoglobulin Fc and a CR2 LDLR domain, wherein the CR2 and CR3 LDLR domains and the immunoglobulin Fc are indirectly or directly linked.

In one example, the envelope-binding protein of the disclosure comprises, in order from N- to C-terminus a CR2 LDLR domain indirectly or directly linked to the immunoglobulin Fc.

In one example, the envelope-binding protein of the disclosure comprises, in order from N- to C-terminus a CR3 LDLR domain indirectly or directly linked to the immunoglobulin Fc.

In one example, the envelope-binding protein of the disclosure comprises, in order from N- to C-terminus the immunoglobulin Fc indirectly or directly linked to a CR2 LDLR domain.

In one example, the envelope-binding protein of the disclosure comprises, in order from N- to C-terminus the immunoglobulin Fc indirectly or directly linked to a CR3 LDLR domain.

In one example, the envelope-binding protein of the disclosure comprises, in order from N- to C-terminus a first CR2 LDLR domain, the immunoglobulin Fc and a second CR2 LDLR domain, wherein each of the CR2 LDLR domains and the immunoglobulin Fc are indirectly or directly linked.

In one example, the envelope-binding protein of the disclosure comprises, in order from N- to C-terminus a first CR3 LDLR domain, the immunoglobulin Fc and a second CR3 LDLR domain, wherein each of the CR3 LDLR domains and the immunoglobulin Fc are indirectly or directly linked.

In one example, the envelope-binding protein of the disclosure comprises, in order from N- to C-terminus a CR2 LDLR domain, the immunoglobulin Fc and a CR3 LDLR domain, wherein the CR2 and CR3 LDLR domains and the immunoglobulin Fc are indirectly or directly linked.

In one example, the envelope-binding protein of the disclosure comprises, in order from N- to C-terminus a CR3 LDLR domain, the immunoglobulin Fc and a CR2 LDLR domain, wherein the CR2 and CR3 LDLR domains and the immunoglobulin Fc are indirectly or directly linked.

In one example, the protein comprises one or more additional CR2 and/or CR3 LDLR domains indirectly or directly linked to the N- and/or C-terminus. For example, the protein comprises one or more additional CR2 LDLR domains indirectly or directly linked to the N-terminus. In another example, the protein comprises one or more additional CR2 LDLR domains indirectly or directly linked to the C-terminus. In a further example, the protein comprises one or more additional CR2 LDLR domains indirectly or directly linked to the N-terminus and one or more additional CR2 LDLR domains indirectly or directly linked to the C-terminus. For example, the protein comprises one or more additional CR3 LDLR domains indirectly or directly linked to the N-terminus. In another example, the protein comprises one or more additional CR3 LDLR domains indirectly or directly linked to the C-terminus. In a further example, the protein comprises one or more additional CR3 LDLR domains indirectly or directly linked to the N-terminus and one or more additional CR3 LDLR domains indirectly or directly linked to the C-terminus. For example, the protein comprises one or more additional CR2 LDLR domains and one or more additional CR3 LDLR domains indirectly or directly linked to the N-terminus. In another example, the protein comprises one or more additional CR2 LDLR domains and one or more additional CR3 LDLR domains indirectly or directly linked to the C-terminus. In a further example, the protein comprises one or more additional CR2 LDLR domains indirectly or directly linked to the N-terminus and one or more additional CR3 LDLR domains indirectly or directly linked to the C-terminus. In a further example, the protein comprises one or more additional CR3 LDLR domains indirectly or directly linked to the N-terminus and one or more additional CR2 LDLR domains indirectly or directly linked to the C-terminus. In a further example, the protein comprises one or more additional CR2 LDLR domains and one or more additional CR3 LDLR domains indirectly or directly linked to the N-terminus and one or more additional CR2 LDLR domains indirectly or directly linked to the C-terminus. In a further example, the protein comprises one or more additional CR2 LDLR domains indirectly or directly linked to the N-terminus and one or more additional CR2 LDLR domains and one or more additional CR3 LDLR domains indirectly or directly linked to the C-terminus. In a further example, the protein comprises one or more additional CR2 LDLR domains and one or more additional CR3 LDLR domains indirectly or directly linked to the N-terminus and one or more additional CR2 LDLR domains and one or more additional CR3 LDLR domains indirectly or directly linked to the C-terminus.

In one example, the envelope-binding protein of the disclosure comprises, in order from N- to C-terminus:
(i) a CR2 LDLR domain indirectly or directly linked to the immunoglobulin Fc;
(ii) a CR2 LDLR domain, a CR3 LDLR domain and the immunoglobulin Fc, wherein the CR2 and CR3 LDLR domains and the immunoglobulin Fc are indirectly or directly linked;
(iii) the immunoglobulin Fc indirectly or directly linked to a CR2 LDLR domain;
(iv) the immunoglobulin Fc, a CR2 LDLR domain, a CR3 LDLR domain, wherein the immunoglobulin Fc and the CR2 and CR3 LDLR domains are indirectly or directly linked;
(v) a first CR2 LDLR domain, a second CR2 LDLR domain, a third CR2 LDLR domain and the immunoglobulin Fc, wherein the CR2 LDLR domains and the immunoglobulin are indirectly or directly linked;
(vi) a first CR2 LDLR domain, a second CR2 LDLR domain, a third CR2 LDLR domain, a fourth CR2 LDLR domain, a fifth CR2 LDLR domain and the immunoglobulin Fc, wherein the CR2 LDLR domains and the immunoglobulin are indirectly or directly linked;
(vii) a first CR3 LDLR domain, a second CR3 LDLR domain, a third CR3 LDLR domain and the immunoglobulin Fc, wherein the CR3 LDLR domains and the immunoglobulin are indirectly or directly linked;
(viii) the immunoglobulin Fc, a first CR2 LDLR domain, a second CR2 LDLR domain, and a third CR2 LDLR domain, wherein the immunoglobulin and the CR2 LDLR domains are indirectly or directly linked;
(ix) the immunoglobulin Fc, a first CR2 LDLR domain, a second CR2 LDLR domain, a third CR2 LDLR domain, a fourth CR2 LDLR domain and a fifth CR2 LDLR domain, wherein the immunoglobulin and the CR2 LDLR domains are indirectly or directly linked;
(x) the immunoglobulin Fc, a first CR3 LDLR domain, a second CR3 LDLR domain, and a third CR3 LDLR domain, wherein the immunoglobulin and the CR3 LDLR domains are indirectly or directly linked;
(xi) a first CR2 LDLR domain, the immunoglobulin Fc, a second CR2 LDLR domain, wherein the immunoglobulin and the CR2 LDLR domains are indirectly or directly linked; or
(xii) a first CR3 LDLR domain, the immunoglobulin Fc, a second CR3 LDLR domain, wherein the immunoglobulin and the CR3 LDLR domains are indirectly or directly linked.

In one example, the envelope-binding protein of the disclosure comprises, in order from N- to C-terminus a CR2 LDLR domain indirectly or directly linked to the immunoglobulin Fc.

In one example, the envelope-binding protein of the disclosure comprises, in order from N- to C-terminus a CR2 LDLR domain, a CR3 LDLR domain and the immunoglobulin Fc, wherein the CR2 and CR3 LDLR domains and the immunoglobulin Fc are indirectly or directly linked.

In one example, the envelope-binding protein of the disclosure comprises, in order from N- to C-terminus the immunoglobulin Fc indirectly or directly linked to a CR2 LDLR domain.

In one example, the envelope-binding protein of the disclosure comprises, in order from N- to C-terminus the immunoglobulin Fc, a CR2 LDLR domain, a CR3 LDLR domain, wherein the immunoglobulin Fc and the CR2 and CR3 LDLR domains are indirectly or directly linked.

In one example, the envelope-binding protein of the disclosure comprises, in order from N- to C-terminus a first CR2 LDLR domain, a second CR2 LDLR domain, a third CR2 LDLR domain and the immunoglobulin Fc, wherein the CR2 LDLR domains and the immunoglobulin are indirectly or directly linked.

In one example, the envelope-binding protein of the disclosure comprises, in order from N- to C-terminus a first CR2 LDLR domain, a second CR2 LDLR domain, a third CR2 LDLR domain, a fourth CR2 LDLR domain, a fifth CR2 LDLR domain and the immunoglobulin Fc, wherein the CR2 LDLR domains and the immunoglobulin are indirectly or directly linked.

In one example, the envelope-binding protein of the disclosure comprises, in order from N- to C-terminus a first CR3 LDLR domain, a second CR3 LDLR domain, a third CR3 LDLR domain and the immunoglobulin Fc, wherein the CR3 LDLR domains and the immunoglobulin are indirectly or directly linked.

In one example, the envelope-binding protein of the disclosure comprises, in order from N- to C-terminus the immunoglobulin Fc, a first CR2 LDLR domain, a second CR2 LDLR domain, and a third CR2 LDLR domain, wherein the immunoglobulin and the CR2 LDLR domains are indirectly or directly linked.

In one example, the envelope-binding protein of the disclosure comprises, in order from N- to C-terminus the immunoglobulin Fc, a first CR2 LDLR domain, a second CR2 LDLR domain, a third CR2 LDLR domain, a fourth CR2 LDLR domain and a fifth CR2 LDLR domain, wherein the immunoglobulin and the CR2 LDLR domains are indirectly or directly linked.

In one example, the envelope-binding protein of the disclosure comprises, in order from N- to C-terminus the immunoglobulin Fc, a first CR3 LDLR domain, a second CR3 LDLR domain, and a third CR3 LDLR domain, wherein the immunoglobulin and the CR3 LDLR domains are indirectly or directly linked.

In one example, the envelope-binding protein of the disclosure comprises, in order from N- to C-terminus a first CR2 LDLR domain, the immunoglobulin Fc, a second CR2 LDLR domain, wherein the immunoglobulin and the CR2 LDLR domains are indirectly or directly linked.

In one example, the envelope-binding protein of the disclosure comprises, in order from N- to C-terminus a first CR3 LDLR domain, the immunoglobulin Fc, a second CR3 LDLR domain, wherein the immunoglobulin and the CR3 LDLR domains are indirectly or directly linked.

LDLR domains CR1 to CR7 may be defined with reference to human LDLR (as set forth in SEQ ID NO: 1). In one example, the one or more LDLR domains comprises one or more LDLR domains of a sequence set forth in SEQ ID NO: 1.

The skilled person will recognize that some variation around the exact residues included in the LDLR domains CR1 to CR7 is permitted without affecting the function of the protein of the disclosure as is demonstrated by Nikolic, J., Nat Commun 9, 1029 (2018) and Kim and Bezprozvanny, Int. J. Mol. Sci. 22(9), 5030 (2021). For example, the amino acid residue may deviate by 1 or 2 or 3 or 4 or 5 amino acids to the C-terminus and/or the N-terminus of the stated amino acid position.

For example, the one or more LDLR domains comprise a sequence set forth within amino acids 23 (±1 to 5 amino acids) to 313 (±1 to 5 amino acids) of SEQ ID NO: 1. For example, a CR1 LDLR domain comprises amino acids 23 (±1 to 5 amino acids) to 64 (±1 to 5 amino acids) of SEQ ID NO: 1, a CR2 LDLR domain comprises amino acids 65 (±1 to 5 amino acids) to 105 (±1 to 5 amino acids) of SEQ ID NO: 1, a CR3 LDLR domain comprises amino acid residues 106 (±1 to 5 amino acids) to 144 (±1 to 5 amino acids) of SEQ ID NO: 1, a CR4 LDLR domain comprises amino acids 145 (±1 to 5 amino acids) to 185 (±1 to 5 amino acids) of SEQ ID NO: 1, a CR5 LDLR domain comprises amino acids 193 (±1 to 5 amino acids) to 232 (±1 to 5 amino acids) of SEQ ID NO: 1, a CR6 LDLR domain comprises amino acids 233 (±1 to 5 amino acids) to 271 (±1 to 5 amino acids) of SEQ ID NO: 1 and a CR7 LDLR domain comprises amino acids 273 (±1 to 5 amino acids) to 313 (±1 to 5 amino acids) of SEQ ID NO: 1.

In one example, the one or more LDLR domains comprise a sequence set forth within amino acids 23 (±1 to 5 amino acids) to 313 (±1 to 5 amino acids) of SEQ ID NO: 1. For example, a CR1 LDLR domain comprises amino acids 23 (±1 to 5 amino acids) to 64 (±1 to 5 amino acids) of SEQ ID NO: 1. In another example, a CR2 LDLR domain comprises amino acids 65 (±1 to 5 amino acids) to 105 (±1 to 5 amino acids) of SEQ ID NO: 1. In a further example, a CR3 LDLR domain comprises amino acid residues 106 (±1 to 5 amino acids) to 144 (±1 to 5 amino acids) of SEQ ID NO: 1. In one example, a CR4 LDLR domain comprises amino acids 145 (±1 to 5 amino acids) to 185 (±1 to 5 amino acids) of SEQ ID NO: 1. In another example, a CR5 LDLR domain comprises amino acids 193 (±1 to 5 amino acids) to 232 (±1 to 5 amino acids) of SEQ ID NO: 1. In a further example, a CR6 LDLR domain comprises amino acids 233 (±1 to 5 amino acids) to 271 (±1 to 5 amino acids) of SEQ ID NO: 1. In one example, a CR7 LDLR domain comprises amino acids 273 (±1 to 5 amino acids) to 313 (±1 to 5 amino acids) of SEQ ID NO: 1.

In one example, the one or more LDLR domains comprise a sequence set forth within amino acids 23 to 313 of SEQ ID NO: 1. For example, a CR1 LDLR domain comprises amino acids 23 to 64 of SEQ ID NO: 1, a CR2 LDLR domain comprises amino acids 65 to 105 of SEQ ID NO: 1, a CR3 LDLR domain comprises amino acid residues 106 to 144 of SEQ ID NO: 1, a CR4 LDLR domain comprises amino acids 145 to 185 of SEQ ID NO: 1, a CR5 LDLR domain comprises amino acids 193 to 232 of SEQ ID NO: 1, a CR6 LDLR domain comprises amino acids 233 to 271 of SEQ ID NO: 1 and a CR7 LDLR domain comprises amino acids 273 to 313 of SEQ ID NO: 1.

In one example, a CR1 LDLR domain comprises amino acids 23 to 64 of SEQ ID NO: 1. For example, the CR1 LDLR domain comprises a sequence set forth in SEQ ID NO: 38,

In one example, a CR2 LDLR domain comprises amino acids 65 to 105 of SEQ ID NO: 1. For example, the CR2 LDLR domain comprises a sequence set forth in SEQ ID NO: 2.

In one example, a CR3 LDLR domain comprises amino acid residues 106 to 144 of SEQ ID NO: 1. For example, the CR3 LDLR domain comprises a sequence set forth in SEQ ID NO: 3.

In one example, a CR4 LDLR domain comprises amino acids 145 to 185 of SEQ ID NO: 1. For example, the CR4 LDLR domain comprises a sequence set forth in SEQ ID NO: 39.

In one example, a CR5 LDLR domain comprises amino acids 193 to 232 of SEQ ID NO: 1. For example, the CR5 LDLR domain comprises a sequence set forth in SEQ ID NO: 40.

In one example, a CR6 LDLR domain comprises amino acids 233 to 271 of SEQ ID NO: 1. For example, the CR6 LDLR domain comprises a sequence set forth in SEQ ID NO: 41.

In one example, a CR7 LDLR domain comprises amino acids 273 to 313 of SEQ ID NO: 1. For example, the CR7 LDLR domain comprises a sequence set forth in SEQ ID NO: 42.

In one example, the one or more LDLR domains comprise a sequence set forth within amino acids 25 to 313 of SEQ ID NO: 1. For example, a CR1 LDLR domain comprises amino acids 25 to 65 of SEQ ID NO: 1, a CR2 LDLR domain comprises amino acids 66 to 106 of SEQ ID NO: 1, a CR3 LDLR domain comprises amino acid residues 107 to 145 of SEQ ID NO: 1, a CR4 LDLR domain comprises amino acids 146 to 186 of SEQ ID NO: 1, a CR5 LDLR domain comprises amino acids 195 to 233 of SEQ ID NO: 1, a CR6 LDLR domain comprises amino acids 234 to 272 of SEQ ID NO: 1 and a CR7 LDLR domain comprises amino acids 274 to 313 of SEQ ID NO: 1.

In one example, a CR1 LDLR domain comprises amino acids 25 to 65 of SEQ ID NO: 1. For example, the CR1 LDLR domain comprises a sequence set forth in SEQ ID NO: 31.

In one example, a CR2 LDLR domain comprises amino acids 66 to 106 of SEQ ID NO: 1. For example, the CR2 LDLR domain comprises a sequence set forth in SEQ ID NO: 32.

In one example, a CR3 LDLR domain comprises amino acid residues 107 to 145 of SEQ ID NO: 1. For example, the CR3 LDLR domain comprises a sequence set forth in SEQ ID NO: 33.

In one example, a CR4 LDLR domain comprises amino acids 146 to 186 of SEQ ID NO: 1. For example, the CR4 LDLR domain comprises a sequence set forth in SEQ ID NO: 34.

In one example, a CR5 LDLR domain comprises amino acids 195 to 233 of SEQ ID NO: 1. For example, the CR5 LDLR domain comprises a sequence set forth in SEQ ID NO: 35.

In one example, a CR6 LDLR domain comprises amino acids 234 to 272 of SEQ ID NO: 1. For example, the CR6 LDLR domain comprises a sequence set forth in SEQ ID NO: 36.

In one example, a CR7 LDLR domain comprises amino acids 274 to 313 of SEQ ID NO: 1. For example, the CR7 LDLR domain comprises a sequence set forth in SEQ ID NO: 37.

In one example, the one or more LDLR domains comprises a sequence set forth within amino acids 65 (±1 to 5 amino acids) to 144 (±1 to 5 amino acids) of SEQ ID NO: 1. For example, the LDLR domains comprise a sequence set forth in amino acids 65 to 144 of SEQ ID NO: 1. In another example, the LDLR domains comprise a sequence set forth in amino acids 66 to 145 of SEQ ID NO: 1.

In one example, the LDLR domain comprises a sequence set forth in amino acids 23 to 313 of SEQ ID NO: 1. In another example, the LDLR domain comprises one or more sequence set forth in any one of SEQ ID Nos: 2, 3 and 38-42.

In one example, the LDLR domain comprises a sequence set forth in amino acids 25 to 313 of SEQ ID NO: 1. In another example, the LDLR domain comprises one or more sequences set forth in any one of SEQ ID NOs: 31 to 37.

In one example, the immunoglobulin Fc is from IgG. For example, the Fc is from human IgG. In one example, the Fc is from IgG1. For example, the Fc is from human IgG1. An exemplary Fc of the disclosure is an IgG1 Fc comprising a sequence set forth in SEQ ID NO: 4.

In one example, the envelope-binding protein of the disclosure comprises, or consists of, in order from N- to C-terminus:
(i) a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4;
(ii) a CR2 LDLR domain comprising amino acids 65 (±1 to 5 amino acids) to 105 (±1 to 5 amino acids) of SEQ ID NO: 1, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4;
(iii) the immunoglobulin Fc comprising a sequence set forth in SEQ ID NO: 4, a GS linker and a CR2 LDLR domain comprising amino acids 65 (±1 to 5 amino acids) to 105 (±1 to 5 amino acids) of SEQ ID NO: 1;
(iv) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1 and a CR3 LDLR domain comprising amino acid residues 106 (±1 to 5 amino acids) to 144 (±1 to 5 amino acids) of SEQ ID NO: 1;
(v) a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4;
(vi) a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker, a CR2 LDLR domain comprising amino acids 65 (±1 to 5 amino acids) to 105 (±1 to 5 amino acids) of SEQ ID NO: 1 and the immunoglobulin Fc comprising a sequence set forth in SEQ ID NO: 4;
(vii) a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4;
(viii) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker and a CR2 LDLR domain comprising amino acids 65 (±1 to 5 amino acids) to 105 (±1 to 5 amino acids) of SEQ ID NO: 1;
(ix) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker and a CR2 LDLR domain comprising amino acids 65 (±1 to 5 amino acids) to 105 (±1 to 5 amino acids) of SEQ ID NO: 1;
(x) the immunoglobulin Fc comprising a sequence set forth in SEQ ID NO: 4, a GS linker, a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, and a CR3 LDLR domain comprising amino acid residues 106 (±1 to 5 amino acids) to 144 (±1 to 5 amino acids) of SEQ ID NO: 1;
(xi) a CR2 LDLR domain comprising amino acids 65 (±1 to 5 amino acids) to 105 (±1 to 5 amino acids) of SEQ ID NO: 1, a GS linker, the immunoglobulin Fc comprising a sequence set forth in SEQ ID NO: 4, a GS linker, and a CR2 LDLR domain comprising amino acids 65 (±1 to 5 amino acids) to 105 (±1 to 5 amino acids) of SEQ ID NO: 1; or
(xii) a CR3 LDLR domain comprising amino acid residues 106 (±1 to 5 amino acids) to 144 (±1 to 5 amino acids) of SEQ ID NO: 1, a GS linker, the immunoglobulin Fc comprising a sequence set forth in SEQ ID NO: 4, a GS linker, and a CR3 LDLR domain comprising amino acid residues 106 (±1 to 5 amino acids) to 144 (±1 to 5 amino acids) of SEQ ID NO: 1.

In one example, the envelope-binding protein of the disclosure comprises, or consists of, in order from N- to C-terminus a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4.

In one example, the envelope-binding protein of the disclosure comprises, or consists of, in order from N- to C-terminus a CR2 LDLR domain comprising amino acids 65 (±1 to 5 amino acids) to 105 (±1 to 5 amino acids) of SEQ ID NO: 1, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4.

In one example, the envelope-binding protein of the disclosure comprises, or consists of, in order from N- to C-terminus the immunoglobulin Fc comprising a sequence set forth in SEQ ID NO: 4, a GS linker and a CR2 LDLR domain comprising amino acids 65 (±1 to 5 amino acids) to 105 (±1 to 5 amino acids) of SEQ ID NO: 1.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1 and a CR3 LDLR domain comprising amino acid residues 106 (±1 to 5 amino acids) to 144 (±1 to 5 amino acids) of SEQ ID NO: 1.

In one example, the envelope-binding protein of the disclosure comprises, or consists of, in order from N- to C-terminus a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4.

In one example, the envelope-binding protein of the disclosure comprises, or consists of, in order from N- to C-terminus a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker, a CR2 LDLR domain comprising amino acids 65 (±1 to 5 amino acids) to 105 (±1 to 5 amino acids) of SEQ ID NO: 1 and the immunoglobulin Fc comprising a sequence set forth in SEQ ID NO: 4.

In one example, the envelope-binding protein of the disclosure comprises, or consists of, in order from N- to C-terminus a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker and a CR2 LDLR domain comprising amino acids 65 (±1 to 5 amino acids) to 105 (±1 to 5 amino acids) of SEQ ID NO: 1.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker and a CR2 LDLR domain comprising amino acids 65 (±1 to 5 amino acids) to 105 (±1 to 5 amino acids) of SEQ ID NO: 1.

In one example, the envelope-binding protein of the disclosure comprises, or consists of, in order from N- to C-terminus the immunoglobulin Fc comprising a sequence set forth in SEQ ID NO: 4, a GS linker, a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, and a CR3 LDLR domain comprising amino acid residues 106 (±1 to 5 amino acids) to 144 (±1 to 5 amino acids) of SEQ ID NO: 1.

In one example, the envelope-binding protein of the disclosure comprises, or consists of, in order from N- to C-terminus a CR2 LDLR domain comprising amino acids 65 (±1 to 5 amino acids) to 105 (±1 to 5 amino acids) of SEQ ID NO: 1, a GS linker, the immunoglobulin Fc comprising a sequence set forth in SEQ ID NO: 4, a GS linker, and a CR2 LDLR domain comprising amino acids 65 (±1 to 5 amino acids) to 105 (±1 to 5 amino acids) of SEQ ID NO: 1.

In one example, the envelope-binding protein of the disclosure comprises, or consists of, in order from N- to C-terminus a CR3 LDLR domain comprising amino acid residues 106 (±1 to 5 amino acids) to 144 (±1 to 5 amino acids) of SEQ ID NO: 1, a GS linker, the immunoglobulin Fc comprising a sequence set forth in SEQ ID NO: 4, a GS linker, and a CR3 LDLR domain comprising amino acid residues 106 (±1 to 5 amino acids) to 144 (±1 to 5 amino acids) of SEQ ID NO: 1.

In one example, the envelope-binding protein of the disclosure comprises, or consists of, in order from N- to C-terminus:
(i) a CR2 LDLR domain comprising a sequence set forth in SEQ ID NO: 2, a GS linker and the immunoglobulin Fc comprising a sequence set forth in SEQ ID NO: 4;
(ii) a CR2 LDLR domain comprising a sequence set forth in SEQ ID NO: 2, a CR3 LDLR domain comprising a sequence set forth in SEQ ID NO: 3, a GS linker and the immunoglobulin Fc comprising a sequence set forth in SEQ ID NO: 4;
(iii) the immunoglobulin Fc comprising a sequence set forth in SEQ ID NO: 4, a GS linker and a CR2 LDLR domain comprising a sequence set forth in SEQ ID NO: 2;
(iv) the immunoglobulin Fc comprising a sequence set forth in SEQ ID NO: 4, a GS linker, a CR2 LDLR domain comprising a sequence set forth in SEQ ID NO: 2 and a CR3 LDLR domain comprising a sequence set forth in SEQ ID NO: 3;
(v) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GS linker and the immunoglobulin Fc comprising a sequence set forth in SEQ ID NO: 4;
(vi) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker, a CR2 LDLR domain comprising a sequence set forth in SEQ ID NO: 2 and the immunoglobulin Fc comprising a sequence set forth in SEQ ID NO: 4;
(vii) a CR3 LDLR domain comprising a sequence set forth in SEQ ID NO: 3, a GSGGS linker, a CR3 LDLR domain comprising a sequence set forth in SEQ ID NO: 3, a GGSSG linker, a CR3 LDLR domain comprising a sequence set forth in SEQ ID NO: 3, a GS linker and the immunoglobulin Fc comprising a sequence set forth in SEQ ID NO: 4;
(viii) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker and a CR2 LDLR domain comprising a sequence set forth in SEQ ID NO: 2;
(ix) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker and a CR2 LDLR domain comprising a sequence set forth in SEQ ID NO: 2;
(x) the immunoglobulin Fc comprising a sequence set forth in SEQ ID NO: 4, a GS linker, a CR3 LDLR domain comprising a sequence set forth in SEQ ID NO: 3, a GSGGS linker, a CR3 LDLR domain comprising a sequence set forth in SEQ ID NO: 3, a GGSSG linker, and a CR3 LDLR domain comprising a sequence set forth in SEQ ID NO: 3;
(xi) a CR2 LDLR domain comprising a sequence set forth in SEQ ID NO: 2, a GS linker, the immunoglobulin Fc comprising a sequence set forth in SEQ ID NO: 4, a GS linker, and a CR2 LDLR domain comprising a sequence set forth in SEQ ID NO: 2; or
(xii) a CR3 LDLR domain comprising a sequence set forth in SEQ ID NO: 3, a GS linker, the immunoglobulin Fc comprising a sequence set forth in SEQ ID NO: 4, a GS linker, and a CR3 LDLR domain comprising a sequence set forth in SEQ ID NO: 3.

In one example, the envelope-binding protein of the disclosure comprises, in order from N- to C-terminus:
(i) a CR2 LDLR domain comprising a sequence set forth in SEQ ID NO: 2, a GS linker and the immunoglobulin Fc comprising a sequence set forth in SEQ ID NO: 4;
(ii) a CR2 LDLR domain comprising a sequence set forth in SEQ ID NO: 2, a CR3 LDLR domain comprising a sequence set forth in SEQ ID NO: 3, a GS linker and the immunoglobulin Fc comprising a sequence set forth in SEQ ID NO: 4;
(iii) the immunoglobulin Fc comprising a sequence set forth in SEQ ID NO: 4, a GS linker and a CR2 LDLR domain comprising a sequence set forth in SEQ ID NO: 2;
(iv) the immunoglobulin Fc comprising a sequence set forth in SEQ ID NO: 4, a GS linker, a CR2 LDLR domain comprising a sequence set forth in SEQ ID NO: 2 and a CR3 LDLR domain comprising a sequence set forth in SEQ ID NO: 3;
(v) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GS linker and the immunoglobulin Fc comprising a sequence set forth in SEQ ID NO: 4;
(vi) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker, a CR2 LDLR domain comprising a sequence set forth in SEQ ID NO: 2 and the immunoglobulin Fc comprising a sequence set forth in SEQ ID NO: 4;
(vii) a CR3 LDLR domain comprising a sequence set forth in SEQ ID NO: 3, a GSGGS linker, a CR3 LDLR domain comprising a sequence set forth in SEQ ID NO: 3, a GGSSG linker, a CR3 LDLR domain comprising a sequence set forth in SEQ ID NO: 3, a GS linker and the immunoglobulin Fc comprising a sequence set forth in SEQ ID NO: 4;
(viii) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker and a CR2 LDLR domain comprising a sequence set forth in SEQ ID NO: 2;
(ix) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker and a CR2 LDLR domain comprising a sequence set forth in SEQ ID NO: 2;
(x) the immunoglobulin Fc comprising a sequence set forth in SEQ ID NO: 4, a GS linker, a CR3 LDLR domain comprising a sequence set forth in SEQ ID NO: 3, a GSGGS linker, a CR3 LDLR domain comprising a sequence set forth in SEQ ID NO: 3, a GGSSG linker, and a CR3 LDLR domain comprising a sequence set forth in SEQ ID NO: 3;
(xi) a CR2 LDLR domain comprising a sequence set forth in SEQ ID NO: 2, a GS linker, the immunoglobulin Fc comprising a sequence set forth in SEQ ID NO: 4, a GS linker, and a CR2 LDLR domain comprising a sequence set forth in SEQ ID NO: 2; or
(xii) a CR3 LDLR domain comprising a sequence set forth in SEQ ID NO: 3, a GS linker, the immunoglobulin Fc comprising a sequence set forth in SEQ ID NO: 4, a GS linker, and a CR3 LDLR domain comprising a sequence set forth in SEQ ID NO: 3.

In one example, the envelope-binding protein of the disclosure consists of, in order from N- to C-terminus:
(i) a CR2 LDLR domain comprising a sequence set forth in SEQ ID NO: 2, a GS linker and the immunoglobulin Fc comprising a sequence set forth in SEQ ID NO: 4;
(ii) a CR2 LDLR domain comprising a sequence set forth in SEQ ID NO: 2, a CR3 LDLR domain comprising a sequence set forth in SEQ ID NO: 3, a GS linker and the immunoglobulin Fc comprising a sequence set forth in SEQ ID NO: 4;
(iii) the immunoglobulin Fc comprising a sequence set forth in SEQ ID NO: 4, a GS linker and a CR2 LDLR domain comprising a sequence set forth in SEQ ID NO: 2;
(iv) the immunoglobulin Fc comprising a sequence set forth in SEQ ID NO: 4, a GS linker, a CR2 LDLR domain comprising a sequence set forth in SEQ ID NO: 2 and a CR3 LDLR domain comprising a sequence set forth in SEQ ID NO: 3;
(v) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GS linker and the immunoglobulin Fc comprising a sequence set forth in SEQ ID NO: 4;
(vi) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker, a CR2 LDLR domain comprising a sequence set forth in SEQ ID NO: 2 and the immunoglobulin Fc comprising a sequence set forth in SEQ ID NO: 4;
(vii) a CR3 LDLR domain comprising a sequence set forth in SEQ ID NO: 3, a GSGGS linker, a CR3 LDLR domain comprising a sequence set forth in SEQ ID NO: 3, a GGSSG linker, a CR3 LDLR domain comprising a sequence set forth in SEQ ID NO: 3, a GS linker and the immunoglobulin Fc comprising a sequence set forth in SEQ ID NO: 4;
(viii) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker and a CR2 LDLR domain comprising a sequence set forth in SEQ ID NO: 2;
(ix) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker and a CR2 LDLR domain comprising a sequence set forth in SEQ ID NO: 2;
(x) the immunoglobulin Fc comprising a sequence set forth in SEQ ID NO: 4, a GS linker, a CR3 LDLR domain comprising a sequence set forth in SEQ ID NO: 3, a GSGGS linker, a CR3 LDLR domain comprising a sequence set forth in SEQ ID NO: 3, a GGSSG linker, and a CR3 LDLR domain comprising a sequence set forth in SEQ ID NO: 3;
(xi) a CR2 LDLR domain comprising a sequence set forth in SEQ ID NO: 2, a GS linker, the immunoglobulin Fc comprising a sequence set forth in SEQ ID NO: 4, a GS linker, and a CR2 LDLR domain comprising a sequence set forth in SEQ ID NO: 2; or
(xii) a CR3 LDLR domain comprising a sequence set forth in SEQ ID NO: 3, a GS linker, the immunoglobulin Fc comprising a sequence set forth in SEQ ID NO: 4, a GS linker, and a CR3 LDLR domain comprising a sequence set forth in SEQ ID NO: 3.

In one example, the envelope-binding protein of the disclosure comprises, in order from N- to C-terminus a CR2 LDLR domain comprising a sequence set forth in SEQ ID NO: 2, a GS linker and the immunoglobulin Fc comprising a sequence set forth in SEQ ID NO: 4.

In one example, the envelope-binding protein of the disclosure comprises, in order from N- to C-terminus a CR2 LDLR domain comprising a sequence set forth in SEQ ID NO: 2, a CR3 LDLR domain comprising a sequence set forth in SEQ ID NO: 3, a GS linker and the immunoglobulin Fc comprising a sequence set forth in SEQ ID NO: 4.

In one example, the envelope-binding protein of the disclosure comprises, in order from N- to C-terminus the immunoglobulin Fc comprising a sequence set forth in SEQ ID NO: 4, a GS linker and a CR2 LDLR domain comprising a sequence set forth in SEQ ID NO: 2.

In one example, the envelope-binding protein of the disclosure comprises, in order from N- to C-terminus the immunoglobulin Fc comprising a sequence set forth in SEQ ID NO: 4, a GS linker, a CR2 LDLR domain comprising a sequence set forth in SEQ ID NO: 2 and a CR3 LDLR domain comprising a sequence set forth in SEQ ID NO: 3.

In one example, the envelope-binding protein of the disclosure comprises, in order from N- to C-terminus a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GS linker and the immunoglobulin Fc comprising a sequence set forth in SEQ ID NO: 4.

In one example, the envelope-binding protein of the disclosure comprises, in order from N- to C-terminus a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker, a CR2 LDLR domain comprising a sequence set forth in SEQ ID NO: 2 and the immunoglobulin Fc comprising a sequence set forth in SEQ ID NO: 4.

In one example, the envelope-binding protein of the disclosure comprises, in order from N- to C-terminus a CR3 LDLR domain comprising a sequence set forth in SEQ ID NO: 3, a GSGGS linker, a CR3 LDLR domain comprising a sequence set forth in SEQ ID NO: 3, a GGSSG linker, a CR3 LDLR domain comprising a sequence set forth in SEQ ID NO: 3, a GS linker and the immunoglobulin Fc comprising a sequence set forth in SEQ ID NO: 4.

In one example, the envelope-binding protein of the disclosure comprises, in order from N- to C-terminus the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker and a CR2 LDLR domain comprising a sequence set forth in SEQ ID NO: 2.

In one example, the envelope-binding protein of the disclosure comprises, in order from N- to C-terminus the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker and a CR2 LDLR domain comprising a sequence set forth in SEQ ID NO: 2.

In one example, the envelope-binding protein of the disclosure comprises, in order from N- to C-terminus the immunoglobulin Fc comprising a sequence set forth in SEQ ID NO: 4, a GS linker, a CR3 LDLR domain comprising a sequence set forth in SEQ ID NO: 3, a GSGGS linker, a CR3 LDLR domain comprising a sequence set forth in SEQ ID NO: 3, a GGSSG linker, and a CR3 LDLR domain comprising a sequence set forth in SEQ ID NO: 3.

In one example, the envelope-binding protein of the disclosure comprises, in order from N- to C-terminus a CR2 LDLR domain comprising a sequence set forth in SEQ ID NO: 2, a GS linker, the immunoglobulin Fc comprising a sequence set forth in SEQ ID NO: 4, a GS linker, and a CR2 LDLR domain comprising a sequence set forth in SEQ ID NO: 2.

In one example, the envelope-binding protein of the disclosure comprises, in order from N- to C-terminus a CR3 LDLR domain comprising a sequence set forth in SEQ ID NO: 3, a GS linker, the immunoglobulin Fc comprising a sequence set forth in SEQ ID NO: 4, a GS linker, and a CR3 LDLR domain comprising a sequence set forth in SEQ ID NO: 3.

In one example, the envelope-binding protein of the disclosure comprises, or consists of, in order from N- to C-terminus:
(i) a CR2 LDLR domain comprising a sequence set forth in SEQ ID NO: 32, a GS linker and the immunoglobulin Fc comprising a sequence set forth in SEQ ID NO: 4;
(ii) a CR2 LDLR domain comprising a sequence set forth in SEQ ID NO: 32, a CR3 LDLR domain comprising a sequence set forth in SEQ ID NO: 33, a GS linker and the immunoglobulin Fc comprising a sequence set forth in SEQ ID NO: 4;
(iii) the immunoglobulin Fc comprising a sequence set forth in SEQ ID NO: 4, a GS linker and a CR2 LDLR domain comprising a sequence set forth in SEQ ID NO: 32;
(iv) the immunoglobulin Fc comprising a sequence set forth in SEQ ID NO: 4, a GS linker, a CR2 LDLR domain comprising a sequence set forth in SEQ ID NO: 32 and a CR3 LDLR domain comprising a sequence set forth in SEQ ID NO: 33;
(v) a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GS linker and the immunoglobulin Fc comprising a sequence set forth in SEQ ID NO: 4;
(vi) a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GGGGS linker, a CR2 LDLR domain comprising a sequence set forth in SEQ ID NO: 32, a GSGSG linker, a CR2 LDLR domain comprising a sequence set forth in SEQ ID NO: 32 and the immunoglobulin Fc comprising a sequence set forth in SEQ ID NO: 4;
(vii) a CR3 LDLR domain comprising a sequence set forth in SEQ ID NO: 33, a GSGGS linker, a CR3 LDLR domain comprising a sequence set forth in SEQ ID NO: 33, a GGSSG linker, a CR3 LDLR domain comprising a sequence set forth in SEQ ID NO: 33, a GS linker and the immunoglobulin Fc comprising a sequence set forth in SEQ ID NO: 4;
(viii) the immunoglobulin Fc comprising a sequence set forth in SEQ ID NO: 4, a GS linker, a CR2 LDLR domain comprising a sequence set forth in SEQ ID NO: 32, a GSGGS linker, a CR2 LDLR domain comprising a sequence set forth in SEQ ID NO: 32, a GGSSG linker and a CR2 LDLR domain comprising a sequence set forth in SEQ ID NO: 32;
(ix) the immunoglobulin Fc comprising a sequence set forth in SEQ ID NO: 4, a GS linker, a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GGGGS linker, a CR2 LDLR domain comprising a sequence set forth in SEQ ID NO: 32, a GSGSG linker and a CR2 LDLR domain comprising a sequence set forth in SEQ ID NO: 32;
(x) the immunoglobulin Fc comprising a sequence set forth in SEQ ID NO: 4, a GS linker, a CR3 LDLR domain comprising a sequence set forth in SEQ ID NO: 33, a GSGGS linker, a CR3 LDLR domain comprising a sequence set forth in SEQ ID NO: 33, a GGSSG linker, and a CR3 LDLR domain comprising a sequence set forth in SEQ ID NO: 33;
(xi) a CR2 LDLR domain comprising a sequence set forth in SEQ ID NO: 32, a GS linker, the immunoglobulin Fc comprising a sequence set forth in SEQ ID NO: 4, a GS linker, and a CR2 LDLR domain comprising a sequence set forth in SEQ ID NO: 32; or
(xii) a CR3 LDLR domain comprising a sequence set forth in SEQ ID NO: 33, a GS linker, the immunoglobulin Fc comprising a sequence set forth in SEQ ID NO: 4, a GS linker, and a CR3 LDLR domain comprising a sequence set forth in SEQ ID NO: 33.

In one example, the envelope-binding protein of the disclosure comprises, or consists of, in order from N- to C-terminus a CR2 LDLR domain comprising a sequence set forth in SEQ ID NO: 32, a GS linker and the immunoglobulin Fc comprising a sequence set forth in SEQ ID NO: 4.

In one example, the envelope-binding protein of the disclosure comprises, or consists of, in order from N- to C-terminus a CR2 LDLR domain comprising a sequence set forth in SEQ ID NO: 32, a CR3 LDLR domain comprising a sequence set forth in SEQ ID NO: 33, a GS linker and the immunoglobulin Fc comprising a sequence set forth in SEQ ID NO: 4.

In one example, the envelope-binding protein of the disclosure comprises, or consists of, in order from N- to C-terminus the immunoglobulin Fc comprising a sequence set forth in SEQ ID NO: 4, a GS linker and a CR2 LDLR domain comprising a sequence set forth in SEQ ID NO: 32.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising a sequence set forth in SEQ ID NO: 32 and a CR3 LDLR domain comprising a sequence set forth in SEQ ID NO: 33.

In one example, the envelope-binding protein of the disclosure comprises, or consists of, in order from N- to C-terminus a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GS linker and the immunoglobulin Fc comprising a sequence set forth in SEQ ID NO: 4.

In one example, the envelope-binding protein of the disclosure comprises, or consists of, in order from N- to C-terminus a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GGGGS linker, a CR2 LDLR domain comprising a sequence set forth in SEQ ID NO: 32, a GSGSG linker, a CR2 LDLR domain comprising a sequence set forth in SEQ ID NO: 32 and the immunoglobulin Fc comprising a sequence set forth in SEQ ID NO: 4.

In one example, the envelope-binding protein of the disclosure comprises, or consists of, in order from N- to C-terminus a CR3 LDLR domain comprising a sequence set forth in SEQ ID NO: 33, a GSGGS linker, a CR3 LDLR domain comprising a sequence set forth in SEQ ID NO: 33, a GGSSG linker, a CR3 LDLR domain comprising a sequence set forth in SEQ ID NO: 33, a GS linker and the immunoglobulin Fc comprising a sequence set forth in SEQ ID NO: 4.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising a sequence set forth in SEQ ID NO: 32, a GSGGS linker, a CR2 LDLR domain comprising a sequence set forth in SEQ ID NO: 32, a GGSSG linker and a CR2 LDLR domain comprising a sequence set forth in SEQ ID NO: 32.

In one example, the envelope-binding protein of the disclosure comprises, or consists of, in order from N- to C-terminus the immunoglobulin Fc comprising a sequence set forth in SEQ ID NO: 4, a GS linker, a CR2 LDLR domain comprising a sequence set forth in SEQ ID NO: 32, a GSGGS linker, a CR2 LDLR domain comprising a sequence set forth in SEQ ID NO: 32, a GGSSG linker, a CR2 LDLR domain comprising a sequence set forth in SEQ ID NO: 32, a GGGGS linker, a CR2 LDLR domain comprising a sequence set forth in SEQ ID NO: 32, a GSGSG linker and a CR2 LDLR domain comprising a sequence set forth in SEQ ID NO: 32.

In one example, the envelope-binding protein of the disclosure comprises, or consists of, in order from N- to C-terminus the immunoglobulin Fc comprising a sequence set forth in SEQ ID NO: 4, a GS linker, a CR3 LDLR domain comprising a sequence set forth in SEQ ID NO: 33, a GSGGS linker, a CR3 LDLR domain comprising a sequence set forth in SEQ ID NO: 33, a GGSSG linker, and a CR3 LDLR domain comprising a sequence set forth in SEQ ID NO: 33.

In one example, the envelope-binding protein of the disclosure comprises, or consists of, in order from N- to C-terminus a CR2 LDLR domain comprising a sequence set forth in SEQ ID NO: 32, a GS linker, the immunoglobulin Fc comprising a sequence set forth in SEQ ID NO: 4, a GS linker, and a CR2 LDLR domain comprising a sequence set forth in SEQ ID NO: 32.

In one example, the envelope-binding protein of the disclosure comprises, or consists of, in order from N- to C-terminus a CR3 LDLR domain comprising a sequence set forth in SEQ ID NO: 33, a GS linker, the immunoglobulin Fc comprising a sequence set forth in SEQ ID NO: 4, a GS linker, and a CR3 LDLR domain comprising a sequence set forth in SEQ ID NO: 33.

In one example, the protein comprises or consists of a sequence set forth in any one of SEQ ID NOs: 7 to 18. In one example, the protein comprises or consists of a sequence set forth in any one of SEQ ID NOs: 7 to 18, optionally wherein the protein lacks the signal peptide corresponding to amino acids 1 to 19 of SEQ ID NOs: 7 to 18.

In one example, the protein sequence does not comprise a signal sequence (or signal peptide sequence). For example, the protein sequence does not comprise a signal sequence comprising a sequence set forth in SEQ ID NO: 5 or SEQ ID NO: 6.

In one example, the protein comprises or consists of a sequence set forth in SEQ ID NO: 7. For example, the protein comprises a sequence set forth in SEQ ID NO: 7. In another example, the protein consists of a sequence set forth in SEQ ID NO: 7.

In one example, the protein comprises or consists of a sequence set forth in SEQ ID NO: 7, optionally wherein the protein lacks the signal peptide corresponding to amino acids 1 to 19 of SEQ ID NO: 7. For example, the protein comprises a sequence set forth in SEQ ID NO: 7, optionally wherein the protein lacks the signal peptide corresponding to amino acids 1 to 19 of SEQ ID NO: 7. In another example, the protein consists of a sequence set forth in SEQ ID NO: 7, optionally wherein the protein lacks the signal peptide corresponding to amino acids 1 to 19 of SEQ ID NO: 7.

In one example, the protein comprises or consists of a sequence comprising amino acids 20 to 294 of SEQ ID NO: 7. For example, the protein comprises a sequence comprising amino acids 20 to 294 of SEQ ID NO: 7. In another example, the protein consists of a sequence comprising amino acids 20 to 294 of SEQ ID NO: 7.

In one example, the protein comprises or consists of a sequence set forth in SEQ ID NO: 8. For example, the protein comprises a sequence set forth in SEQ ID NO: 8. In another example, the protein consists of a sequence set forth in SEQ ID NO: 8.

In one example, the protein comprises or consists of a sequence set forth in SEQ ID NO: 8, optionally wherein the protein lacks the signal peptide corresponding to amino acids 1 to 19 of SEQ ID NO: 8. For example, the protein comprises a sequence set forth in SEQ ID NO: 8, optionally wherein the protein lacks the signal peptide corresponding to amino acids 1 to 19 of SEQ ID NO: 8. In another example, the protein consists of a sequence set forth in SEQ ID NO: 8, optionally wherein the protein lacks the signal peptide corresponding to amino acids 1 to 19 of SEQ ID NO: 8.

In one example, the protein comprises or consists of a sequence comprising amino acids 20 to 333 of SEQ ID NO: 8. For example, the protein comprises a sequence comprising amino acids 20 to 333 of SEQ ID NO: 8. In another example, the protein consists of a sequence comprising amino acids 20 to 333 of SEQ ID NO: 8.

In one example, the protein comprises or consists of a sequence set forth in SEQ ID NO: 9. For example, the protein comprises a sequence set forth in SEQ ID NO: 9. In another example, the protein consists of a sequence set forth in SEQ ID NO: 9.

In one example, the protein comprises or consists of a sequence set forth in SEQ ID NO: 9, optionally wherein the protein lacks the signal peptide corresponding to amino acids 1 to 19 of SEQ ID NO: 9. For example, the protein comprises a sequence set forth in SEQ ID NO: 9, optionally wherein the protein lacks the signal peptide corresponding to amino acids 1 to 19 of SEQ ID NO: 9. In another example, the protein consists of a sequence set forth in SEQ ID NO: 9, optionally wherein the protein lacks the signal peptide corresponding to amino acids 1 to 19 of SEQ ID NO: 9.

In one example, the protein comprises or consists of a sequence comprising amino acids 20 to 294 of SEQ ID NO: 9. For example, the protein comprises a sequence comprising amino acids 20 to 294 of SEQ ID NO: 9. In another example, the protein consists of a sequence comprising amino acids 20 to 294 of SEQ ID NO: 9.

In one example, the protein comprises or consists of a sequence set forth in SEQ ID NO: 10. For example, the protein comprises a sequence set forth in SEQ ID NO: 10. In another example, the protein consists of a sequence set forth in SEQ ID NO: 10.

In one example, the protein comprises or consists of a sequence set forth in SEQ ID NO: 10, optionally wherein the protein lacks the signal peptide corresponding to amino acids 1 to 19 of SEQ ID NO: 10. For example, the protein comprises a sequence set forth in SEQ ID NO: 10, optionally wherein the protein lacks the signal peptide corresponding to amino acids 1 to 19 of SEQ ID NO: 10. In another example, the protein consists of a sequence set forth in SEQ ID NO: 10, optionally wherein the protein lacks the signal peptide corresponding to amino acids 1 to 19 of SEQ ID NO: 10.

In one example, the protein comprises or consists of a sequence comprising amino acids 20 to 333 of SEQ ID NO: 10. For example, the protein comprises a sequence comprising amino acids 20 to 333 of SEQ ID NO: 10. In another example, the protein consists of a sequence comprising amino acids 20 to 333 of SEQ ID NO: 10.

In one example, the protein comprises or consists of a sequence set forth in SEQ ID NO: 11. For example, the protein comprises a sequence set forth in SEQ ID NO: 11. In another example, the protein consists of a sequence set forth in SEQ ID NO: 11.

In one example, the protein comprises or consists of a sequence set forth in SEQ ID NO: 11, optionally wherein the protein lacks the signal peptide corresponding to amino acids 1 to 19 of SEQ ID NO: 11. For example, the protein comprises a sequence set forth in SEQ ID NO: 11, optionally wherein the protein lacks the signal peptide corresponding to amino acids 1 to 19 of SEQ ID NO: 11. In another example, the protein consists of a sequence set forth in SEQ ID NO: 11, optionally wherein the protein lacks the signal peptide corresponding to amino acids 1 to 19 of SEQ ID NO: 11.

In one example, the protein comprises or consists of a sequence comprising amino acids 20 to 386 of SEQ ID NO: 11. For example, the protein comprises a sequence comprising amino acids 20 to 386 of SEQ ID NO: 11. In another example, the protein consists of a sequence comprising amino acids 20 to 386 of SEQ ID NO: 11.

In one example, the protein comprises or consists of a sequence set forth in SEQ ID NO: 12. For example, the protein comprises a sequence set forth in SEQ ID NO: 12. In another example, the protein consists of a sequence set forth in SEQ ID NO: 12.

In one example, the protein comprises or consists of a sequence set forth in SEQ ID NO: 12, optionally wherein the protein lacks the signal peptide corresponding to amino acids 1 to 19 of SEQ ID NO: 12. For example, the protein comprises a sequence set forth in SEQ ID NO: 12, optionally wherein the protein lacks the signal peptide corresponding to amino acids 1 to 19 of SEQ ID NO: 12. In another example, the protein consists of a sequence set forth in SEQ ID NO: 12, optionally wherein the protein lacks the signal peptide corresponding to amino acids 1 to 19 of SEQ ID NO: 12.

In one example, the protein comprises or consists of a sequence comprising amino acids 20 to 478 of SEQ ID NO: 12. For example, the protein comprises a sequence comprising amino acids 20 to 478 of SEQ ID NO: 12. In another example, the protein consists of a sequence comprising amino acids 20 to 478 of SEQ ID NO: 12.

In one example, the protein comprises or consists of a sequence set forth in SEQ ID NO: 13. For example, the protein comprises of a sequence set forth in SEQ ID NO: 13. In another example, the protein consists of a sequence set forth in SEQ ID NO: 13.

In one example, the protein comprises or consists of a sequence set forth in SEQ ID NO: 13, optionally wherein the protein lacks the signal peptide corresponding to amino acids 1 to 19 of SEQ ID NO: 13. For example, the protein comprises of a sequence set forth in SEQ ID NO: 13, optionally wherein the protein lacks the signal peptide corresponding to amino acids 1 to 19 of SEQ ID NO: 13. In another example, the protein consists of a sequence set forth in SEQ ID NO: 13, optionally wherein the protein lacks the signal peptide corresponding to amino acids 1 to 19 of SEQ ID NO: 13.

In one example, the protein comprises or consists of a sequence comprising amino acids 20 to 380 of SEQ ID NO: 13. For example, the protein comprises a sequence comprising amino acids 20 to 380 of SEQ ID NO: 13. In another example, the protein consists of a sequence comprising amino acids 20 to 380 of SEQ ID NO: 13.

In one example, the protein comprises or consists of a sequence set forth in SEQ ID NO: 14. For example, the protein comprises of a sequence set forth in SEQ ID NO: 14. In another example, the protein consists of a sequence set forth in SEQ ID NO: 14.

In one example, the protein comprises or consists of a sequence set forth in SEQ ID NO: 14, optionally wherein the protein lacks the signal peptide corresponding to amino acids 1 to 19 of SEQ ID NO: 14. For example, the protein comprises of a sequence set forth in SEQ ID NO: 14, optionally wherein the protein lacks the signal peptide corresponding to amino acids 1 to 19 of SEQ ID NO: 14. In another example, the protein consists of a sequence set forth in SEQ ID NO: 14, optionally wherein the protein lacks the signal peptide corresponding to amino acids 1 to 19 of SEQ ID NO: 14.

In one example, the protein comprises or consists of a sequence comprising amino acids 20 to 386 of SEQ ID NO: 14. For example, the protein comprises a sequence comprising amino acids 20 to 386 of SEQ ID NO: 14. In another example, the protein consists of a sequence comprising amino acids 20 to 386 of SEQ ID NO: 14.

In one example, the protein comprises or consists of a sequence set forth in SEQ ID NO: 15. For example, the protein comprises of a sequence set forth in SEQ ID NO: 15. In another example, the protein consists of a sequence set forth in SEQ ID NO: 15.

In one example, the protein comprises or consists of a sequence set forth in SEQ ID NO: 15, optionally wherein the protein lacks the signal peptide corresponding to amino acids 1 to 19 of SEQ ID NO: 15. For example, the protein comprises of a sequence set forth in SEQ ID NO: 15, optionally wherein the protein lacks the signal peptide corresponding to amino acids 1 to 19 of SEQ ID NO: 15. In another example, the protein consists of a sequence set forth in SEQ ID NO: 15, optionally wherein the protein lacks the signal peptide corresponding to amino acids 1 to 19 of SEQ ID NO: 15.

In one example, the protein comprises or consists of a sequence comprising amino acids 20 to 478 of SEQ ID NO: 15. For example, the protein comprises a sequence comprising amino acids 20 to 478 of SEQ ID NO: 15. In another example, the protein consists of a sequence comprising amino acids 20 to 478 of SEQ ID NO: 15.

In one example, the protein comprises or consists of a sequence set forth in SEQ ID NO: 16. For example, the protein comprises of a sequence set forth in SEQ ID NO: 16. In another example, the protein consists of a sequence set forth in SEQ ID NO: 16.

In one example, the protein comprises or consists of a sequence set forth in SEQ ID NO: 16, optionally wherein the protein lacks the signal peptide corresponding to amino acids 1 to 19 of SEQ ID NO: 16. For example, the protein comprises of a sequence set forth in SEQ ID NO: 16, optionally wherein the protein lacks the signal peptide corresponding to amino acids 1 to 19 of SEQ ID NO: 16. In another example, the protein consists of a sequence set forth in SEQ ID NO: 16, optionally wherein the protein lacks the signal peptide corresponding to amino acids 1 to 19 of SEQ ID NO: 16.

In one example, the protein comprises or consists of a sequence comprising amino acids 20 to 380 of SEQ ID NO: 16. For example, the protein comprises a sequence comprising amino acids 20 to 380 of SEQ ID NO: 16. In another example, the protein consists of a sequence comprising amino acids 20 to 380 of SEQ ID NO: 16.

In one example, the protein comprises or consists of a sequence set forth in SEQ ID NO: 17. For example, the protein comprises of a sequence set forth in SEQ ID NO: 17. In another example, the protein consists of a sequence set forth in SEQ ID NO: 17.

In one example, the protein comprises or consists of a sequence set forth in SEQ ID NO: 17, optionally wherein the protein lacks the signal peptide corresponding to amino acids 1 to 19 of SEQ ID NO: 17. For example, the protein comprises of a sequence set forth in SEQ ID NO: 17, optionally wherein the protein lacks the signal peptide corresponding to amino acids 1 to 19 of SEQ ID NO: 17. In another example, the protein consists of a sequence set forth in SEQ ID NO: 17, optionally wherein the protein lacks the signal peptide corresponding to amino acids 1 to 19 of SEQ ID NO: 17.

In one example, the protein comprises or consists of a sequence comprising amino acids 20 to 333 of SEQ ID NO: 17. For example, the protein comprises a sequence comprising amino acids 20 to 333 of SEQ ID NO: 17. In another example, the protein consists of a sequence comprising amino acids 20 to 333 of SEQ ID NO: 17.

In one example, the protein comprises or consists of a sequence set forth in SEQ ID NO: 18. For example, the protein comprises of a sequence set forth in SEQ ID NO: 18. In another example, the protein consists of a sequence set forth in SEQ ID NO: 18.

In one example, the protein comprises or consists of a sequence set forth in SEQ ID NO: 18, optionally wherein the protein lacks the signal peptide corresponding to amino acids 1 to 19 of SEQ ID NO: 18. For example, the protein comprises of a sequence set forth in SEQ ID NO: 18, optionally wherein the protein lacks the signal peptide corresponding to amino acids 1 to 19 of SEQ ID NO: 18. In another example, the protein consists of a sequence set forth in SEQ ID NO: 18, optionally wherein the protein lacks the signal peptide corresponding to amino acids 1 to 19 of SEQ ID NO: 18.

In one example, the protein comprises or consists of a sequence comprising amino acids 20 to 333 of SEQ ID NO: 18. For example, the protein comprises a sequence comprising amino acids 20 to 333 of SEQ ID NO: 18. In another example, the protein consists of a sequence comprising amino acids 20 to 333 of SEQ ID NO: 18.

In one example, the protein comprises or consists of a sequence expressed from or encoded by a nucleic acid comprising a sequence set forth in any one of SEQ ID NOs: 19 to 30.

In one example, the protein comprises or consists of a sequence expressed from or encoded by a nucleic acid comprising a sequence set forth in SEQ ID NO: 19. For example, the protein comprises a sequence expressed from or encoded by a nucleic acid comprising a sequence set forth in SEQ ID NO: 19. In another example, the protein consists of a sequence expressed from or encoded by a nucleic acid comprising a sequence set forth in SEQ ID NO: 19.

In one example, the protein comprises or consists of a sequence expressed from or encoded by a nucleic acid comprising a sequence set forth in SEQ ID NO: 20. For example, the protein comprises a sequence expressed from or encoded by a nucleic acid comprising a sequence set forth in SEQ ID NO: 20. In another example, the protein consists of a sequence expressed from or encoded by a nucleic acid comprising a sequence set forth in SEQ ID NO: 20.

In one example, the protein comprises or consists of a sequence expressed from or encoded by a nucleic acid comprising a sequence set forth in SEQ ID NO: 21. For example, the protein comprises a sequence expressed from or encoded by a nucleic acid comprising a sequence set forth in SEQ ID NO: 21. In another example, the protein consists of a sequence expressed from or encoded by a nucleic acid comprising a sequence set forth in SEQ ID NO: 21.

In one example, the protein comprises or consists of a sequence expressed from or encoded by a nucleic acid comprising a sequence set forth in SEQ ID NO: 21, optionally wherein the protein lacks the intronic sequence corresponding to nucleotides 59 to 140 of SEQ ID NO: 21. For example, the protein comprises a sequence expressed from or encoded by a nucleic acid comprising a sequence set forth in SEQ ID NO: 21, optionally wherein the protein lacks the intronic sequence corresponding to nucleotides 59 to 140 of SEQ ID NO: 21. In another example, the protein consists of a sequence expressed from or encoded by a nucleic acid comprising a sequence set forth in SEQ ID NO: 21, optionally wherein the protein lacks the intronic sequence corresponding to nucleotides 59 to 140 of SEQ ID NO: 21.

In one example, the protein comprises or consists of a sequence expressed from or encoded by a nucleic acid comprising a sequence set forth in SEQ ID NO: 22. For example, the protein comprises a sequence expressed from or encoded by a nucleic acid comprising a sequence set forth in SEQ ID NO: 22. In another example, the protein consists of a sequence expressed from or encoded by a nucleic acid comprising a sequence set forth in SEQ ID NO: 22.

In one example, the protein comprises or consists of a sequence expressed from or encoded by a nucleic acid comprising a sequence set forth in SEQ ID NO: 22, optionally wherein the protein lacks the intronic sequence corresponding to nucleotides 59 to 140 of SEQ ID NO: 22. For example, the protein comprises a sequence expressed from or encoded by a nucleic acid comprising a sequence set forth in SEQ ID NO: 22, optionally wherein the protein lacks the intronic sequence corresponding to nucleotides 59 to 140 of SEQ ID NO: 22. In another example, the protein consists of a sequence expressed from or encoded by a nucleic acid comprising a sequence set forth in SEQ ID NO: 22, optionally wherein the protein lacks the intronic sequence corresponding to nucleotides 59 to 140 of SEQ ID NO: 22.

In one example, the protein comprises or consists of a sequence expressed from or encoded by a nucleic acid comprising a sequence set forth in SEQ ID NO: 23. For example, the protein comprises a sequence expressed from or encoded by a nucleic acid comprising a sequence set forth in SEQ ID NO: 23. In another example, the protein consists of a sequence expressed from or encoded by a nucleic acid comprising a sequence set forth in SEQ ID NO: 23.

In one example, the protein comprises or consists of a sequence expressed from or encoded by a nucleic acid comprising a sequence set forth in SEQ ID NO: 24. For example, the protein comprises a sequence expressed from or encoded by a nucleic acid comprising a sequence set forth in SEQ ID NO: 24. In another example, the protein consists of a sequence expressed from or encoded by a nucleic acid comprising a sequence set forth in SEQ ID NO: 24.

In one example, the protein comprises or consists of a sequence expressed from or encoded by a nucleic acid comprising a sequence set forth in SEQ ID NO: 25. For example, the protein comprises a sequence expressed from or encoded by a nucleic acid comprising a sequence set forth in SEQ ID NO: 25. In another example, the protein consists of a sequence expressed from or encoded by a nucleic acid comprising a sequence set forth in SEQ ID NO: 25.

In one example, the protein comprises or consists of a sequence expressed from or encoded by a nucleic acid comprising a sequence set forth in SEQ ID NO: 26. For example, the protein comprises a sequence expressed from or encoded by a nucleic acid comprising a sequence set forth in SEQ ID NO: 26. In another example, the protein consists of a sequence expressed from or encoded by a nucleic acid comprising a sequence set forth in SEQ ID NO: 26.

In one example, the protein comprises or consists of a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 26. For example, the protein comprises a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 26. In another example, the protein consists of a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 26.

In one example, the protein comprises or consists of a sequence expressed from or encoded by a nucleic acid comprising a sequence set forth in SEQ ID NO: 27. For example, the protein comprises a sequence expressed from or encoded by a nucleic acid comprising a sequence set forth in SEQ ID NO: 27. In another example, the protein consists of a sequence expressed from or encoded by a nucleic acid comprising a sequence set forth in SEQ ID NO: 27.

In one example, the protein comprises or consists of a sequence expressed from or encoded by a nucleic acid comprising a sequence set forth in SEQ ID NO: 27, optionally wherein the protein lacks the intronic sequence corresponding to nucleotides 59 to 140 of SEQ ID NO: 27. For example, the protein comprises a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 27. In another example, the protein consists of a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 27.

In one example, the protein comprises or consists of a sequence expressed from or encoded by a nucleic acid comprising a sequence set forth in SEQ ID NO: 28. For example, the protein comprises a sequence expressed from or encoded by a nucleic acid comprising a sequence set forth in SEQ ID NO: 28. In another example, the protein consists of a sequence expressed from or encoded by a nucleic acid comprising a sequence set forth in SEQ ID NO: 28.

In one example, the protein comprises or consists of a sequence expressed from or encoded by a nucleic acid comprising a sequence set forth in SEQ ID NO: 28, optionally wherein the protein lacks the intronic sequence corresponding to nucleotides 59 to 140 of SEQ ID NO: 28. For example, the protein comprises a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 28. In another example, the protein consists of a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 28.

In one example, the protein comprises or consists of a sequence expressed from or encoded by a nucleic acid comprising a sequence set forth in SEQ ID NO: 29. For example, the protein comprises a sequence expressed from or encoded by a nucleic acid comprising a sequence set forth in SEQ ID NO: 29. In another example, the protein consists of a sequence expressed from or encoded by a nucleic acid comprising a sequence set forth in SEQ ID NO: 29.

In one example, the protein comprises or consists of a sequence expressed from or encoded by a nucleic acid comprising a sequence set forth in SEQ ID NO: 30. For example, the protein comprises a sequence expressed from or encoded by a nucleic acid comprising a sequence set forth in SEQ ID NO: 30. In another example, the protein consists of a sequence expressed from or encoded by a nucleic acid comprising a sequence set forth in SEQ ID NO: 30.

The present disclosure provides an envelope-binding protein comprising a sequence set forth in any one of SEQ ID NOs: 7 to 18.

The present disclosure provides an envelope-binding protein comprising a sequence set forth in SEQ ID NO: 7.

The present disclosure provides an envelope-binding protein comprising a sequence set forth in SEQ ID NO: 8.

The present disclosure provides an envelope-binding protein comprising a sequence set forth in SEQ ID NO: 9.

The present disclosure provides an envelope-binding protein comprising a sequence set forth in SEQ ID NO: 10.

The present disclosure provides an envelope-binding protein comprising a sequence set forth in SEQ ID NO: 11.

The present disclosure provides an envelope-binding protein comprising a sequence set forth in SEQ ID NO: 12.

The present disclosure provides an envelope-binding protein comprising a sequence set forth in SEQ ID NO: 13.

The present disclosure provides an envelope-binding protein comprising a sequence set forth in SEQ ID NO: 14.

The present disclosure provides an envelope-binding protein comprising a sequence set forth in SEQ ID NO: 15.

The present disclosure provides an envelope-binding protein comprising a sequence set forth in SEQ ID NO: 16.

The present disclosure provides an envelope-binding protein comprising a sequence set forth in SEQ ID NO: 17.

The present disclosure provides an envelope-binding protein comprising a sequence set forth in SEQ ID NO: 18.

The present disclosure provides an envelope-binding protein comprising:
(i) a sequence set forth in SEQ ID NO: 7, optionally wherein the protein lacks the signal peptide corresponding to amino acids 1 to 19 of SEQ ID NO: 7;
(ii) a sequence set forth in SEQ ID NO: 8, optionally wherein the protein lacks the signal peptide corresponding to amino acids 1 to 19 of SEQ ID NO: 8;
(iii) a sequence set forth in SEQ ID NO: 9, optionally wherein the protein lacks the signal peptide corresponding to amino acids 1 to 19 of SEQ ID NO: 9;
(iv) a sequence set forth in SEQ ID NO: 10, optionally wherein the protein lacks the signal peptide corresponding to amino acids 1 to 19 of SEQ ID NO: 10;
(v) a sequence set forth in SEQ ID NO: 11, optionally wherein the protein lacks the signal peptide corresponding to amino acids 1 to 19 of SEQ ID NO: 11;
(vi) a sequence set forth in SEQ ID NO: 12, optionally wherein the protein lacks the signal peptide corresponding to amino acids 1 to 19 of SEQ ID NO: 12;
(vii) a sequence set forth in SEQ ID NO: 13, optionally wherein the protein lacks the signal peptide corresponding to amino acids 1 to 19 of SEQ ID NO: 13;
(viii) a sequence set forth in SEQ ID NO: 14, optionally wherein the protein lacks the signal peptide corresponding to amino acids 1 to 19 of SEQ ID NO: 14;
(ix) a sequence set forth in SEQ ID NO: 15, optionally wherein the protein lacks the signal peptide corresponding to amino acids 1 to 19 of SEQ ID NO: 15;
(x) a sequence set forth in SEQ ID NO: 16, optionally wherein the protein lacks the signal peptide corresponding to amino acids 1 to 19 of SEQ ID NO: 16;
(xi) a sequence set forth in SEQ ID NO: 17, optionally wherein the protein lacks the signal peptide corresponding to amino acids 1 to 19 of SEQ ID NO: 17; or
(xii) a sequence set forth in SEQ ID NO: 18, optionally wherein the protein lacks the signal peptide corresponding to amino acids 1 to 19 of SEQ ID NO: 18.

The present disclosure provides an envelope-binding protein comprising:
(i) a sequence comprising amino acids 20 to 294 of SEQ ID NO: 7;
(ii) a sequence comprising amino acids 20 to 333 of SEQ ID NO: 8;
(iii) a sequence comprising amino acids 20 to 294 of SEQ ID NO: 9;
(iv) a sequence comprising amino acids 20 to 333 of SEQ ID NO: 10;
(v) a sequence comprising amino acids 20 to 386 of SEQ ID NO: 11;
(vi) a sequence comprising amino acids 20 to 478 of SEQ ID NO: 12;
(vii) a sequence comprising amino acids 20 to 380 of SEQ ID NO: 13;
(viii) a sequence comprising amino acids 20 to 386 of SEQ ID NO: 14;
(ix) a sequence comprising amino acids 20 to 478 of SEQ ID NO: 15;
(x) a sequence comprising amino acids 20 to 380 of SEQ ID NO: 16;
(xi) a sequence comprising amino acids 20 to 333 of SEQ ID NO: 17; or
(xii) a sequence comprising amino acids 20 to 333 of SEQ ID NO: 18.

The present disclosure provides a nucleic acid encoding or expressing the envelope-binding protein of the present disclosure.

The present disclosure provides an envelope binding protein comprising a sequence expressed from or encoded by a nucleic acid comprising a sequence set forth in any one of SEQ ID NOs: 19 to 30.

The present disclosure provides an envelope binding protein comprising a sequence expressed from or encoded by a nucleic acid comprising a sequence set forth in SEQ ID NO: 19.

The present disclosure provides an envelope binding protein comprising a sequence expressed from or encoded by a nucleic acid comprising a sequence set forth in SEQ ID NO: 20.

The present disclosure provides an envelope binding protein comprising a sequence expressed from or encoded by a nucleic acid comprising a sequence set forth in SEQ ID NO: 21.

The present disclosure provides an envelope-binding protein comprising a sequence expressed from or encoded by a nucleic acid comprising a sequence set forth in SEQ ID NO: 21, optionally wherein the protein lacks the intronic sequence corresponding to nucleotides 59 to 140 of SEQ ID NO: 21.

The present disclosure provides an envelope binding protein comprising a sequence expressed from or encoded by a nucleic acid comprising a sequence set forth in SEQ ID NO: 22.

The present disclosure provides an envelope-binding protein comprising a sequence expressed from or encoded by a nucleic acid comprising a sequence set forth in SEQ ID NO: 22, optionally wherein the protein lacks the intronic sequence corresponding to nucleotides 59 to 140 of SEQ ID NO: 22.

The present disclosure provides an envelope binding protein comprising a sequence expressed from or encoded by a nucleic acid comprising a sequence set forth in SEQ ID NO: 23.

The present disclosure provides an envelope binding protein comprising a sequence expressed from or encoded by a nucleic acid comprising a sequence set forth in SEQ ID NO: 24.

The present disclosure provides an envelope binding protein comprising a sequence expressed from or encoded by a nucleic acid comprising a sequence set forth in SEQ ID NO: 25.

The present disclosure provides an envelope binding protein comprising a sequence expressed from or encoded by a nucleic acid comprising a sequence set forth in SEQ ID NO: 26.

The present disclosure provides an envelope-binding protein comprising a sequence expressed from or encoded by a nucleic acid comprising a sequence set forth in SEQ ID NO: 26, optionally wherein the protein lacks the intronic sequence corresponding to nucleotides 59 to 140 of SEQ ID NO: 26.

The present disclosure provides an envelope binding protein comprising a sequence expressed from or encoded by a nucleic acid comprising a sequence set forth in SEQ ID NO: 27.

The present disclosure provides an envelope-binding protein comprising a sequence expressed from or encoded by a nucleic acid comprising a sequence set forth in SEQ ID NO: 27, optionally wherein the protein lacks the intronic sequence corresponding to nucleotides 59 to 140 of SEQ ID NO: 27.

The present disclosure provides an envelope binding protein comprising a sequence expressed from or encoded by a nucleic acid comprising a sequence set forth in SEQ ID NO: 28.

The present disclosure provides an envelope-binding protein comprising a sequence expressed from or encoded by a nucleic acid comprising a sequence set forth in SEQ ID NO: 28, optionally wherein the protein lacks the intronic sequence corresponding to nucleotides 59 to 140 of SEQ ID NO: 28.

The present disclosure provides an envelope binding protein comprising a sequence expressed from or encoded by a nucleic acid comprising a sequence set forth in SEQ ID NO: 29.

The present disclosure provides an envelope binding protein comprising a sequence expressed from or encoded by a nucleic acid comprising a sequence set forth in SEQ ID NO: 30.

In one example, the disclosure provides an envelope-binding protein for use in production of an enveloped virus in a cell culture, wherein the cell culture comprises culturing a cell line in a cell culture medium.

In one example, the disclosure provides an envelope-binding protein for use in purification of an enveloped virus from a cell culture.

The present disclosure provides a method of producing an enveloped virus in a cell culture, the method comprising culturing a cell line in a cell culture medium comprising a protein that specifically binds vesicular stomatitis virus G (VSV-G) pseudotyped virus.

In one example, the protein that specifically binds vesicular stomatitis virus G (VSV-G) pseudotyped virus comprises one or more low-density lipoprotein receptor (LDLR) domains. In another example, the protein that specifically binds VSV-G pseudotyped virus is an envelope-binding protein of the disclosure.

In one example, the cell line is a stable producer cell line, i.e., cells having stably incorporated therein the genetic material required to produce the lentivirus. Such cells are distinguished from cells having the genetic elements transiently incorporated therein.

In one example, the cell line expresses the protein that specifically binds vesicular stomatitis virus G (VSV-G) pseudotyped virus. For example, the cells express the protein in addition to the lentivirus.

In one example, the protein that specifically binds vesicular stomatitis virus G (VSV-G) pseudotyped virus is added to the cell culture medium. For example, the protein is added as an excipient to the cell culture medium.

In one example, the protein that specifically binds vesicular stomatitis virus G (VSV-G) pseudotyped virus is added in a sufficient amount to bind VSV-G to competitively inhibit interaction with LDLR expressed on producer cells. For example, the protein is added to the cell culture medium at a concentration of at least 0.001 μg/mL. In one example, the sufficient amount of the protein in the cell culture medium is at least 0.001 μg/mL of cell culture medium. In one example, the sufficient amount of protein in the cell culture medium is between about 0.001 μg/mL and about 100,000 μg/mL of cell culture medium. In one example, the sufficient amount of protein in the cell culture medium is between about 0.01 μg/mL and about 100,000 μg/mL of cell culture medium. In one example, the sufficient amount of protein in the cell culture medium is between about 0.01 μg/mL and about 10,000 μg/mL of cell culture medium. In one example, the sufficient amount of protein in the cell culture medium is between about 0.1 ng/mL and about 100,000 μg/mL of cell culture medium. In one example, the sufficient amount of protein in the cell culture medium is between about 0.1 μg/mL and about 10,000 μg/mL of cell culture medium. In one example, the sufficient amount of protein in the cell culture medium is between about 0.1 μg/mL and about 1,000 μg/mL of cell culture medium. In one example, the sufficient amount of protein in the cell culture medium is between about 0.1 μg/mL and about 100 μg/mL of cell culture medium. In one example, the sufficient amount of protein in the cell culture medium is between about 0.1 μg/mL and about 10 μg/mL of cell culture medium. For example, the sufficient amount of protein in the cell culture medium is about 0.1 μg/mL, or about 0.2 μg/mL, or about 0.3 μg/mL, or about 0.4 μg/mL, or about 0.5 μg/mL, or about 0.6 μg/mL, or about 0.7 μg/mL, or about 0.8 μg/mL, or about 0.9 μg/mL, or about 1 μg/mL of cell culture medium. In another example, the sufficient amount of protein in the cell culture medium is about 1 μg/mL, or about 2 μg/mL, or about 3 μg/mL, or about 4 μg/mL, or about 5 μg/mL, or about 6 μg/mL, or about 7 μg/mL, or about 8 μg/mL, or about 9 μg/mL, or about 10 μg/mL of cell culture medium. In one example, the sufficient amount of protein in the cell culture medium is at least 10 μg/mL. In one example, the sufficient amount of protein in the cell culture medium is at least 100 μg/mL. In one example, the sufficient amount of protein in the cell culture medium is between about 10 μg/mL and about 100 μg/mL of cell culture medium. For example, the sufficient amount of protein in the cell culture medium is 10 μg/mL, or 20 μg/mL, or 30 μg/mL, or 40 μg/mL, or 50 μg/mL, or 60 μg/mL, or 70 μg/mL, or 80 μg/mL, or 90 μg/mL, or 100 μg/mL of cell culture medium. For example, the sufficient amount of protein in the cell culture medium is 0.001 μg/mL of cell culture medium. In one example, the sufficient amount of protein in the cell culture medium is 0.01 μg/mL of cell culture medium. In one example, the sufficient amount of protein in the cell culture medium is 0.1 μg/mL of cell culture medium. In one example, the sufficient amount of protein in the cell culture medium is 1 μg/mL of cell culture medium. In one example, the sufficient amount of protein in the cell culture medium is 10 μg/mL of cell culture medium. In one example, the sufficient amount of protein in the cell culture medium is 100 μg/mL of cell culture medium.

In one example, the cell culture is operated in a batch, fed batch, continuous, semi-continuous, or perfusion mode. In one example, the cell culture is operated in batch mode. In another example, the cell culture is operated in fed batch mode. In a further example, the cell culture is operated in semi-continuous mode. In another example, the cell culture is operated in perfusion mode. In one example, the cell culture is operated in batch and perfusion mode. For example, the cell culture is initially operated in batch mode and subsequently operated in perfusion mode.

In one example, the method comprises dissociating the protein from the VSV-G pseudotyped virus such that transduction of the producer cells with the enveloped virus is induced. For example, the method comprises reducing binding of the protein to VSV-G. In one example, the binding of protein to VSV-G is reduced by at least 50%, or at least 60%, or at least 70%, or at least 80%, or at least 85%, or at least 90%, or at least 95%. In one example, the binding of protein to VSV-G is reduced by at least 50%. In one example, the binding of protein to VSV-G is reduced by at least 60%. In one example, the binding of protein to VSV-G is reduced by at least 70%. In one example, the binding of protein to VSV-G is reduced by at least 80%. In one example, the binding of protein to VSV-G is reduced by at least 85%. In one example, the binding of protein to VSV-G is reduced by at least 90%. In one example, the binding of protein to VSV-G is reduced by at least 95%.

In one example, the binding of the protein to VSV-G is reduced by decreasing the concentration of calcium in the cell culture medium. For example, the concentration of calcium in the cell culture medium is decreased to a concentration of 0.01 mM or less.

In one example, the binding of the protein to VSV-G is reduced by reducing the pH of the cell culture medium to a pH of 6.0 or less.

In one example, the method increases the virus infectious titer yield by at least 2% or 3% or 4% or 5% or 10% or 15% or 20%. In one example, the method increases the virus infectious titer yield by at least 10%.

In one example, the method increases the virus infectious titer yield by at least 1-fold, or 2-fold, or 3-fold, or 4-fold, or 5-fold, or 10-fold, or 15-fold, or 20-fold. In one example, the method increases the virus infectious titer yield by at least 1-fold.

In one example, the cell culture has a volume of greater than about 1 L, about 2 L, about 5 L, about 10 L, about 50 L, about 100 L, about 500 L, about 1000 L, about 5,000 L, about 10,000 L, or about 15,000 L. For example, the cell culture has a volume of between about 1 L and 1000 L. In one example, the cell culture has a volume of about 1 L. In another example, the cell culture has a volume of about 5 L. In a further example, the cell culture has a volume of about 10 L. In one example, the cell culture has a volume of about 50 L. In another example, the cell culture has a volume of about 100 L. In a further example, the cell culture has a volume of about 500 L. In one example, the cell culture has a volume of about 1000 L. In another example, the cell culture has a volume of about 5000 L. In another example, the cell culture has a volume of about 10,000 L. In another example, the cell culture has a volume of about 15,000 L.

In one example, the cell culture is operated at a pH of between 6.0 and 8.0. In one example, the cell culture is at a pH of between about 6.5 and 7.5. For example, the pH is between about 6.90 and about 7.3. In one example, the pH is about 7.1. In one example, the pH is about 6.5. In one example, the pH is about 6.6. In one example, the pH is about 6.7. In one example, the pH is about 6.8. In one example, the pH is about 6.9. In one example, the pH is about 7.0. In one example, the pH is about 7.1. In one example, the pH is about 7.2. In one example, the pH is about 7.3. In one example, the pH is about 7.4. In one example, the pH is about 7.5.

In one example, the cell culture is operated at a temperature of between about 35° C. and 39° C. For example, the cell culture is at a temperature of about 35° C. or about 35.5° C., or about 36° C., or about 36.5° C., or about 37° C., or about 37.5° C., or about 38° C., or about 38.5° C., or about 39° C. In one example, the cell culture is at a temperature of between about 36.5° C. and about 37.5° C. For example, the cell culture is at a temperature of about 37.0° C. In one example, the cell culture is at a temperature of between about 38° C. and about 39° C. For example, the cell culture is at a temperature of about 38.5° C.

In one example, the cell culture is at a pH of between 6.0 and 8.0 and/or at a temperature of between 35-39° C. In one example, the cell culture is at a pH of between 6.0 and 8.0 and/or at a temperature of between 37-38.5° C. For example, the cell culture is at a pH of between about 6.8 and about 7.1 and/or at a temperature of between about 37° C. and about 38.5° C. In one example, the cell culture is at a pH of between about 6.8 and about 7.1 and at a temperature of between about 37° C. and about 38.5° C. In one example, the cell culture is at a pH of about 6.9 to about 7.0 and at a temperature of about 37.0° C.

In one example, the method further comprises purifying the enveloped virus from the cell culture. In one example, the method comprises purifying the enveloped virus from the cell culture, wherein the enveloped virus is bound to the envelope-binding protein.

The present disclosure also provides a method of purifying an enveloped virus from a cell culture, the method comprising (i) culturing a cell line in a cell culture medium comprising the protein that specifically binds vesicular stomatitis virus G (VSV-G) pseudotyped virus and (ii) isolating the enveloped virus from the cell culture, wherein the protein is bound to the enveloped virus.

The present disclosure also provides a method of purifying an enveloped virus from a cell culture, the method comprising (i) culturing a cell line in a cell culture medium comprising the envelope-binding protein of the disclosure and (ii) isolating the enveloped virus from the cell culture, wherein the envelope-binding protein is bound to the enveloped virus.

In one example, the enveloped virus bound to the protein is loaded onto an affinity capture chromatography column in a loading buffer comprising calcium. For example, the affinity capture chromatography column is a protein A chromatography column.

The present disclosure provides a method of purifying an enveloped virus from a cell culture, the method comprising (i) culturing a cell line in a cell culture medium comprising an envelope-binding protein of the disclosure, (ii) harvesting the cell culture medium comprising the enveloped virus, (iii) loading the harvested cell culture medium comprising the enveloped virus onto an affinity chromatography resin and (iv) collecting the enveloped virus.

The present disclosure provides a method of purifying an enveloped virus from a cell culture, the method comprising (i) culturing a cell line in a cell culture medium comprising a protein that specifically binds vesicular stomatitis virus G (VSV-G) pseudotyped virus, (ii) harvesting the cell culture medium comprising the enveloped virus, (iii) loading the harvested cell culture medium comprising the enveloped virus onto an affinity chromatography resin and (iv) collecting the enveloped virus.

The present disclosure also provides a method of purifying an enveloped virus from a cell culture, the method comprising (i) culturing a cell line in a cell culture medium, (ii) harvesting the cell culture medium comprising the enveloped virus, (iii) loading the harvested cell culture medium comprising the enveloped virus onto an affinity chromatography resin comprising an envelope-binding protein of the disclosure and (iv) collecting the enveloped virus.

In one example, the affinity chromatography resin comprises an envelope-binding protein of the disclosure immobilized to a matrix of the affinity chromatography resin.

In one example, the present disclosure provides a method of affinity chromatography, the method comprising binding the enveloped virus to an affinity chromatography resin comprising an envelope-binding protein of the disclosure and collecting the enveloped virus.

In one example, the method comprises (i) loading the harvested cell culture fluid comprising the enveloped virus onto an affinity chromatography resin comprising an envelope-binding protein of the disclosure and (ii) collecting the enveloped virus.

In one example, the harvested cell culture medium is filtered following production of the enveloped virus.

In one example, the concentration of calcium in the affinity capture chromatography loading buffer is at least 0.01 mM.

In one example, the affinity capture chromatography column is eluted with a buffer comprising less than 0.01 mM calcium.

In one example, the enveloped virus is purified using one or more additional purification steps. Methods of purifying the enveloped virus will be apparent to the skilled person and/or are described herein. In one example, purifying the enveloped virus comprises one or more steps selected from the group consisting of clarification filtration, anion exchange chromatography, concentration and diafiltration.

In one example, a method of the disclosure additionally comprises performing sterile filtration. For example, the sterile filtration is performed prior to concentrating and diafiltering the eluted virus. In an alternative example, the sterile filtration is performed after concentrating and diafiltering the eluted virus.

In one example, the method additionally comprises formulating the purified enveloped virus into a pharmaceutical formulation or into a solution suitable for infecting a cell.

The present disclosure also provides a purified enveloped virus produced by the method described herein.

An exemplary enveloped virus is a retrovirus. For example, the retrovirus is a lentivirus. For example, the lentivirus is HIV or a derivative thereof.

In one example, the enveloped virus is a VSV-G pseudotyped virus.

The present disclosure additionally provides a chromatography resin comprising an envelope-binding protein of the disclosure immobilized thereon.

### BRIEF DESCRIPTION OF THE DRAWINGS

FIG. 1 is a schematic illustration showing exemplary envelope-binding protein constructs of the disclosure.

FIG. 2 is a graphical representation showing the binding of VSV-G to recombinant envelope binding proteins as determined by surface plasmon resonance (SPR) in the (A) presence of calcium at pH 7.4, (B) absence of calcium at pH 7.4 and (C) presence of calcium at pH 6.0.

FIG. 3 is a graphical representation showing (A) sensorgrams and (B) a steady state 1:1 fit of VSV-G binding C2C3 Fc.

FIG. 4 is a graphical representation showing (A) sensorgrams and (B) a steady state 1:1 fit of VSV-G binding C2 Fc.

FIG. 5 is a graphical representation showing (A) sensorgrams and (B) a steady state 1:1 fit of VSV-G binding Fc C2C3.

FIG. 6 is a graphical representation showing (A) sensorgrams and (B) a steady state 1:1 fit of VSV-G binding Fc C2.

FIG. 7 is a graphical representation showing specific binding of the envelope-binding protein C2-Fc to VSV-G expressing GPRG cells (iGPRG cells) and not VSV-G negative HEK293T cells.

FIG. 8 is a graphical representation showing binding dissociation of the envelope-binding protein C2-Fc to VSV-G after treatment with Ca2+-free DPBS, 10 mM EDTA DPBS pH7, DPBS pH6 and 10 mM EDTA DPBS pH6.

FIG. 9 is a graphical representation showing transduction efficiency of HEK293T cells following treatment with envelope-binding proteins (A) C2-Fc, Fc-C2 and (B) Fc-C23, C25-Fc and Fc-C33.

FIG. 10 is a graphical representation showing the effect on physical virus titre following culturing in the presence of envelope-binding proteins (A) Fc-C2 and (B) C2-Fc.

### KEY TO SEQUENCE LISTING

SEQ ID NO: 1 Amino acid sequence of Human low-density lipoprotein receptor (LDLR)

SEQ ID NO: 2 Amino acid sequence of Human LDLR complement-type repeat 2

SEQ ID NO: 3 Amino acid sequence of Human LDLR complement-type repeat 3

SEQ ID NO: 4 Amino acid sequence of Human IgG1 Fc

SEQ ID NO: 5 Ceruloplasmin (Cp) Signal peptide

SEQ ID NO: 6 HuIgG1Fc Signal peptide

SEQ ID NO: 7 Amino acid sequence of construct HuLDLR(65-105)-GS-HuIgG1Fc (including signal peptide)

SEQ ID NO: 8 Amino acid sequence of construct HuLDLR(65-144)-GS-HuIgG1Fc-1 (including signal peptide)

SEQ ID NO: 9 Amino acid sequence of construct HuIgG1Fc-GS-HuLDLR(65-105) (including signal peptide)

SEQ ID NO: 10 Amino acid sequence of construct HuIgG1Fc-GS-HuLDLR(65-144) (including signal peptide)

SEQ ID NO: 11 Amino acid sequence of construct HuLDLR[(65-105)x3]-GS-HuIgG1Fc (including signal peptide)

SEQ ID NO: 12 Amino acid sequence of construct HuLDLR[(65-105)x5]-GS-HuIgG1Fc (including signal peptide)

SEQ ID NO: 13 Amino acid sequence of construct HuLDLR[(106-144)x3]-GS-HuIgG1Fc (including signal peptide)

SEQ ID NO: 14 Amino acid sequence of construct HuIgG1Fc-GS-HuLDLR[(65-105)x3](including signal peptide)

SEQ ID NO: 15 Amino acid sequence of construct HuIgG1Fc-GS-HuLDLR[(65-105)x5](including signal peptide)

SEQ ID NO: 16 Amino acid sequence of construct HuIgG1Fc-GS-HuLDLR[(106-144)x3](including signal peptide)

SEQ ID NO: 17 Amino acid sequence of construct huLDLR(65-105)-GS-huIgG1Fc-GS-huLDLR(65-105) (including signal peptide)

SEQ ID NO: 18 Amino acid sequence of construct huLDLR(106-144)-GS-huIgG1Fc-GS-huLDLR(106-144) (including signal peptide)

SEQ ID NO: 19 Nucleic acid sequence of construct HuLDLR(65-105)-GS-HuIgG1Fc

SEQ ID NO: 20 Nucleic acid sequence of construct HuLDLR(65-144)-GS-HuIgG1Fc-1

SEQ ID NO: 21 Nucleic acid sequence of construct HuIgG1Fc-GS-HuLDLR(65-105) (intronic sequence underlined)

SEQ ID NO: 22 Nucleic acid sequence of construct HuIgG1Fc-GS-HuLDLR(65-144) (intronic sequence underlined)

SEQ ID NO: 23 Nucleic acid sequence of construct HuLDLR[(65-105)x3]-GS-HuIgG1Fc

SEQ ID NO: 24 Nucleic acid sequence of construct HuLDLR[(65-105)x5]-GS-HuIgG1Fc

SEQ ID NO: 25 Nucleic acid sequence of construct HuLDLR[(106-144)x3]-GS-HuIgG1Fc

SEQ ID NO: 26 Nucleic acid sequence of construct HuIgG1 Fc-GS-HuLDLR[(65-105)x3](intronic sequence underlined)

SEQ ID NO: 27 Nucleic acid sequence of construct HuIgG1Fc-GS-HuLDLR[(65-105)x5](intronic sequence underlined)

SEQ ID NO: 28 Nucleic acid sequence of construct HuIgG1Fc-GS-HuLDLR[(106-144)x3](intronic sequence underlined)

SEQ ID NO: 29 Nucleic acid sequence of construct huLDLR(65-105)-GS-huIgG1Fc-GS-huLDLR(65-105)

SEQ ID NO: 30 Nucleic acid sequence of construct huLDLR(106-144)-GS-huIgG1Fc-GS-huLDLR(106-144)

SEQ ID NO: 31 Amino acid sequence of Human LDLR complement-type repeat 1

SEQ ID NO: 32 Amino acid sequence of Human LDLR complement-type repeat 2

SEQ ID NO: 33 Amino acid sequence of Human LDLR complement-type repeat 3

SEQ ID NO: 34 Amino acid sequence of Human LDLR complement-type repeat 4

SEQ ID NO: 35 Amino acid sequence of Human LDLR complement-type repeat 5

SEQ ID NO: 36 Amino acid sequence of Human LDLR complement-type repeat 6

SEQ ID NO: 37 Amino acid sequence of Human LDLR complement-type repeat 7

SEQ ID NO: 38 Amino acid sequence of Human LDLR complement-type repeat 1

SEQ ID NO: 39 Amino acid sequence of Human LDLR complement-type repeat 4

SEQ ID NO: 40 Amino acid sequence of Human LDLR complement-type repeat 5

SEQ ID NO: 41 Amino acid sequence of Human LDLR complement-type repeat 6

SEQ ID NO: 42 Amino acid sequence of Human LDLR complement-type repeat 7

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, molecular biology, microbiology, virology).

Unless otherwise indicated, the conventional techniques of molecular biology, microbiology, virology, recombinant DNA technology, peptide synthesis in solution, solid phase peptide synthesis, and immunology utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The term “about”, unless stated to the contrary, refers to +/−20%, more for example +/−10%, of the designated value. For the avoidance of doubt, the term “about” followed by a designated value is to be interpreted as also encompassing the exact designated value itself (for example, “about 10” also encompasses 10 exactly).

As used herein the term “from” shall be taken to indicate that a specified integer may be obtained from a particular source albeit not necessarily directly from that source (i.e., includes recombinantly obtained).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “enveloped virus” refers to DNA and RNA viruses that have a viral envelope. Envelopes are typically derived from host cell membranes (e.g., phospholipids and proteins), but may include viral glycoproteins on the surface of the envelope. Enveloped viruses also comprise a “capsid”, which is a protein layer between the envelope and viral genome. In one example, the enveloped virus is a retrovirus. For example, the enveloped virus is a lentivirus, e.g., human immunodeficiency virus.

As used herein, the term “envelope-binding protein” refers to proteins that bind or specifically bind to a viral glycoprotein expressed on the surface of the envelope of an enveloped virus. For example, the viral glycoprotein is a VSV-G protein from Vesicular stomatitis Indiana virus (VSV).

The term “vesicular stomatitis virus G pseudotyped” or “VSV-G pseudotyped” refers to an enveloped virus comprising an envelope glycoprotein from Vesicular stomatitis Indiana virus (VSV).

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “recombinant” shall be understood to mean the product of artificial genetic recombination. A recombinant protein also encompasses a protein expressed by artificial recombinant means when it is within a cell, tissue or subject, e.g., in which it is expressed.

The term “immunoglobulin Fc” or “Fc” is used to define a C-terminal region of an immunoglobulin heavy chain that contains at least a portion of the constant region. The term includes both a native Fc and Fc variants. In some examples, a human IgG heavy chain Fc region extends from Cys226, or from Pro230, to the carboxyl-terminus of the heavy chain. However, Fcs produced by host cells may undergo post-translational cleavage of one or more, particularly one or two, amino acids from the C-terminus of the heavy chain. Thus, a Fc may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. When specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. For the purposes of nomenclature only and not limitation an exemplary sequence of a human IgG1 Fc is set forth in SEQ ID NO:4 or in Uniprot Accession No: P01857.

The term “EU numbering system of Kabat” or “EU numbering system” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a protein binds to VSV-G with materially greater affinity (e.g., 20 fold or 40 fold or 60 fold or 80 fold to 100 fold or 150 fold or 200 fold greater affinity), avidity, more readily, and/or with greater duration than it binds to other antigens. Generally, but not necessarily, reference to binding means specific binding, and each term shall be understood to provide explicit support for the other term.

An “IC₅₀ of at least” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

As used herein, the term “cell culture fluid” or “cell culture medium” will be understood to encompass the fluid or medium in which cells are grown for the purpose of producing an enveloped virus. The fluid or medium does not comprise the cells (e.g., the cells may have been removed, e.g., by centrifugation and/or removal of supernatant).

As used herein, the term “cell culture” will be understood to refer to the collective of the cell culture fluid or medium and the cultured cells.

The term “purify” or “purifying” or “purification” shall be taken to mean the removal, whether completely or partially, of at least one impurity present in the cell culture fluid, which thereby improves the level of purity of enveloped virus in solution.

The term “impurity” or “impurities” shall be taken to include one or more components in the cell culture fluid other than the enveloped virus. For example, impurities may include process related impurities such as host cell DNA, host cell proteins, and media components (e.g., fetal bovine serum).

### Envelope-Binding Proteins

### LDLR Domains

The present disclosure relates to an envelope-binding protein comprising one or more low-density lipoprotein receptor (LDLR) domains, optionally fused to a purification tag such as an immunoglobulin Fc.

As used herein, the term “LDLR” shall be understood to refer to a type I transmembrane protein which are involved in the endocytic uptake of lipoproteins in mammalian cells, as well as other members of the LDLR family such as ApoER2, VLDLR, and LRP1. The LDLR ectodomain is composed of a ligand-binding domain, an epidermal growth factor (EGF) precursor homology domain and a C-terminal domain enriched in O-linked oligosaccharides. The ligand binding domain is made of 7 cysteine-rich complement-type repeats (CR1 to CR7), each made up of approximately 40 amino acids.

For the purposes of nomenclature only and not limitation an exemplary sequence of a human LDLR is set forth in SEQ ID NO: 1 or in GenBank accession number NP_000518.

For the purposes of nomenclature only and not limitation, exemplary sequences of human LDLR CR1 to CR7 domains are set forth in SEQ ID NO: 2, 3 and 31-42. Exemplary LDLR domains CR1 to CR7 are also described by Nikolic. J., Nat Commun 9, 1029 (2018) and Kim and Bezprozvanny, Int. J. Mol. Sci. 22(9), 5030 (2021). The skilled person will appreciate that the recited CR1-7 LDLR domains may deviate from the recited amino acids without affecting the function of the protein of the disclosure. The phrase “±1 to 5 amino acids” as used herein means that the amino acid residue may deviate by 1 or 2 or 3 or 4 or 5 amino acids to the C-terminus and/or the N-terminus of the stated amino acid position.

LDLR is the main receptor of VSV-G, with the complement-type repeat domains CR2 and CR3 being primarily responsible for binding to VSV-G. LDLR mediates entry of the enveloped virus into the cell via the endosome. Following binding at neutral pH and internalization of the LDLR-VSV-G complex, VSV-G undergoes a conformational change (under acidic conditions) causing it to fuse with the endosomal membrane and release. Afterwards the LDLR is recycled back to the cell surface.

As discussed herein, VSV-G is highly toxic to stable producer cells due to its fusogenicity (i.e., cell fusion and syncytia formation) with neighbouring cells. To date, to overcome the toxicity of VSV-G in stable cell line production VSV-G has been generally expressed conditionally using e.g., Tet-inducible promoters, or transiently. Other methods have involved generating LDLR negative packaging cells that constitutively express VSV-G.

The inventors' solution to the problem of VSV-G toxicity is the generation of envelope-binding proteins that bind to VSV-G and competitively inhibit the interaction with LDLR expressed on producer cells. The proteins can subsequently be dissociated from the VSV-G under endosome-like conditions, i.e., acidic conditions. These proteins reduce toxicity of VSV-G by neutralizing VSV-G on the producer cells to reduce syncytia formation and fusogenicity, as well as reducing self-infectivity (i.e., autotransduction) of producer cells.

As disclosed herein, the proteins of the disclosure comprise one or more LDLR domains. In one example, the LDLR domain is a human LDLR domain. In one example, the one or more LDLR domains comprise a LDLR extracellular domain. For example, the LDLR extracellular domain comprises a ligand binding domain, an epidermal growth factor (EGF) precursor homology domain, and/or an O-linked sugar domain. In one example, the LDLR extracellular domain comprises a ligand binding domain. In another example, the LDLR extracellular domain comprises an epidermal growth factor (EGF) precursor homology domain. In a further example, the LDLR extracellular domain comprises an O-linked sugar domain.

In one example, the one or more LDLR domains does not comprise a cytoplasmic and/or transmembrane domain. For example, the one or more LDLR domains does not comprise a cytoplasmic domain. In one example, the one or more LDLR domains does not comprise a transmembrane domain. In a further example, the one or more LDLR domains does not comprise a cytoplasmic domain and a transmembrane domain.

In one example, the ligand binding domain comprises one or more cysteine-rich complement-type repeat (CR) domains. For example, the one or more CR domains are a CR1 domain, a CR2 domain, a CR3 domain, a CR4 domain, a CR5 domain, a CR6 domain and/or a CR7 domain. In one example, the LDLR domain is the 7 cysteine-rich complement-type repeats (CR1 to CR7). For example, the LDLR domain consists of human CR1 to CR7 LDLR domains. In one example, the LDLR domain is set forth in amino acids 25 to 313 of SEQ ID NO: 1. In another example, the LDLR domain is set forth in amino acids 23 to 313 of SEQ ID NO: 1.

In one example, the LDLR domain is a human CR1 LDLR domain. For example, the CR1 LDLR domain comprises a sequence set forth in SEQ ID NO: 31. In one example, the CR1 LDLR domain comprises a sequence set forth in SEQ ID NO: 38. In one example, a CR1 LDLR domain of the present disclosure comprises a sequence at least about 85% or 90% or 95% or 97% or 98% or 99% identical to a sequence disclosed herein.

In one example, the LDLR domain is a human CR2 LDLR domain and/or a human CR3 LDLR domain.

In one example, the protein of the disclosure comprises one or more CR2 LDLR domains and/or one or more CR3 LDLR domains.

In one example, the CR2 LDLR domain comprises a sequence set forth in SEQ ID NO: 2. In one example, the CR2 LDLR domain comprises a sequence set forth in SEQ ID NO: 32. In one example, a CR2 LDLR domain of the present disclosure comprises a sequence at least about 85% or 90% or 95% or 97% or 98% or 99% identical to a sequence disclosed herein.

In one example, the CR3 LDLR domain comprises a sequence set forth in SEQ ID NO: 3. In one example, the CR3 LDLR domain comprises a sequence set forth in SEQ ID NO: 33. In one example, a CR3 LDLR domain of the present disclosure comprises a sequence at least about 85% or 90% or 95% or 97% or 98% or 99% identical to a sequence disclosed herein.

In one example, the LDLR domain is a human CR4 LDLR domain. For example, the CR4 LDLR domain comprises a sequence set forth in SEQ ID NO: 34. In one example, the CR4 LDLR domain comprises a sequence set forth in SEQ ID NO: 39. In one example, a CR4 LDLR domain of the present disclosure comprises a sequence at least about 85% or 90% or 95% or 97% or 98% or 99% identical to a sequence disclosed herein.

In one example, the LDLR domain is a human CR5 LDLR domain. For example, the CR5 LDLR domain comprises a sequence set forth in SEQ ID NO: 35. In one example, the CR5 LDLR domain comprises a sequence set forth in SEQ ID NO: 40. In one example, a CR5 LDLR domain of the present disclosure comprises a sequence at least about 85% or 90% or 95% or 97% or 98% or 99% identical to a sequence disclosed herein.

In one example, the LDLR domain is a human CR6 LDLR domain. For example, the CR6 LDLR domain comprises a sequence set forth in SEQ ID NO: 31. In one example, the CR6 LDLR domain comprises a sequence set forth in SEQ ID NO: 41. In one example, a CR6 LDLR domain of the present disclosure comprises a sequence at least about 85% or 90% or 95% or 97% or 98% or 99% identical to a sequence disclosed herein.

In one example, the LDLR domain is a human CR7 LDLR domain. For example, the CR7 LDLR domain comprises a sequence set forth in SEQ ID NO: 31. In one example, the CR7 LDLR domain comprises a sequence set forth in SEQ ID NO: 42. In one example, a CR7 LDLR domain of the present disclosure comprises a sequence at least about 85% or 90% or 95% or 97% or 98% or 99% identical to a sequence disclosed herein.

In some examples, the protein of the disclosure comprises more than one LDLR domain. In some examples, more than one LDLR domain is linked, directly or indirectly, to a Fc. For example, a single Fc is linked to more than one LDLR domain. In some examples, the LDLR domains are linked in series, e.g., to the C-terminus of the Fc and/or the N-terminus of the Fc. In other examples, one or more LDLR domains are linked to the N-terminus of the Fc and one or more LDLR domains are linked to the C-terminus of the Fc. In some examples, the Fc can dimerize, thus further increasing the number of LDLR domains in the protein of the disclosure.

In one example, each Fc in a protein of the disclosure is linked to a single LDLR domain. For example, each Fc is linked to a single CR2 LDLR or CR3 LDLR domain.

In another example, each Fc in a protein of the disclosure is linked to two or more LDLR domains.

### Purification Tags

As described herein the envelope-binding protein comprises a purification tag.

In one example, the purification tag is a protein or peptide tag. In one example, the purification tag is a half-life extender. For example, the protein or peptide tag is selected from the group consisting of an immunoglobulin Fc and albumin. In another example, the protein or peptide tag is selected from the group consisting of a hexahistidine tag, a human influenza hemagglutinin (HA) tag, a FLAG-tag, a calmodulin binding peptide (CBP) tag, a poly-glutamate tag, a polycysteine (Cys) tag, a polyhistidine (His) tag, a Myc-tag, a streptavidin-binding peptide (SBP) tag, a streptavidin (Strep) tag, an avidin tag, a bacteriophage V5 epitope (V5) tag, an isopeptag, a SpyTag, a biotin-carboxyl carrier protein (BCCP) tag, a Halo-tag, a thioredoxin (Trx) tag, a small ubiquitin-like molecule (SUMO) tag, and a maltose binding protein (MBP) tag.

In one example, the LDLR domain is fused to a purification tag. For example, the LDLR domain is fused to an immunoglobulin Fc, albumin, a hexahistidine tag, a human influenza HA tag, a FLAG-tag, a CBP tag, a poly-glutamate tag, a Cys tag, a His tag, a Myc-tag, a SBP tag, a Strep tag, an avidin tag, a V5 tag, an isopeptag, a SpyTag, a BCCP tag, a Halo-tag, a Trx tag, a SUMO tag, or a MBP tag. For example, the LDLR domain is fused to a HA, hexahistidine, or FLAG-tag. In one example, the LDLR is fused to an immunoglobulin Fc.

Fc fusions facilitate purification of the envelope binding protein constructs with Protein A purification. The presence of the Fc also facilitates downstream affinity purification of VSVG-pseudotyped virus bound to the envelope binding protein. Fc is also dimerizable, which provides increased avidity for the envelope binding proteins.

In one example, the Fc is from IgG.

For example, the Fc is from human IgG.

For example, the Fc is from IgG1.

For example, the Fc is from human IgG1.

In certain examples, the Fc is not a full-length antibody. For example, the Fc does not comprise an antibody variable domain. In some examples, the Fc does not comprise an antibody variable domain or a C_{H}1 domain.

In one example, the Fc is a Fc variant.

Any Fc region can be modified to produce a Fc variant for use in the protein of the disclosure. As discussed herein, generally a Fc is from a human immunoglobulin. However, the Fc may be derived from an immunoglobulin of any other mammalian species, including for example, a Camelid species, a rodent (e.g. a mouse, rat, rabbit, guinea pig) or non-human primate (e.g. chimpanzee, macaque) species. Moreover, the Fc may be derived from any immunoglobulin class, including IgM, IgG. IgD, IgA and IgE, and any immunoglobulin isotype, including IgG1, IgG2, IgG3 and IgG4. In certain examples, the Fc region is an IgG Fc (e.g., a human IgG region). In certain examples, the Fc region is an IgG1 Fc region (e.g., a human IgG1). In certain examples, the Fc is a chimeric Fc comprising portions of several different Fc regions. Suitable examples of chimeric Fc regions are set forth in US20110243966. It will be appreciated that the scope of the present disclosure encompasses alleles, variants and mutations of Fc regions.

In some examples, the Fc forms a homodimer.

In other examples, the Fc forms a heterodimer. Methods of producing Fc heterodimers are known in the art (see e.g., U.S. Pat. No. 8,216,805). In some examples, the Fc is a single chain Fc, where the constituent Fes are linked together by a linker. Methods of producing single chain Fes are known in the art (see e.g., US20090252729 and US20110081345).

### Linkers

As described herein, the components of the protein of the disclosure are directly or indirectly linked to each other.

In some examples, components of the protein of the disclosure are indirectly linked, e.g., via a linker. In some examples, the linker is a polypeptide linker.

In some examples, a polypeptide linker comprises or consists of a Gly/Ser linker. As used herein, the term “Gly/Ser linker” refers to a peptide that consists of glycine and serine residues. An exemplary Gly/Ser linker comprises an amino acid sequence of the formula (Gly₄Ser)ₙ, wherein n is a positive integer (e.g., 1, 2, 3, 4, or 5). In certain examples the Gly/Ser linker is (Gly₄Ser)₁. In some examples, the Gly/Ser linker is (Gly₄Ser)₂. In some examples the Gly/Ser linker is (Gly₄Ser)₃ or (Gly₄Ser)₄ or Gly₄Ser)₅.

In some example, the linker comprises or consists of a glycine linker. An exemplary glycine (‘Gly’) linker comprises an amino acid sequence of the formula (Gly)ₙ, wherein n is a positive integer (e.g., 6 or 8). In some examples, the glycine linker is a (Gly)₈ linker or a (Gly)₆ linker.

Other linkers that are suitable for use in the proteins of the disclosure are known in the art, for example, the serine-rich linkers disclosed in U.S. Pat. No. 5,525,491, the helix forming peptide linkers (e.g., A(EAAAK)nA (n=2-5)) disclosed in Arai et al, Protein Eng 2001; 14:529-32, the stable linkers disclosed in Chen et al, Mol Pharm 2011; 8:457-65, or the rigid linkers (e.g., a (EAAAK)ₙ linker, wherein n=1, 2 or 3, a A(EAAAK)₄ALEA(EAAAK4)A linker, a AEAAAKEAAAKA linker, a (Ala-Pro)ₙ linker, wherein n=10-34, and a PAPAP linker) disclosed in Chen et al, Adv Drug Deliv Rev 2013: 65(10):1357-69.

Other exemplary linkers include GS linkers (i.e., (GS)ₙ), GGSG linkers (i.e., (GGSG)ₙ), GGGS linkers (i.e., (GGGS)ₙ wherein n is a positive integer (e.g., 1, 2, 3 or 4), GSAT linkers, SEG linkers, and GGS linkers (i.e., (GGSGGS)ₙ), wherein n is a positive integer (e.g., 1, 2, 3, 4, or 5).

Exemplary linkers of the disclosure include a GS linker, a GSGGS linker, a GGSSG linker, a GGGGS linker, and a GSGSG linker.

Polypeptide linkers of the disclosure are at least one amino acid in length and can be of varying lengths. In some examples, a polypeptide linker of the disclosure is from about 1 to about 50 amino acids in length. In another example, a polypeptide linker of the disclosure is from about 1-5 amino acids in length. In a further example, a polypeptide linker of the disclosure is from about 5-10 amino acids in length. In another example, a polypeptide linker of the disclosure is from about 10-20 amino acids in length. In another example, a polypeptide linker of the disclosure is from about 15 to about 50 amino acids in length.

### Production of Enveloped Viruses

Methods of the disclosure are applicable to producing enveloped viruses from both small- and large-scale productions. The methods are particularly useful for their ability to be scaled up for manufacturing pharmaceutical products at commercial scale.

Methods for the production of enveloped viruses will be apparent to the skilled artisan and/or described, for example, in Ansorge et al., (2010) Biochem. Eng. J. 48: 362-377; Schweizer and Merten (2010) Curr. Gene Ther. 10: 474-486; and Rodrigues et al., (2011) Viral Gene Therapy. Xu, InTech. Chapter 2: 15-40.

### Enveloped Viruses

In one example, the virus is a retrovirus, for example, a lentivirus. Exemplary retroviruses are from alpha retroviruses (such avian leukosis virus (ALV)), from beta retroviruses (such as mouse mammary tumor virus (MMTV)), from gamma retroviruses (such as murine leukemia virus (MLV)), from delta retroviruses (such as human T-lymphotropic virus (HTLV)), from epsilon retroviruses (such as Walleye dermal sarcoma virus (WDSV)), from spumavirus (such as human foamy virus (HFV) or simian foamy virus (SFV)), from primate lentiviruses such as the different types of human immunodeficiency viruses (HIV), the different types of simian immunodeficiency viruses (SIV), or from non-primate mammal lentiviruses such as the equine infectious anemia virus (EIAV), from the feline immunodeficiency virus (FIV), the caprine arthritis-encephalitis virus (CAEV), or the ovine visna-maedi virus (VMV).

### Transgene Expression

In some examples, the enveloped virus comprises a transgene introduced into its genome. The transgene will depend on the specific use for which the enveloped viral vector is intended. Exemplary transgenes include a transgene coding for a therapeutic RNA (e.g. encoding an antisense complementary RNA of a target RNA or DNA sequence), a transgene encoding for a protein that is deficient or absent in a subject affected with a pathology, or a transgene used for vaccination with DNA, i.e. a transgene coding for a protein, the expression of which will induce vaccination of the recipient body against said protein. In some examples, the transgene encodes a protein or nucleic acid useful for treating a hemoglobinopathy, e.g., sickle cell disease or a thalassemia. In some examples, the transgene encodes a protein or nucleic acid useful for treating a primary immunodeficiency. In some examples, the transgene encodes a protein or nucleic acid useful for treating Wiskott-Aldrich Syndrome. In some examples, the transgene encodes a protein or nucleic acid useful for treating X linked agammaglobulinemia.

In some examples, an enveloped virus is produced by introducing the four following elements into a host cell: an expression cassette comprising a lentiviral gene gagpol, an expression cassette comprising a lentiviral gene rev, a transgene, all positioned between a lentiviral LTR-5′ and a lentiviral LTR-3′, and an expression cassette encoding envelope glycoprotein(s).

### Producer Cell Lines

In some examples, the enveloped virus is produced from a stable line expressing one or several elements required for producing an enveloped virus (Miller (2001) Curr. Protoc. Hum. Genet. Chapter 12: Unit 12.5.; Rodrigues et al. 2011, supra). In one example, the enveloped virus is produced from a mammal host cell transfected transiently with one or several plasmids coding for the elements required for producing the virus. According to an alternative example, the elements are introduced into the cell by means of multiple plasmids: one plasmid bearing an expression cassette comprising a lentiviral gagpol gene, one plasmid bearing an expression cassette comprising a lentiviral rev gene, one plasmid bearing an expression cassette encoding the envelope glycoprotein(s), one plasmid bearing an expression cassette comprising a tetracycline transactivator (tTA) gene, and/or one plasmid bearing an expression cassette comprising a lentiviral tat gene. A transfer plasmid comprising an expression cassette with the transgene, comprised between a lentiviral LTR-5′ and LTR-3′, can be introduced as a concatemer along with a helper plasmid with an antibiotic resistance cassette to confer resistance to the producer cells.

The host cell may be selected from any cell allowing production of an enveloped virus. According to one example, the cell is selected from a human cell (HEK293, HEK293T, HEK293FT, HEK2930X, Te671, HT1080, CEM), a musteli cell (NIH-3T3), a mustelidae cell (Mpf), a canid cell (D17), and derivatives thereof. According to one example, the cell is selected from CHO cells, BHK cells, MDCK cells, C3H 10T1/2 cells, FLY I, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, B-50 cells, 3T3 cells, NIH3T3 cells, HepG2 cells, Saos-2 cells, Huh7 cells, HeLa cells, W163 cells, 211 cells, and 211 A cells, and derivatives thereof.

According to one example, the cell is selected from the GPR, GPRG, GPRT, GPRGT, and GPRTG cell lines. In another example, the cell is selected from a cell line derived from any of the above cell lines.

In one example, the enveloped virus is produced from stable producer cells. Stable producer cells can be derived from packaging cell lines, including as any of the cell lines disclosed herein. In some embodiments the packaging cell lines are GPRG or GPRTG cell lines (Throm et al. (2009) Blood 113(21):5104-5110; and Bonner et al. (2015) Molecular Therapy, Vol. 23, Suppl. 1, S35). In one example, stable producer cell line cells are generated by synthesizing a vector by cloning one or more genes into a recombinant plasmid; forming a concatemeric array from an expression cassette excised from the synthesized vector, and an expression cassette obtained from an antibiotic resistance cassette plasmid; transfecting packaging cell line cells with the formed concatemeric array; and selecting and isolating the stable producer cell line cells. Virus is produced by inducing the inducible promoters of the stable producer cell line cells.

### Cell Culture Medium

The cells are cultivated in a medium suitable for cultivation of mammal cells and for producing an enveloped virus. The cells can be cultivated in an adherent environment, e.g., while attached to a surface, or in a suspension environment, e.g., suspended in the medium. The medium may moreover be supplemented with additives known in the field such as antibiotics, serum (notably fetal calf serum, etc.) added in suitable concentrations. The medium may be supplemented with GlutaMax™ Pluronic™ F-68 (ThermoFisher), LONG® R3 IGF-I (Sigma-Aldrich), Cell Boost™ 5, and/or an anticlumping agent. The medium used may notably comprise serum or be serum-free. Culture media for mammal cells are known and include, for example, DMEM (Dulbecco's Modified Eagle's medium) medium, RPMI1640 or a mixture of various culture media, including for example DMEM/F12, or a serum-free medium like optiMEM®, optiPRO®, optiPRO-SFM®, CD293® (ThermoFisher), TransFx™ (Cytiva), BalanCD® (Irvine), Freestyle F17® (Life Technologies), or Ex-Cell® 293 (Sigma-Aldrich).

In a process using transiently transfected cells, any agent allowing transfection of plasmids may be used. Exemplary agents include calcium phosphate or polyethylencimine. The conditions (e.g., amount of plasmid(s), ratio between the plasmids, ratio between the plasmid(s) and the transfection agent, the type of medium, etc.) and the transfection time may be adapted by one skilled in the art according to the characteristics of the produced virus and/or of the transgene introduced into the transfer plasmid.

According to some examples, the culture medium used has a neutral pH (e.g. comprised between 6.8 and 7.4, notably 6.8, 6.9, 7, 7.1, 7.2, 7.3 or 7.4) conventionally used in the state of the art for cultivating cells and producing viruses. In other examples, the production process used comprises the cultivation of producing cells in a moderately acidic medium. The expression “moderately acidic condition” designates the pH of an aqueous solution comprised between 5 and 6.8, for example between 5.5 and 6.5, such as between 5.8 and 6.2. The selected pH will also depend on the buffering power of the culture medium used, which one skilled in the art may easily determine taking into account his/her general knowledge. One skilled in the art is able to modify the pH of a solution.

In one example, the production of the enveloped virus comprises: transient transfection of HEK293T cells or derivatives thereof by means of one or several plasmids coding for the elements required for production of said enveloped vector, or by the use of stable producing cells, e.g., GPRG or GPRTG, producing the vectors constitutively or after induction; culturing the cells in a suitable medium, for which the pH is of about 6 or of about 7; harvesting cell culture medium containing the enveloped virus.

### Culturing in the Presence of the Envelope-Binding Protein

The present disclosure provides methods for improving the production of enveloped virus from a cell culture. In particular, the present disclosure provides methods for producing an enveloped virus in a cell culture, the method comprising culturing a cell line in a cell culture medium comprising the envelope-binding protein of the disclosure. It will be apparent to the skilled person from the disclosure herein, that the methods of the disclosure result in increased cell quality (i.e., by reducing cell death and re-infection of cells during virus production) and virus production.

It will be apparent to the skilled person that production of an enveloped virus includes a cell expansion phase and a viral production phase.

The skilled person will understand that the cell expansion phase includes a seed train cell culture. As used herein, the term “seed train” refers to the generation of an adequate number of cells (i.e., cell growth) for viral production. The skilled person will understand that seed train cell culture comprises several cultivation systems which become larger with each passage (e.g. T-flasks, roller bottles or shake flasks, small scale bioreactor systems and subsequently larger bioreactors) in order to scale the culture from a small volume of cells to a larger volume of cells suitable for virus production.

In one example, the cells are grown in a cell expansion phase prior to virus production.

In one example, the cell expansion phase is carried out in an expansion bioreactor (also termed an N−1 bioreactor).

In one example, the viral production phase is carried out in a production bioreactor (also termed an N bioreactor).

In one example, the cell expansion phase and viral production phase are carried out in the same vessel. For example, expansion of the suspension cell line and production of the enveloped virus occur in the same vessel. For example, the cell expansion phase and viral production phase are carried out in the same bioreactor.

In one example, the cell expansion phase and viral production phase are carried out in different vessels. For example, the cell expansion phase is carried out in an expansion bioreactor and viral production phase is carried out in a production bioreactor, wherein the expansion bioreactor and the production bioreactor are different.

In one example, the cell culture is operated in a batch, fed batch, continuous, semi-continuous, or perfusion mode.

In one example, the cell expansion phase and/or viral production phase are operated in a batch, fed batch, continuous, semi-continuous, or perfusion mode.

In one example, the cell expansion phase is carried out in batch, fed batch, continuous, semi-continuous, or perfusion mode. In one example, the cell expansion phase is carried out in batch mode. In another example, the cell expansion phase is carried out in fed-batch mode. In a further example, the cell expansion phase is carried out in continuous mode. In one example, the cell expansion phase is carried out in perfusion mode. In another example, the cell expansion phase is carried out in batch and perfusion mode. For example, the cell expansion phase is initially carried out in batch mode and subsequently carried out in perfusion mode.

In one example, the viral production phase is carried out in batch, fed batch, continuous, semi-continuous, or perfusion mode. In one example, the viral production phase is carried out in batch mode. In another example, the viral production phase is carried out in fed-batch mode. In a further example, the viral production phase is carried out in continuous mode. In one example, the viral production phase is carried out in perfusion mode. In another example, the viral production phase is carried out in batch and perfusion mode. For example, the viral production phase is initially carried out in batch mode and subsequently carried out in perfusion mode.

In one example, the cell expansion and the viral production phases are carried out in batch mode. In another example, the cell expansion and the viral production phases are carried out in perfusion mode. In a further example, the cell expansion phase is carried out in batch mode and the viral production phase is carried out in perfusion mode.

It will be apparent to the skilled person that reference to a batch, fed-batch, continuous and/or perfusion mode for a particular phase of cell culture (i.e., cell expansion and/or viral production) does not mean that the entire culture phase is carried out in that mode. For example, it only means that a period of the cell culture phase (e.g., at least 1 day) is carried out in that mode. It will also be understood that the mode does not necessary commence on day 0 of the culture phase. For example, the culture may commenced on day 0 and perfusion mode only commenced on day 2 of the cell culture phase.

In one example, the suspension cell culture is operated in batch mode. It will be apparent to the skilled person that “batch mode” refers to a process where cells are initially cultured in a medium and this medium is neither removed, replaced, nor supplemented, i.e., the cells are not “fed” with new medium, during or before the end of cultivation.

In one example, the suspension cell culture is operated in fed-batch mode. It will be apparent to the skilled person that “fed-batch mode” refers to a process where one or more nutrients are fed to the bioreactor during the cultivation period. In one example, the cell expansion phase and/or the virus production phase are operated in fed-batch mode. In one example, the cell expansion phase is operated in fed-batch mode.

In one example, the suspension cell culture is operated in perfusion mode. It will apparent to the skilled person that “perfusion mode” involves the constant feeding of fresh media and removal of spent media while retaining high numbers of viable cells (i.e., continuous media exchange). In one example, the cell expansion phase and/or the virus production phase are operated in perfusion mode. In one example, the virus production phase is operated in perfusion mode.

In one example of any method described herein, the cells are cultured in a cell expansion phase in the absence of the envelope-binding protein of the disclosure, followed a virus production phase in the presence of the envelope-binding protein of the disclosure.

In one example, the envelope-binding protein is added to the cell culture medium during the virus production phase.

In one example, the envelope-binding protein is added as an excipient to the cell culture medium.

It will be apparent to the skilled person from the disclosure herein that the envelope-binding protein is added to the cell culture medium as a single bolus feed, as multiple feeds, or continuously over the duration of the culture. In one example, the envelope-binding protein is added to the cell culture medium as a single bolus feed. For example, the envelope-binding protein is added to the cell culture medium at the start of the virus production phase of the cell culture (i.e., Induction Day 0). In another example, the envelope-binding protein is added to the cell culture medium every day or every second day for the duration of the virus production phase of the cell culture. In one example, the envelope-binding protein is added to the cell culture medium by perfusion cell culture at a defined concentration. For example, the envelope-binding protein is added to the cell culture medium via perfusion cell culture at a concentration of at least 0.001 μg/mL. In a further example, the envelope-binding protein is added to the cell culture medium as required to maintain a minimum concentration of envelope-binding protein in the cell culture medium. For example, the envelope-binding protein is added to the cell culture medium to maintain a concentration of at least 0.001 μg/mL in the cell culture medium.

In one example, the cell line is a cell line expressing a “tetracycline-suppressible gene expression system” or “Tet-OFF” system. As used herein, reference to a “tetracycline-suppressible gene expression system” or “Tet-OFF” system refers to cell line that stably expresses tetracycline-controlled transactivator (tTA) such that the presence of tetracycline or a derivative thereof (e.g., doxycycline) silences transcription from tetracycline responsive element promoters.

Accordingly, the skilled person will understand that initially cells are cultured in the presence of tetracycline or a derivative thereof to suppress virus production but allow cell growth or expansion of the cell line, followed by which the tetracycline or a derivative thereof is removed from the cell culture to permit virus production.

In one example of the disclosure herein, the cell line co-expresses the envelope-binding protein of the disclosure. For example, the cell line expresses a Tet-Off system and the envelope-binding protein of the disclosure. It will be apparent to the skilled person that according to this example, expression of the envelope-binding protein of the disclosure will be under the control of the Tet-Off system and as such, withdrawal (or a reduction in the concentration) of tetracycline or a derivative results in expression of the enveloped virus, and expression of the envelope-binding protein of the disclosure.

Methods of removing tetracycline or a derivative thereof will be apparent to the skilled person and/or described herein. For example, methods of removing tetracycline or derivatives thereof known in the art include centrifuging cells, removing supernatant and subsequently resuspending cells in tetracycline-free media.

In one example, the cells are cultured in fluidized bed bioreactors, hollow fiber bioreactors, roller bottles, shake flasks, or stirred tank bioreactors.

In one example, the volume of the cell culture can be for example, about 0.01 L to about 0.1 L, or about 0.1 L to about 1 L, or about 1 L to about 5 L. In another example, the volume of the cell culture can be about 5 L to about 10 L, about 10 L to about 50 L, about 50 L to about 100 L, about 100 L to about 200 L, about 200 L to about 500 L, about 500 L to about 1000 L, about 1000 L to about 2000 L, or about 2000 L to about 5000 L. In one example, the volume of the cell culture is between about 35 and 150 L. In one example, the volume of the cell culture is about 35-150 L. In one example, the volume of the cell culture is about 50-70 L.

In one example, the cell culture is operated at a temperature that permits cell growth and viral production. For example, the cell culture has a temperature conventionally used in the state of the art for cultivating cells and producing viruses. In one example, the cell culture is at a temperature of between 35-39° C. For example, at a temperature of 37±0.5° C. or at a temperature of 38±0.5° C.

### Purifying Enveloped Viruses

In one example, the enveloped virus is purified from the cell culture comprising one or more steps selected from the group consisting of clarification filtration, affinity chromatography, anion exchange chromatography, concentration and diafiltration.

The downstream process for purifying and concentrating viral vector from a cell culture includes a harvest filtration step (also known as “clarification filtration” or “harvest clarification filtration” or “bioburden reduction”) to remove cellular debris and components from the harvest, a purification step, e.g., anion exchange chromatography, to reduce overall volume and to separate viral vector from host cell DNA, proteins, and media components, and an ultrafiltration/diafiltration step to concentrate the viral vector into a final formulation buffer. In some examples, the downstream step further includes a sterile filtration step for removal of microorganisms from the final product.

As used herein, “harvesting” refers to removal of the cell culture media containing virus particles from the producer cells for downstream processing, and “harvest” refers to the cell culture media containing virus particles that has been removed for the purpose of downstream processing. A harvesting process may include collecting one or more harvests. “Harvest filtration” refers to either a harvest that has been filtered or cell culture media containing virus particles that has been filtered to remove the producer cells for downstream processing.

In one example, a harvested cell culture fluid is filtered following production of the enveloped virus.

As used herein, the term “filtered cell culture fluid” will be understood to encompass the cell culture fluid after it has been subjected to harvest filtration.

Following harvest filtration, the enveloped virus is purified. In one example, the enveloped virus is purified using chromatography. In one example, the enveloped virus is purified using ion exchange chromatography. In one example, the enveloped virus is purified using affinity capture chromatography.

In one example, the affinity chromatography is protein A chromatography.

In one example, the affinity chromatography step comprises an affinity chromatography resin.

In one example, the present disclosure provides a method of affinity chromatography, the method comprising binding the enveloped virus to an affinity chromatography resin and collecting the enveloped virus, wherein the enveloped virus is bound to an envelope-binding protein of the disclosure.

In one example, the method comprises (i) loading the filtered cell culture fluid comprising the enveloped virus onto an affinity chromatography resin and (ii) collecting the enveloped virus, wherein the enveloped virus is bound to an envelope-binding protein of the disclosure.

In one example, the affinity chromatography step comprises an affinity chromatography resin comprising a ligand comprising an envelope-binding protein of the disclosure. For example, the affinity chromatography resin comprises an envelope-binding protein of the disclosure immobilized to a matrix of the affinity chromatography resin.

In one example, the present disclosure provides a method of affinity chromatography, the method comprising binding the enveloped virus to an affinity chromatography resin comprising an envelope-binding protein of the disclosure and collecting the enveloped virus.

In one example, the method comprises (i) loading the filtered cell culture fluid comprising the enveloped virus onto an affinity chromatography resin comprising an envelope-binding protein of the disclosure and (ii) collecting the enveloped virus.

It will be apparent to the skilled person from the disclosure herein that the enveloped virus is eluted from the affinity chromatography resin by altering the pH and/or concentration of calcium in the elution buffer. For example, the enveloped virus is eluted from the affinity chromatography resin by altering the pH of the elution buffer.

For example, the pH is decreased below a pH 6.0 to increase elution of the enveloped virus from the affinity chromatography resin. In one example, the enveloped virus is eluted from the affinity chromatography resin by altering the concentration of calcium in the elution buffer. For example, the concentration of calcium is decreased to increase elution of the enveloped virus from the affinity chromatography resin.

In one example, the enveloped virus is further purified using one or more additional purification steps. Methods of purifying the enveloped virus will be apparent to the skilled person and/or are described herein. In one example, purifying the enveloped virus comprises one or more steps selected from the group consisting of clarification filtration, anion exchange chromatography, concentration and diafiltration.

In one example, the enveloped virus is further purified using anion exchange chromatography. For example, the anion exchange is performed in bind-elute mode. In this regard, the enveloped virus binds to the anion exchanger while contaminants flow through. The virus is subsequently eluted from the anion exchanger. Performing anion exchange in this manner reduces the volume of liquid in which the virus is suspended and removes contaminants such as host cell DNA, host cell proteins, and medium components like fetal bovine serum.

Suitable anion exchangers will be apparent to the skilled artisan. Exemplary anion exchangers are a column comprising a resin or a membrane or another suitable substrate.

In one example, the anion exchanger is a weak anion exchanger, e.g., comprising an ion exchange group selected from a diethylaminoethyl (DEAE) or aminoethyl group.

In another example, the anion exchanger is a strong anion exchanger, e.g., comprising an ion exchange group selected from a quaternary ammonium (Q), diethyl-2-hydroxypropylaminoethyl (QAE), triethylaminoethyl (TEAE), or trimethyl aminoethyl group. Exemplary anion exchangers include MUSTANG® E, MUSTANG® Q, SARTOBIND® Q, CHROMASORB®, POSSIDYNE®, CAPTO® Q, QSFF, POROS® Q, FRACTOGEL® Q, NATRIX® Q.

In one example, the anion exchanger comprises a Q ion exchange group.

In one example, the anion exchanger is a membrane anion exchanger comprising a Q ion exchange group. For example, the anion exchanger is MUSTANG® Q.

In one example, an enveloped virus eluted from anion exchange column is further purified on the basis of its size. In one example, the buffer in which virus was eluted from the anion exchange column, is exchanged more or less at the same time. In the process of the disclosure, tangential flow filtration is preferred. This method permits impurity removal and buffer exchange at almost the same time.

Tangential flow ultrafiltration/diafiltration is a method which may be used to remove residual protein and nucleic acids as well as for exchanging working buffer into a final formulation buffer. Ultrafiltration using tangential flow is preferred and different devices can be used (e.g. Proflux and LABSCALE (ultrafiltration system) TFF System, both Millipore or the KR2i system from Repligen). The particular ultrafiltration membrane selected will be of a filter pore size sufficient small to retain enveloped virus but large enough to allow penetration of impurities. Depending on the manufacturer and membrane type, nominal molecular weight cut-offs between 100 and 1000 kDa may be appropriate (e.g. UFP-750-E-5A, GE Healthcare: BIOMAX (ultrafiltration device) NMWC 1000, Millipore). In one example, the molecular weight cut-off is 500 kDa. The membrane composition may be, but it is not limited to, regenerate cellulose, (modified) polyethersulfone, polysulfone. Membranes can be of flat sheet or hollow fibre type. The main parameters that must be optimized are flux rate and trans-membrane pressure. In combination with nominal molecular weight cut-off these two parameters will enable efficient purification and buffer exchange and high virus yield.

As an additional step sterile filtration may be performed to eliminate bioburden. Therefore diluted eluate or final retentate from the ultrafiltration step may be filtered through a filter, for example a 0.22 μm filter. The filter may be constructed from various materials, which may include but are not limited to polypropylene, hydrophilic PVDF, cellulose, hydrophilic regenerated cellulose, cellulose esters, wetting agent-free cellulose acetate, cellulose acetate, nylon, hydrophilic nylon membrane, polyethersulfone, hydrophilic polyethersulfone, hydrophilic asymmetric PES, or any other material which is consistent with low unspecific influenza virus binding. The filter may have a single membrane layer or more than one layer or may incorporate a prefilter of the same or different material, for example a 0.45 μm prefilter. The sterile filtrated virus can be held frozen for subsequent manipulation.

The invention is further disclosed in the following numbered paragraphs:
1. An envelope-binding protein comprising one or more low-density lipoprotein receptor (LDLR) domains and a purification tag.
2. The envelope-binding protein of paragraph 1, wherein the envelope-binding protein specifically binds vesicular stomatitis virus G (VSV-G) pseudotyped virus.
3. The envelope-binding protein of paragraphs 1 or 2, wherein the envelope-binding protein specifically binds VSV-G pseudotyped virus in the presence of calcium.
4. The envelope-binding protein of any one of paragraphs 1 to 3, wherein the envelope-binding protein specifically binds VSV-G pseudotyped virus in the presence of at least about 0.01 mM calcium.
5. The envelope-binding protein of any one of paragraphs 1 to 4, wherein the envelope-binding protein specifically binds VSV-G pseudotyped virus in the presence of at least about 5 mM calcium.
6. The envelope-binding protein of paragraph 5, wherein the envelope-binding protein binds VSV-G at a pH of between pH 6.5 and pH 9.0.
7. The envelope-binding protein of paragraphs 5 or 6, wherein binding of the envelope-binding protein to VSV-G dissociates at pH below 6.5.
8. The envelope-binding protein of any one of paragraphs 1 to 7, wherein the one or more LDLR domains comprise a LDLR extracellular domain.
9. The envelope-binding protein of paragraph 8, wherein the LDLR extracellular domain comprises a ligand binding domain, an epidermal growth factor (EGF) precursor homology domain, and/or an O-linked sugar domain.
10. The envelope-binding protein of paragraph 9, wherein the ligand binding domain comprises one or more cysteine-rich complement-type repeat (CR) domains.
11. The envelope-binding protein of paragraph 10, wherein the one or more CR domains are a CR1 domain, a CR2 domain, a CR3 domain, a CR4 domain, a CR5 domain, a CR6 domain and/or a CR7 domain.
12. The envelope-binding protein of any one of paragraphs 1 to 11, wherein the envelope-binding protein comprises two or more LDLR domains directly or indirectly linked to each other.
13. The envelope-binding protein of paragraph 12, wherein the two or more LDLR domains are indirectly linked to each other via a linker.
14. The envelope-binding protein of paragraph 13, wherein the linker is a peptide linker comprising at least 2 amino acids in length.
15. The envelope-binding protein of paragraphs 13 or 14, wherein the linker is selected from the group consisting of a GS linker, a GSGGS linker, a GGSSG linker, a GGGGS linker, a GSGSG linker, a (Gly)₈ linker, a (Gly)₆ linker, a (GGGS)ₙ linker, wherein n=1, 2, 3 or 4, a (EAAAK)ₙ linker, wherein n=1, 2 or 3, a A(EAAAK)₄ALEA(EAAAK₄)A linker, a AEAAAKEAAAKA linker, a (Ala-Pro)ₙ linker, wherein n=10-34, and a PAPAP linker.
16. The envelope-binding protein of any one of paragraphs 1 to 15, wherein the purification tag is directly linked to the one or more LDLR domains.
17. The envelope-binding protein of any one of paragraphs 1 to 16, wherein the purification tag is indirectly linked to the one or more LDLR domains via a linker.
18. The envelope-binding protein of paragraph 17, wherein the linker is a peptide linker comprising at least 2 amino acids in length.
19. The envelope-binding protein of paragraphs 17 or 18, wherein the linker is selected from the group consisting of a GS linker, a GSGGS linker, a GGSSG linker, a GGGGS linker, a GSGSG linker, a (Gly)₈ linker, a (Gly)₆ linker, a (GGGS)ₙ linker, wherein n=1, 2, 3 or 4, a (EAAAK)ₙ linker, wherein n=1, 2 or 3, a A(EAAAK)₄ALEA(EAAAK₄)A linker, a AEAAAKEAAAKA linker, a (Ala-Pro)ₙ linker, wherein n=10-34, and a PAPAP linker.
20. The envelope-binding protein of any one of paragraphs 1 to 19, wherein the envelope-binding protein comprises, in order from N- to C-terminus:
   (i) a LDLR domain indirectly or directly linked to the purification tag;
   (ii) the purification tag indirectly or directly linked to a LDLR domain; or
   (iii) a first LDLR domain, the purification tag, and a second LDLR domain, wherein each of the first and/or second LDLR domains and the purification tag are indirectly or directly linked.
21. The envelope-binding protein of paragraph 20, wherein the envelope-binding protein comprises one or more additional LDLR domains indirectly or directly linked to the N- and/or C-terminus.
22. The envelope-binding protein of any one of paragraphs 1 to 21, wherein the one or more LDLR domains are a complement-type repeat 1 (CR1) LDLR domain, a CR2 LDLR domain, a CR3 LDLR domain, a CR4 LDLR domain, a CR5 LDLR domain, a CR6 LDLR domain and/or a CR7 LDLR domain.
23. The envelope-binding protein of any one of paragraphs 1 to 22, wherein the one or more LDLR domains are a complement-type repeat 2 (CR2) LDLR domain and/or a CR3 LDLR domain.
24. The envelope-binding protein of paragraphs 22 or 23, wherein the envelope-binding protein comprises five or fewer CR2 LDLR domains; and/or five or fewer CR3 LDLR domains.
25. The envelope-binding protein of any one of paragraphs 22 to 24, wherein the envelope-binding protein comprises, in order from N- to C-terminus:
   (i) a CR2 LDLR domain indirectly or directly linked to the purification tag;
   (ii) a CR3 LDLR domain indirectly or directly linked to the purification tag;
   (iii) the purification tag indirectly or directly linked to a CR2 LDLR domain;
   (iv) the purification tag indirectly or directly linked to a CR3 LDLR domain;
   (v) a first CR2 LDLR domain, the purification tag and a second CR2 LDLR domain, wherein each of the CR2 LDLR domains and the purification tag are indirectly or directly linked;
   (vi) a first CR3 LDLR domain, the purification tag and a second CR3 LDLR domain, wherein each of the CR3 LDLR domains and the purification tag are indirectly or directly linked;
   (vii) a CR2 LDLR domain, the purification tag and a CR3 LDLR domain, wherein the CR2 and CR3 LDLR domains and the purification tag are indirectly or directly linked; or
   (viii) a CR3 LDLR domain, the purification tag and a CR2 LDLR domain, wherein the CR2 and CR3 LDLR domains and the purification tag are indirectly or directly linked.
26. The envelope-binding protein of paragraph 25, wherein the envelope-binding protein comprises one or more additional CR2 and/or CR3 LDLR domains indirectly or directly linked to the N- and/or C-terminus.
27. The envelope-binding protein of paragraph 22, wherein the envelope-binding protein comprises a CR1 LDLR domain, a CR2 LDLR domain, a CR3 LDLR domain, a CR4 LDLR domain, a CR5 LDLR domain, a CR6 LDLR domain and a CR7 LDLR domain.
28. The envelope-binding protein of paragraph 27, wherein the envelope-binding protein comprises, in order from N- to C-terminus:
   (i) a CR1 LDLR domain, a CR2 LDLR domain, a CR3 LDLR domain, a CR4 LDLR domain, a CR5 LDLR domain, a CR6 LDLR domain and a CR7 LDLR domain indirectly or directly linked to the purification tag; or
   (ii) the purification tag indirectly or directly linked to a CR1 LDLR domain, a CR2 LDLR domain, a CR3 LDLR domain, a CR4 LDLR domain, a CR5 LDLR domain, a CR6 LDLR domain and a CR7 LDLR domain.
29. The envelope-binding protein of any one of paragraphs 1 to 28, wherein the purification tag is a protein or peptide tag.
30. The envelope-binding protein of paragraph 29, wherein the protein or peptide tag is selected from the group consisting of an immunoglobulin Fc, albumin, a hexahistidine tag, a human influenza hemagglutinin (HA) tag, a FLAG-tag, a calmodulin binding peptide (CBP) tag, a poly-glutamate tag, a polycysteine (Cys) tag, a polyhistidine (His) tag, a Myc-tag, a streptavidin-binding peptide (SBP) tag, a streptavidin (Strep) tag, an avidin tag, a bacteriophage V5 epitope (V5) tag, an isopeptag, a SpyTag, a biotin-carboxyl carrier protein (BCCP) tag, a Halo-tag, a thioredoxin (Trx) tag, a small ubiquitin-like molecule (SUMO) tag, and a maltose binding protein (MBP) tag.
31. The envelope-binding protein of paragraphs 29 or 30, wherein the protein tag comprises an immunoglobulin Fc.
32. The envelope-binding protein of paragraphs 30 or 31, wherein the envelope-binding protein comprises, in order from N- to C-terminus:
   (i) a CR2 or CR3 LDLR domain indirectly or directly linked to the immunoglobulin Fc; or
   (ii) the immunoglobulin Fc indirectly or directly linked to a CR2 or CR3 LDLR domain; or
   (iii) a CR2 or CR3 LDLR domain, the immunoglobulin Fc, and a second CR2 or CR3 LDLR domain, wherein each of the CR2 and/or CR3 LDLR domains and the immunoglobulin Fc are indirectly or directly linked.
33. The envelope-binding protein of any one of paragraphs 30 to 32, wherein the envelope-binding protein comprises, in order from N- to C-terminus:
   (i) a CR2 LDLR domain indirectly or directly linked to the immunoglobulin Fc;
   (ii) a CR3 LDLR domain indirectly or directly linked to the immunoglobulin Fc;
   (iii) the immunoglobulin Fc indirectly or directly linked to a CR2 LDLR domain;
   (iv) the immunoglobulin Fc indirectly or directly linked to a CR3 LDLR domain;
   (v) a first CR2 LDLR domain, the immunoglobulin Fc and a second CR2 LDLR domain, wherein each of the CR2 LDLR domains and the immunoglobulin Fc are indirectly or directly linked;
   (vi) a first CR3 LDLR domain, the immunoglobulin Fc and a second CR3 LDLR domain, wherein each of the CR3 LDLR domains and the immunoglobulin Fc are indirectly or directly linked;
   (vii) a CR2 LDLR domain, the immunoglobulin Fc and a CR3 LDLR domain, wherein the CR2 and CR3 LDLR domains and the immunoglobulin Fc are indirectly or directly linked; or
   (viii) a CR3 LDLR domain, the immunoglobulin Fc and a CR2 LDLR domain, wherein the CR2 and CR3 LDLR domains and the immunoglobulin Fc are indirectly or directly linked.
34. The envelope-binding protein of paragraphs 32 or 33, wherein the envelope-binding protein comprises one or more additional CR2 and/or CR3 LDLR domains indirectly or directly linked to the N- and/or C-terminus.
35. The envelope-binding protein of any one of paragraphs 30 to 34, wherein the envelope-binding protein comprises, in order from N- to C-terminus:
   (i) a CR2 LDLR domain indirectly or directly linked to the immunoglobulin Fc;
   (ii) a CR2 LDLR domain, a CR3 LDLR domain and the immunoglobulin Fc, wherein the CR2 and CR3 LDLR domains and the immunoglobulin Fc are indirectly or directly linked;
   (iii) the immunoglobulin Fc indirectly or directly linked to a CR2 LDLR domain;
   (iv) the immunoglobulin Fc, a CR2 LDLR domain, a CR3 LDLR domain, wherein the immunoglobulin Fc and the CR2 and CR3 LDLR domains are indirectly or directly linked;
   (v) a first CR2 LDLR domain, a second CR2 LDLR domain, a third CR2 LDLR domain and the immunoglobulin Fc, wherein the CR2 LDLR domains and the immunoglobulin are indirectly or directly linked;
   (vi) a first CR2 LDLR domain, a second CR2 LDLR domain, a third CR2 LDLR domain, a fourth CR2 LDLR domain, a fifth CR2 LDLR domain and the immunoglobulin Fc, wherein the CR2 LDLR domains and the immunoglobulin are indirectly or directly linked;
   (vii) a first CR3 LDLR domain, a second CR3 LDLR domain, a third CR3 LDLR domain and the immunoglobulin Fc, wherein the CR3 LDLR domains and the immunoglobulin are indirectly or directly linked;
   (viii) the immunoglobulin Fc, a first CR2 LDLR domain, a second CR2 LDLR domain, and a third CR2 LDLR domain, wherein the immunoglobulin and the CR2 LDLR domains are indirectly or directly linked;
   (ix) the immunoglobulin Fc, a first CR2 LDLR domain, a second CR2 LDLR domain, a third CR2 LDLR domain, a fourth CR2 LDLR domain and a fifth CR2 LDLR domain, wherein the immunoglobulin and the CR2 LDLR domains are indirectly or directly linked;
   (x) the immunoglobulin Fc, a first CR3 LDLR domain, a second CR3 LDLR domain, and a third CR3 LDLR domain, wherein the immunoglobulin and the CR3 LDLR domains are indirectly or directly linked;
   (xi) a first CR2 LDLR domain, the immunoglobulin Fc, a second CR2 LDLR domain, wherein the immunoglobulin and the CR2 LDLR domains are indirectly or directly linked; or
   (xii) a first CR3 LDLR domain, the immunoglobulin Fc, a second CR3 LDLR domain, wherein the immunoglobulin and the CR3 LDLR domains are indirectly or directly linked.
36. The envelope-binding protein of paragraph 35, wherein the envelope-binding protein comprises one or more additional CR2 and/or CR3 LDLR domains indirectly or directly linked to the N- and/or C-terminus.
37. The envelope-binding protein of any one of paragraphs 22 to 36, wherein the CR2 LDLR domain comprises a sequence set forth in SEQ ID NO: 2 and/or the CR3 LDLR domain comprises a sequence set forth in SEQ ID NO: 3.
38. The envelope-binding protein of any one of paragraphs 30 to 37, wherein the immunoglobulin Fc is an IgG1 Fc.
39. The envelope-binding protein of paragraph 38, wherein the IgG1 Fc comprises a sequence set forth in SEQ ID NO: 4.
40. The envelope-binding protein of any one of paragraphs 30 to 39, wherein the envelope-binding protein comprises, or consists of, in order from N- to C-terminus:
   (i) a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4;
   (ii) a CR2 LDLR domain comprising amino acids 65 (±1 to 5 amino acids) to 105 (±1 to 5 amino acids) of SEQ ID NO: 1, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4;
   (iii) the immunoglobulin Fc comprising a sequence set forth in SEQ ID NO: 4, a GS linker and a CR2 LDLR domain comprising amino acids 65 (±1 to 5 amino acids) to 105 (±1 to 5 amino acids) of SEQ ID NO: 1;
   (iv) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1 and a CR3 LDLR domain comprising amino acid residues 106 (±1 to 5 amino acids) to 144 (±1 to 5 amino acids) of SEQ ID NO: 1;
   (v) a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GS linker and the immunoglobulin Fc comprising a sequence set forth in SEQ ID NO: 4;
   (vi) a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker, a CR2 LDLR domain comprising amino acids 65 (±1 to 5 amino acids) to 105 (±1 to 5 amino acids) of SEQ ID NO: 1 and the immunoglobulin Fc comprising a sequence set forth in SEQ ID NO: 4;
   (vii) a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, a CR3 LDLR domain comprising amino acid residues 106 (±1 to 5 amino acids) to 144 (±1 to 5 amino acids) of SEQ ID NO: 1, a GS linker and the immunoglobulin Fc comprising a sequence set forth in SEQ ID NO: 4;
   (viii) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker and a CR2 LDLR domain comprising amino acids 65 (±1 to 5 amino acids) to 105 (±1 to 5 amino acids) of SEQ ID NO: 1;
   (ix) the immunoglobulin Fc comprising a sequence set forth in SEQ ID NO: 4, a GS linker, a CR2 LDLR domain comprising amino acids 65 (±1 to 5 amino acids) to 105 (±1 to 5 amino acids) of SEQ ID NO: 1, a GSGGS linker, a CR2 LDLR domain comprising amino acids 65 (±1 to 5 amino acids) to 105 (±1 to 5 amino acids) of SEQ ID NO: 1, a GGSSG linker, a CR2 LDLR domain comprising amino acids 65 (±1 to 5 amino acids) to 105 (±1 to 5 amino acids) of SEQ ID NO: 1, a GGGGS linker, a CR2 LDLR domain comprising amino acids 65 (±1 to 5 amino acids) to 105 (±1 to 5 amino acids) of SEQ ID NO: 1, a GSGSG linker and a CR2 LDLR domain comprising amino acids 65 (±1 to 5 amino acids) to 105 (±1 to 5 amino acids) of SEQ ID NO: 1;
   (x) the immunoglobulin Fc comprising a sequence set forth in SEQ ID NO: 4, a GS linker, a CR3 LDLR domain comprising amino acid residues 106 (±1 to 5 amino acids) to 144 (±1 to 5 amino acids) of SEQ ID NO: 1, a GSGGS linker, a CR3 LDLR domain comprising amino acid residues 106 (±1 to 5 amino acids) to 144 (±1 to 5 amino acids) of SEQ ID NO: 1, a GGSSG linker, and a CR3 LDLR domain comprising amino acid residues 106 (±1 to 5 amino acids) to 144 (±1 to amino acids) of SEQ ID NO: 1;
   (xi) a CR2 LDLR domain comprising amino acids 65 (±1 to 5 amino acids) to 105 (±1 to 5 amino acids) of SEQ ID NO: 1, a GS linker, the immunoglobulin Fc comprising a sequence set forth in SEQ ID NO: 4, a GS linker, and a CR2 LDLR domain comprising amino acids 65 (±1 to 5 amino acids) to 105 (±1 to 5 amino acids) of SEQ ID NO: 1; or
   (xii) a CR3 LDLR domain comprising amino acid residues 106 (±1 to 5 amino acids) to 144 (±1 to 5 amino acids) of SEQ ID NO: 1, a GS linker, the immunoglobulin Fc comprising a sequence set forth in SEQ ID NO: 4, a GS linker, and a CR3 LDLR domain comprising amino acid residues 106 (±1 to 5 amino acids) to 144 (±1 to 5 amino acids) of SEQ ID NO: 1.
41. The envelope-binding protein of any one of paragraphs 30 to 40, wherein the envelope-binding protein comprises, in order from N- to C-terminus:
   (i) a CR2 LDLR domain comprising a sequence set forth in SEQ ID NO: 2, a GS linker and the immunoglobulin Fc comprising a sequence set forth in SEQ ID NO: 4;
   (ii) a CR2 LDLR domain comprising a sequence set forth in SEQ ID NO: 2, a CR3 LDLR domain comprising a sequence set forth in SEQ ID NO: 3, a GS linker and the immunoglobulin Fc comprising a sequence set forth in SEQ ID NO: 4;
   (iii) the immunoglobulin Fc comprising a sequence set forth in SEQ ID NO: 4, a GS linker and a CR2 LDLR domain comprising a sequence set forth in SEQ ID NO: 2;
   (iv) the immunoglobulin Fc comprising a sequence set forth in SEQ ID NO: 4, a GS linker, a CR2 LDLR domain comprising a sequence set forth in SEQ ID NO: 2 and a CR3 LDLR domain comprising a sequence set forth in SEQ ID NO: 3;
   (v) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GS linker and the immunoglobulin Fc comprising a sequence set forth in SEQ ID NO: 4;
   (vi) a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker, a CR2 LDLR domain comprising a sequence set forth in SEQ ID NO: 2 and the immunoglobulin Fc comprising a sequence set forth in SEQ ID NO: 4;
   (vii) a CR3 LDLR domain comprising a sequence set forth in SEQ ID NO: 3, a GSGGS linker, a CR3 LDLR domain comprising a sequence set forth in SEQ ID NO: 3, a GGSSG linker, a CR3 LDLR domain comprising a sequence set forth in SEQ ID NO: 3, a GS linker and the immunoglobulin Fc comprising a sequence set forth in SEQ ID NO: 4;
   (viii) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker and a CR2 LDLR domain comprising a sequence set forth in SEQ ID NO: 2;
   (ix) the immunoglobulin Fc comprising a sequence set forth in SEQ ID NO: 4, a GS linker, a CR2 LDLR domain comprising a sequence set forth in SEQ ID NO: 2, a GSGGS linker, a CR2 LDLR domain comprising a sequence set forth in SEQ ID NO: 2, a GGSSG linker, a CR2 LDLR domain comprising a sequence set forth in SEQ ID NO: 2, a GGGGS linker, a CR2 LDLR domain comprising a sequence set forth in SEQ ID NO: 2, a GSGSG linker and a CR2 LDLR domain comprising a sequence set forth in SEQ ID NO: 2;
   (x) the immunoglobulin Fc comprising a sequence set forth in SEQ ID NO: 4, a GS linker, a CR3 LDLR domain comprising a sequence set forth in SEQ ID NO: 3, a GSGGS linker, a CR3 LDLR domain comprising a sequence set forth in SEQ ID NO: 3, a GGSSG linker, and a CR3 LDLR domain comprising a sequence set forth in SEQ ID NO: 3;
   (xi) a CR2 LDLR domain comprising a sequence set forth in SEQ ID NO: 2, a GS linker, the immunoglobulin Fc comprising a sequence set forth in SEQ ID NO: 4, a GS linker, and a CR2 LDLR domain comprising a sequence set forth in SEQ ID NO: 2; or
   (xii) a CR3 LDLR domain comprising a sequence set forth in SEQ ID NO: 3, a GS linker, the immunoglobulin Fc comprising a sequence set forth in SEQ ID NO: 4, a GS linker, and a CR3 LDLR domain comprising a sequence set forth in SEQ ID NO: 3.
42. The envelope-binding protein of any one of paragraphs 1 to 41, wherein the envelope-binding protein comprises or consists of a sequence set forth in any one of SEQ ID NOs: 7 to 18.
43. The envelope-binding protein of any one of paragraphs 1 to 42, wherein the envelope-binding protein comprises or consists of a sequence expressed from or encoded by a nucleic acid comprising a sequence set forth in any one of SEQ ID NOs: 19 to 30.
44. An envelope-binding protein comprising a sequence set forth in any one of SEQ ID NOs: 7 to 18.
45. An envelope-binding protein comprising a sequence expressed from or encoded by a nucleic acid comprising a sequence set forth in any one of SEQ ID NOs: 19 to 30.
46. A nucleic acid encoding or expressing the envelope-binding protein of any one of paragraphs 1 to 42 or 44.
47. The envelope-binding protein of any one of paragraphs 1 to 45, for use in production of an enveloped virus in a cell culture, wherein the cell culture comprises a cell line cultured in a cell culture medium.
48. The envelope-binding protein of any one of paragraphs 1 to 45, for use in purification of an enveloped virus from a cell culture.
49. The envelope-binding protein of any one of paragraphs 1 to 45, wherein the purification tag is not a glutathione S transferase (GST) tag.
50. A method of producing an enveloped virus in a cell culture, the method comprising culturing a cell line in a cell culture medium comprising a protein that specifically binds vesicular stomatitis virus G (VSV-G) pseudotyped virus.
51. The method of paragraph 50, wherein the protein comprises one or more low-density lipoprotein receptor (LDLR) domains.
52. The method of paragraphs 50 or 51, wherein the protein is the envelope-binding protein of any one of claims 1 to 45.
53. The method of any one of paragraphs 50 to 52, wherein the cell line is a stable producer cell line.
54. The method of any one of paragraphs 50 to 53, wherein the cell line expresses the protein.
55. The method of any one of paragraphs 50 to 54, wherein the protein is added to the cell culture medium.
56. The method of paragraph 55, wherein the protein is added to the cell culture medium at a concentration of at least 0.001 μg/mL.
57. The method of paragraphs 55 or 56, wherein the protein is added to the cell culture medium on each day of the cell culture.
58. The method of any one of paragraphs 50 to 57, wherein the method results in an increase in viral infectious titer yield by at least 1-fold compared to culturing in a cell culture medium in the absence of the protein.
59. A method of purifying an enveloped virus from a cell culture, the method comprising (i) culturing a cell line in a cell culture medium comprising a protein that specifically binds vesicular stomatitis virus G (VSV-G) pseudotyped virus and (ii) isolating the enveloped virus from the cell culture, wherein the protein is bound to the enveloped virus.
60. The method of paragraph 59, wherein the protein comprises one or more low-density lipoprotein receptor (LDLR) domains.
61. The method of paragraphs 59 or 60, wherein the protein is the envelope-binding protein of any one of claims 1 to 45.
62. The method of any one of paragraphs 59 to 61, wherein the method comprises affinity capture chromatography.
63. The method of paragraph 62, wherein the affinity capture chromatography is protein A chromatography.
64. A method of purifying an enveloped virus from a cell culture, the method comprising (i) culturing a cell line in a cell culture medium, (ii) harvesting the cell culture medium comprising the enveloped virus, (iii) loading the harvested cell culture medium comprising the enveloped virus onto an affinity chromatography resin comprising an envelope-binding protein of any one of paragraphs 1 to 45 immobilized to a matrix of the affinity chromatography resin and (iv) collecting the enveloped virus.
65. The method of any one of paragraphs 62 to 64, wherein the enveloped virus is loaded onto an affinity capture chromatography column in a loading buffer comprising calcium.
66. The method of paragraph 65, wherein the calcium is at a concentration of at least 0.01 mM.
67. The method of any one of paragraphs 62 to 66, wherein the affinity capture chromatography column is eluted with a buffer comprising less than 0.01 mM calcium.
68. The method of any one of paragraphs 59 to 67, wherein purifying the enveloped virus additionally comprises one or more steps selected from the group consisting of clarification filtration, anion exchange chromatography, concentration and diafiltration.
69. The method of paragraph 68, additionally comprising formulating the purified enveloped virus into a pharmaceutical formulation or into a solution suitable for infecting a cell.
70. The method of any one of paragraphs 50 to 69, wherein the enveloped virus is a retrovirus.
71. The method of paragraph 70, wherein the retrovirus is a lentivirus.
72. The method of paragraphs 70 or 71, wherein the enveloped virus is a VSV-G-pseudotyped virus.

SEQUENCES OF THE DISCLOSURE

| ID | Description | Sequence |
|---|---|---|
| SEQ ID | Amino acid sequence | MGPWGWKLRWTVALLLAAAGTAVGDRCERNEFQCQDG |
| NO: 1 | of Human low- | KCISYKWVCDGSAECQDGSDESQETCLSVTCKSGDFSCG |
| | density lipoprotein | GRVNRCIPQFWRCDGQVDCDNGSDEQGCPPKTCSQDEFR |
| | receptor (LDLR) | CHDGKCISRQFVCDSDRDCLDGSDEASCPVLTCGPASFQC |
| | | NSSTCIPQLWACDNDPDCEDGSDEWPQRCRGLYVFQGDS |
| | | SPCSAFEFHCLSGECIHSSWRCDGGPDCKDKSDEENCAVA |
| | | TCRPDEFQCSDGNCIHGSRQCDREYDCKDMSDEVGCVNV |
| | | TLCEGPNKFKCHSGECITLDKVCNMARDCRDWSDEPIKE |
| | | CGTNECLDNNGGCSHVCNDLKIGYECLCPDGFQLVAQRR |
| | | CEDIDECQDPDTCSQLCVNLEGGYKCQCEEGFQLDPHTK |
| | | ACKAVGSIAYLFFTNRHEVRKMTLDRSEYTSLIPNLRNVV |
| | | ALDTEVASNRIYWSDLSQRMICSTOLDRAHGVSSYDTVIS |
| | | RDIQAPDGLAVDWIHSNIYWTDSVLGTVSVADTKGVKRK |
| | | TLFRENGSKPRAIVVDPVHGFMYWTDWGTPAKIKKGGLN |
| | | GVDIYSLVTENIQWPNGITLDLLSGRLYWVDSKLHSISSID |
| | | VNGGNRKTILEDEKRLAHPFSLAVFEDKVFWTDIINEAIFS |
| | | ANRLTGSDVNLLAENLLSPEDMVLFHNLTQPRGVNWCER |
| | | TTLSNGGCQYLCLPAPQINPHSPKFTCACPDGMLLARDM |
| | | RSCLTEAEAAVATQETSTVRLKVSSTAVRTQHTTTRPVPD |
| | | TSRLPGATPGLTTVEIVTMSHQALGDVAGRGNEKKPSSVR |
| | | ALSIVLPIVLLVFLCLGVFLLWKNWRLKNINSINFDNPVYQ |
| | | KTTEDEVHICHNQDGYSYPSRQMVSLEDDVA |
| SEQ ID | Amino acid sequence | SVTCKSGDFSCGGRVNRCIPQFWRCDGQVDCDNGSDEQG |
| NO: 2 | of Human LDLR | CP |
| | complement-type | |
| | repeat 2 | |
| SEQ ID | Amino acid sequence | PKTCSQDEFRCHDGKCISRQFVCDSDRDCLDGSDEASCP |
| NO: 3 | of Human LDLR | |
| | complement-type | |
| | repeat 3 | |
| SEQ ID | Amino acid sequence | EPKSCDKTHTCPPCPAPELLGGPSVFLFPPKPKDTLMISRT |
| NO: 4 | of Human IgG1 Fc | PEVTCVVVDVSHEDPEVKFNWYVDGVEVHNAKTKPREE |
| | | QYNSTYRVVSVLTVLHQDWLNGKEYKCKVSNKALPAPIE |
| | | KTISKAKGQPREPQVYTLPPSRDELTKNQVSLTCLVKGFY |
| | | PSDIAVEWESNGQPENNYKTTPPVLDSDGSFFLYSKLTVD |
| | | KSRWQQGNVFSCSVMHEALHNHYTQKSLSLSPGK |
| SEQ ID | Ceruloplasmin (Cp) | MKILILGIFLFLCSTPAWA |
| NO: 5 | Signal peptide | |
| SEQ ID | HuIgG1Fc Signal | MGWSCIILFLVATATGVHS |
| NO: 6 | peptide | |
| SEQ ID | Amino acid sequence | MKILILGIFLFLCSTPAWASVTCKSGDFSCGGRVNRCIPQ |
| NO: 7 | of construct | FWRCDGQVDCDNGSDEQGCPGSEPKSCDKTHTCPPCPAPE |
| | HuLDLR(65-105)- | LLGGPSVFLFPPKPKDTLMISRTPEVTCVVVDVSHEDPEV |
| | GS-HuIgG1Fc | KFNWYVDGVEVHNAKTKPREEQYNSTYRVVSVLTVLHQ |
| | (including signal | DWLNGKEYKCKVSNKALPAPIEKTISKAKGQPREPQVYT |
| | peptide) | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| SEQ ID | Amino acid sequence | MKILILGIFLFLCSTPAWASVTCKSGDFSCGGRVNRCIPQF |
| NO: 8 | of construct | WRCDGQVDCDNGSDEQGCPPKTCSQDEFRCHDGKCISRQ |
| | HuLDLR(65-144)- | FVCDSDRDCLDGSDEASCPGSEPKSCDKTHTCPPCPAPEL |
| | GS-HuIgG1Fc-1 | LGGPSVFLFPPKPKDTLMISRTPEVTCVVVDVSHEDPEVK |
| | (including signal | FNWYVDGVEVHNAKTKPREEQYNSTYRVVSVLTVLHQD |
| | peptide) | WLNGKEYKCKVSNKALPAPIEKTISKAKGQPREPQVYTLP |
| | | PSRDELTKNQVSLTCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| SEQ ID | Amino acid sequence | MGWSCIILFLVATATGVHSEPKSCDKTHTCPPCPAPELLG |
| NO: 9 | of | GPSVFLFPPKPKDTLMISRTPEVTCVVVDVSHEDPEVKEN |
| | construct | WYVDGVEVHNAKTKPREEQYNSTYRVVSVLTVLHQDW |
| | HuIgG1Fc-GS- | LNGKEYKCKVSNKALPAPIEKTISKAKGQPREPQVYTLPP |
| | HuLDLR(65-105) | SRDELTKNQVSLTCLVKGFYPSDIAVEWESNGQPENNYK |
| | (including signal | TTPPVLDSDGSFFLYSKLTVDKSRWQQGNVFSCSVMHEA |
| | peptide) | LHNHYTQKSLSLSPGKGSSVTCKSGDFSCGGRVNRCIPQF |
| | | WRCDGQVDCDNGSDEQGCP |
| SEQ ID | Amino acid sequence | MGWSCIILFLVATATGVHSEPKSCDKTHTCPPCPAPELLG |
| NO: 10 | of construct | GPSVFLFPPKPKDTLMISRTPEVTCVVVDVSHEDPEVKFN |
| | HuIgG1Fc-GS- | WYVDGVEVHNAKTKPREEQYNSTYRVVSVLTVLHQDW |
| | HuLDLR(65-144) | LNGKEYKCKVSNKALPAPIEKTISKAKGQPREPQVYTLPP |
| | (including signal | SRDELTKNQVSLTCLVKGFYPSDIAVEWESNGQPENNYK |
| | peptide) | TTPPVLDSDGSFFLYSKLTVDKSRWQQGNVFSCSVMHEA |
| | | LHNHYTQKSLSLSPGKGSSVTCKSGDFSCGGRVNRCIPQF |
| | | WRCDGQVDCDNGSDEQGCPPKTCSQDEFRCHDGKCISRQ |
| | | FVCDSDRDCLDGSDEASCP |
| SEQ ID | Amino acid sequence | MKILILGIFLFLCSTPAWASVTCKSGDFSCGGRVNRCIPQF |
| NO: 11 | of construct | WRCDGQVDCDNGSDEQGCPGSGGSSVTCKSGDFSCGGR |
| | HuLDLR[(65- | VNRCIPQFWRCDGQVDCDNGSDEQGCPGGSSGSVTCKSG |
| | 105)x3]-GS- | DFSCGGRVNRCIPQFWRCDGQVDCDNGSDEQGCPGSEPK |
| | HuIgG1Fc (including | SCDKTHTCPPCPAPELLGGPSVFLFPPKPKDTLMISRTPEV |
| | signal peptide) | TCVVVDVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPSRDELTKNQVSLTCLVKGFYPSDI |
| | | AVEWESNGQPENNYKTTPPVLDSDGSFFLYSKLTVDKSR |
| | | WQQGNVFSCSVMHEALHNHYTQKSLSLSPGK |
| SEQ ID | Amino acid sequence | MKILILGIFLFLCSTPAWASVTCKSGDFSCGGRVNRCIPQF |
| NO: 12 | of construct | WRCDGQVDCDNGSDEQGCPGSGGSSVTCKSGDFSCGGR |
| | HuLDLR[(65- | VNRCIPQFWRCDGQVDCDNGSDEQGCPGGSSGSVTCKSG |
| | 105)x5]-GS- | DESCGGRVNRCIPQFWRCDGQVDCDNGSDEQGCPGGGG |
| | HuIgG1Fc (including | SSVTCKSGDFSCGGRVNRCIPQFWRCDGQVDCDNGSDEQ |
| | signal peptide) | GCPGSGSGSVTCKSGDFSCGGRVNRCIPQFWRCDGQVDC |
| | | DNGSDEQGCPGSEPKSCDKTHTCPPCPAPELLGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKC |
| | | KVSNKALPAPIEKTISKAKGQPREPQVYTLPPSRDELTKNQ |
| | | VSLTCLVKGFYPSDIAVEWESNGQPENNYKTTPPVLDSDG |
| | | SFFLYSKLTVDKSRWQQGNVFSCSVMHEALHNHYTQKSL |
| | | SLSPGK |
| SEQ ID | Amino acid sequence | MKILILGIFLFLCSTPAWAPKTCSQDEFRCHDGKCISRQFV |
| NO: 13 | of construct | CDSDRDCLDGSDEASCPGSGGSPKTCSQDEFRCHDGKCIS |
| | HuLDLR[(106- | RQFVCDSDRDCLDGSDEASCPGGSSGPKTCSQDEFRCHD |
| | 144)x3]-GS- | GKCISRQFVCDSDRDCLDGSDEASCPGSEPKSCDKTHTCP |
| | HuIgG1Fc (including | PCPAPELLGGPSVFLFPPKPKDTLMISRTPEVTCVVVDVSH |
| | signal peptide) | EDPEVKFNWYVDGVEVHNAKTKPREEQYNSTYRVVSVL |
| | | TVLHQDWLNGKEYKCKVSNKALPAPIEKTISKAKGQPRE |
| | | PQVYTLPPSRDELTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| SEQ ID | Amino acid sequence | MGWSCIILFLVATATGVHSEPKSCDKTHTCPPCPAPELLG |
| NO: 14 | of construct | GPSVFLFPPKPKDTLMISRTPEVTCVVVDVSHEDPEVKEN |
| | HuIgG1Fc-GS- | WYVDGVEVHNAKTKPREEQYNSTYRVVSVLTVLHQDW |
| | HuLDLR[(65- | LNGKEYKCKVSNKALPAPIEKTISKAKGQPREPQVYTLPP |
| | 105)x3] (including | SRDELTKNQVSLTCLVKGFYPSDIAVEWESNGQPENNYK |
| | signal peptide) | TTPPVLDSDGSFFLYSKLTVDKSRWQQGNVFSCSVMHEA |
| | | LHNHYTQKSLSLSPGKGSSVTCKSGDFSCGGRVNRCIPQF |
| | | WRCDGQVDCDNGSDEQGCPGSGGSSVTCKSGDFSCGGR |
| | | VNRCIPQFWRCDGQVDCDNGSDEQGCPGGSSGSVTCKSG |
| | | DESCGGRVNRCIPQFWRCDGQVDCDNGSDEQGCP |
| SEQ ID | Amino acid sequence | MGWSCIILFLVATATGVHSEPKSCDKTHTCPPCPAPELLG |
| NO: 15 | of construct | GPSVFLFPPKPKDTLMISRTPEVTCVVVDVSHEDPEVKEN |
| | HuIgG1Fc-GS- | WYVDGVEVHNAKTKPREEQYNSTYRVVSVLTVLHQDW |
| | HuLDLR[(65- | LNGKEYKCKVSNKALPAPIEKTISKAKGQPREPQVYTLPP |
| | 105)x5] (including | SRDELTKNQVSLTCLVKGFYPSDIAVEWESNGQPENNYK |
| | signal peptide) | TTPPVLDSDGSFFLYSKLTVDKSRWQQGNVFSCSVMHEA |
| | | LHNHYTQKSLSLSPGKGSSVTCKSGDFSCGGRVNRCIPQF |
| | | WRCDGQVDCDNGSDEQGCPGSGGSSVTCKSGDFSCGGR |
| | | VNRCIPQFWRCDGQVDCDNGSDEQGCPGGSSGSVTCKSG |
| | | DFSCGGRVNRCIPQFWRCDGQVDCDNGSDEQGCPGGGG |
| | | SSVTCKSGDFSCGGRVNRCIPQFWRCDGQVDCDNGSDEQ |
| | | GCPGSGSGSVTCKSGDFSCGGRVNRCIPQFWRCDGQVDC |
| | | DNGSDEQGCP |
| SEQ ID | Amino acid sequence | MGWSCIILFLVATATGVHSEPKSCDKTHTCPPCPAPELLG |
| NO: 16 | of construct | GPSVFLFPPKPKDTLMISRTPEVTCVVVDVSHEDPEVKEN |
| | HuIgG1Fc-GS- | WYVDGVEVHNAKTKPREEQYNSTYRVVSVLTVLHQDW |
| | HuLDLR[(106- | LNGKEYKCKVSNKALPAPIEKTISKAKGQPREPQVYTLPP |
| | 144)x3] (including | SRDELTKNQVSLTCLVKGFYPSDIAVEWESNGQPENNYK |
| | signal peptide) | TTPPVLDSDGSFFLYSKLTVDKSRWQQGNVFSCSVMHEA |
| | | LHNHYTQKSLSLSPGKGSPKTCSQDEFRCHDGKCISRQFV |
| | | CDSDRDCLDGSDEASCPGSGGSPKTCSQDEFRCHDGKCIS |
| | | RQFVCDSDRDCLDGSDEASCPGGSSGPKTCSQDEFRCHD |
| | | GKCISRQFVCDSDRDCLDGSDEASCP |
| SEQ ID | Amino acid sequence | MKILILGIFLFLCSTPAWAPKTCSQDEFRCHDGKCISRQFV |
| NO: 17 | of construct | CDSDRDCLDGSDEASCPGSEPKSCDKTHTCPPCPAPELLG |
| | huLDLR(65-105)- | GPSVFLFPPKPKDTLMISRTPEVTCVVVDVSHEDPEVKEN |
| | GS-huIgG1Fc-GS- | WYVDGVEVHNAKTKPREEQYNSTYRVVSVLTVLHQDW |
| | huLDLR(65-105) | LNGKEYKCKVSNKALPAPIEKTISKAKGQPREPQVYTLPP |
| | (including signal | SRDELTKNQVSLTCLVKGFYPSDIAVEWESNGQPENNYK |
| | peptide) | TTPPVLDSDGSFFLYSKLTVDKSRWQQGNVFSCSVMHEA |
| | | LHNHYTQKSLSLSPGKGSPKTCSQDEFRCHDGKCISRQFV |
| | | CDSDRDCLDGSDEASCP |
| SEQ ID | Amino acid sequence | MKILILGIFLFLCSTPAWAPKTCSQDEFRCHDGKCISRQFV |
| NO: 18 | of construct | CDSDRDCLDGSDEASCPGSEPKSCDKTHTCPPCPAPELLG |
| | huLDLR(106-144)- | GPSVFLFPPKPKDTLMISRTPEVTCVVVDVSHEDPEVKFN |
| | GS-huIgG1Fc-GS- | WYVDGVEVHNAKTKPREEQYNSTYRVVSVLTVLHQDW |
| | huLDLR(106-144) | LNGKEYKCKVSNKALPAPIEKTISKAKGQPREPQVYTLPP |
| | (including signal | SRDELTKNQVSLTCLVKGFYPSDIAVEWESNGQPENNYK |
| | peptide) | TTPPVLDSDGSFFLYSKLTVDKSRWQQGNVFSCSVMHEA |
| | | LHNHYTQKSLSLSPGKGSPKTCSQDEFRCHDGKCISRQFV |
| | | CDSDRDCLDGSDEASCP |
| SEQ ID | Nucleic acid sequence | gctagcgccaccatgaagatcctgatcctgggcatcttcctgtttctgtgcagcacccctg |
| NO: 19 | of construct | cctgggcaagcgtgacctgcaagagcggcgacttcagctgtggcggcagagtgaacagatg |
| | HuLDLR(65-105)- | catcccccagttttggagatgcgacggccaggtggactgcgacaatggctctgacgagca |
| | GS-HuIgG1Fc | gggctgccccggatccgagcccaaatcttgtgacaaaactcacacatgcccaccgtgccc |
| | | agcacctgaactcctggggggaccgtcagtcttcctcttccccccaaaacccaaggacacc |
| | | ctcatgatctcccggacccctgaggtcacatgcgtggtggtggacgtgagccacgaagac |
| | | cctgaggtcaagttcaactggtacgtggacggcgtggaggtgcataatgccaagacaaag |
| | | ccgcgggaggagcagtacaacagcacgtaccgtgtggtcagcgtcctcaccgtcctgcac |
| | | caggactggctgaatggcaaggagtacaagtgcaaggtctccaacaaagccctcccagcc |
| | | cccatcgagaaaaccatctccaaagccaaagggcagccccgagaaccacaggtgtacac |
| | | cctgcccccatcccgggatgagctgaccaagaaccaggtcagcctgacctgcctggtcaa |
| | | aggcttctatcccagcgacatcgccgtggagtgggagagcaatgggcagccggagaacaa |
| | | ctacaagaccacgcctcccgtgctggactccgacggctccttcttcctctacagcaagctc |
| | | accgtggacaagagcaggtggcagcaggggaacgtcttctcatgctccgtgatgcatgag |
| | | gctctgcacaaccactacacgcagaagagcctctccctgtctccgggtaaatgataactcga |
| | | g |
| SEQ ID | Nucleic acid sequence | gctagcgccaccatgaagatcctgatcctgggcatcttcctgtttctgtgcagcacccctg |
| NO: 20 | of construct | cctgggcaagcgtgacctgcaagagcggcgacttcagctgtggcggcagagtgaacagatg |
| | HuLDLR(65-144)- | catcccccagttttggagatgcgacggccaggtggactgcgacaatggctctgacgagca |
| | GS-HuIgG1Fc-1 | gggctgcccccctaagacctgcagccaggacgagttcagatgtcacgatggcaagtgtat |
| | | ctcccggcagttcgtgtgcgacagcgacagagactgcctggacggctctgatgaggcctc |
| | | ctgtcctggatccgagcccaaatcttgtgacaaaactcacacatgcccaccgtgcccagca |
| | | cctgaactcctggggggaccgtcagtcttcctcttccccccaaaacccaaggacaccctcat |
| | | gatctcccggacccctgaggtcacatgcgtggtggtggacgtgagccacgaagaccctga |
| | | ggtcaagttcaactggtacgtggacggcgtggaggtgcataatgccaagacaaagccgcg |
| | | ggaggagcagtacaacagcacgtaccgtgtggtcagcgtcctcaccgtcctgcaccagga |
| | | ctggctgaatggcaaggagtacaagtgcaaggtctccaacaaagccctcccagcccccat |
| | | cgagaaaaccatctccaaagccaaagggcagccccgagaaccacaggtgtacaccctgc |
| | | ccccatcccgggatgagctgaccaagaaccaggtcagcctgacctgcctggtcaaaggct |
| | | tctatcccagcgacatcgccgtggagtgggagagcaatgggcagccggagaacaactac |
| | | aagaccacgcctcccgtgctggactccgacggctccttcttcctctacagcaagctcaccgt |
| | | ggacaagagcaggtggcagcaggggaacgtcttctcatgctccgtgatgcatgaggctct |
| | | gcacaaccactacacgcagaagagcctctccctgtctccgggtaaatgataactcgag |
| SEQ ID | Nucleic acid sequence | gctagcgccaccatgggatggagctgtatcatcctcttcttggtagcaacagctacaggtaa |
| NO: 21 | of construct | ggggttaacagtagcaggcttgaggtctggacatatatatgggtgacaatgacatccactttg |
| | HuIgG1Fc-GS- | cctttctctccacaggcgtgcactctgagcccaaatcttgtgacaaaactcacacatgcccac |
| | HuLDLR(65-105) | cgtgcccagcacctgaactcctggggggaccgtcagtcttcctcttccccccaaaacccaa |
| | (intronic sequence | ggacaccctcatgatctcccggacccctgaggtcacatgcgtggtggtggacgtgagcca |
| | underlined) | cgaagaccctgaggtcaagttcaactggtacgtggacggcgtggaggtgcataatgccaa |
| | | gacaaagccgcgggaggagcagtacaacagcacgtaccgtgtggtcagcgtcctcaccg |
| | | tcctgcaccaggactggctgaatggcaaggagtacaagtgcaaggtctccaacaaagccc |
| | | tcccagcccccatcgagaaaaccatctccaaagccaaagggcagccccgagaaccacag |
| | | gtgtacaccctgcccccatcccgggatgagctgaccaagaaccaggtcagcctgacctgc |
| | | ctggtcaaaggcttctatcccagcgacatcgccgtggagtgggagagcaatgggcagccg |
| | | gagaacaactacaagaccacgcctcccgtgctggactccgacggctccttcttcctctacag |
| | | caagctcaccgtggacaagagcaggtggcagcaggggaacgtcttctcatgctccgtgat |
| | | gcatgaggctctgcacaaccactacacgcagaagagcctctccctgtctccgggtaaagg |
| | | atccagcgtgacctgcaagagcggcgacttcagctgtggcggcagagtgaacagatgcat |
| | | cccccagttttggagatgcgacggccaggtggactgcgacaatggctctgacgagcaggg |
| | | ctgcccctgataactcgag |
| SEQ ID | Nucleic acid sequence | gctagcgccaccatgggatggagctgtatcatcctcttcttggtagcaacagctacaggtaa |
| NO: 22 | of construct | ggggttaacagtagcaggcttgaggtctggacatatatatgggtgacaatgacatccactttg |
| | HuIgG1Fc-GS- | cctttctctccacaggcgtgcactctgagcccaaatcttgtgacaaaactcacacatgcccac |
| | HuLDLR(65-144) | cgtgcccagcacctgaactcctggggggaccgtcagtcttcctcttccccccaaaacccaa |
| | (intronic sequence | ggacaccctcatgatctcccggacccctgaggtcacatgcgtggtggtggacgtgagcca |
| | underlined) | cgaagaccctgaggtcaagttcaactggtacgtggacggcgtggaggtgcataatgccaa |
| | | gacaaagccgcgggaggagcagtacaacagcacgtaccgtgtggtcagcgtcctcaccg |
| | | tcctgcaccaggactggctgaatggcaaggagtacaagtgcaaggtctccaacaaagccc |
| | | tcccagcccccatcgagaaaaccatctccaaagccaaagggcagccccgagaaccacag |
| | | gtgtacaccctgcccccatcccgggatgagctgaccaagaaccaggtcagcctgacctgc |
| | | ctggtcaaaggcttctatcccagcgacatcgccgtggagtgggagagcaatgggcagccg |
| | | gagaacaactacaagaccacgcctcccgtgctggactccgacggctccttcttcctctacag |
| | | caagctcaccgtggacaagagcaggtggcagcaggggaacgtcttctcatgctccgtgat |
| | | gcatgaggctctgcacaaccactacacgcagaagagcctctccctgtctccgggtaaagg |
| | | atccagcgtgacctgcaagagcggcgacttcagctgtggcggcagagtgaacagatgcat |
| | | cccccagttttggagatgcgacggccaggtggactgcgacaatggctctgacgagcaggg |
| | | ctgcccccctaagacctgcagccaggacgagttcagatgtcacgatggcaagtgtatctcc |
| | | cggcagttcgtgtgcgacagcgacagagactgcctggacggctctgatgaggcctcctgt |
| | | ccttgataactcgag |
| SEQ ID | Nucleic acid | gctagcgccaccatgaagatcctgatcctgggcatcttcctgtttctgtgcagcacaccagc |
| NO: 23 | sequence | ctgggccagcgtgacatgtaaaagcggcgatttcagctgcggcggcagagtgaacagatg |
| | of construct | catccctcagttttggagatgcgacggccaggtggactgcgacaatggctctgatgagcag |
| | HuLDLR[(65- | ggctgtcctggcagcggaggatcttctgtgacatgcaagtccggcgacttctcctgtggcg |
| | 105)x3]-GS- | gaagggtcaacagatgtattccgcaattttggcgctgtgatggacaggtcgactgtgacaac |
| | HuIgG1Fc | ggaagcgacgaacagggatgtccaggcggcagttctggaagcgtgacctgcaagagcg |
| | | gagacttttcctgcggaggccgggtcaaccgttgcattccacaattttggcgttgcgacgga |
| | | caagttgattgcgataacggttccgacgagcaaggctgcccaggatccgagcccaaatctt |
| | | gtgacaaaactcacacatgcccaccgtgcccagcacctgaactcctggggggaccgtcag |
| | | tcttcctcttccccccaaaacccaaggacaccctcatgatctcccggacccctgaggtcaca |
| | | tgcgtggtggtggacgtgagccacgaagaccctgaggtcaagttcaactggtacgtggac |
| | | ggcgtggaggtgcataatgccaagacaaagccgcgggaggagcagtacaacagcacgt |
| | | accgtgtggtcagcgtcctcaccgtcctgcaccaggactggctgaatggcaaggagtaca |
| | | agtgcaaggtctccaacaaagccctcccagcccccatcgagaaaaccatctccaaagcca |
| | | aagggcagccccgagaaccacaggtgtacaccctgcccccatcccgggatgagctgacc |
| | | aagaaccaggtcagcctgacctgcctggtcaaaggcttctatcccagcgacatcgccgtgg |
| | | agtgggagagcaatgggcagccggagaacaactacaagaccacgcctcccgtgctgga |
| | | ctccgacggctccttcttcctctacagcaagctcaccgtggacaagagcaggtggcagcag |
| | | gggaacgtcttctcatgctccgtgatgcatgaggctctgcacaaccactacacgcagaaga |
| | | gcctctccctgtctccgggtaaatgataactcgag |
| SEQ ID | Nucleic acid | gctagcgccaccatgaagatcctgatcctgggcatcttcctgtttctgtgcagcacaccagc |
| NO: 24 | sequence | ctgggccagcgtgacatgtaaaagcggcgatttcagctgcggcggcagagtgaacagatg |
| | of construct | catccctcagttttggagatgcgacggccaggtggactgcgacaatggctctgatgagcag |
| | HuLDLR[(65- | ggctgtcctggcagcggaggatcttctgtgacatgcaagtccggcgacttctcctgtggcg |
| | 105)x5]-GS- | gaagggtcaacagatgtattccgcaattttggcgctgtgatggacaggtcgactgtgacaac |
| | HuIgG1Fc | ggaagcgacgaacagggatgtccaggcggcagttctggaagcgtgacctgcaagagcg |
| | | gagacttttcctgcggaggccgggtcaaccgttgcattccacaattttggcgttgcgacgga |
| | | caagttgattgcgataacggttccgacgagcaaggctgcccaggcggaggtggaagttcc |
| | | gtgacttgtaaatctggcgatttttcttgcggcggacgcgtgaaccggtgtatcccacaatt |
| | | ttggagatgtgatgggcaagtcgattgtgataatggcagtgacgagcaggggtgccctggat |
| | | ctggttctggatctgtcacctgtaaatccggggactttagttgcggtggaagagtgaatcggt |
| | | gcattccacagttttggcgctgtgacggccaagtggactgtgataacggatcagatgaaca |
| | | gggctgccccggatccgagcccaaatcttgtgacaaaactcacacatgcccaccgtgccc |
| | | agcacctgaactcctggggggaccgtcagtcttcctcttccccccaaaacccaaggacacc |
| | | ctcatgatctcccggacccctgaggtcacatgcgtggtggtggacgtgagccacgaagac |
| | | cctgaggtcaagttcaactggtacgtggacggcgtggaggtgcataatgccaagacaaag |
| | | ccgcgggaggagcagtacaacagcacgtaccgtgtggtcagcgtcctcaccgtcctgcac |
| | | caggactggctgaatggcaaggagtacaagtgcaaggtctccaacaaagccctcccagcc |
| | | cccatcgagaaaaccatctccaaagccaaagggcagccccgagaaccacaggtgtacac |
| | | cctgcccccatcccgggatgagctgaccaagaaccaggtcagcctgacctgcctggtcaa |
| | | aggcttctatcccagcgacatcgccgtggagtgggagagcaatgggcagccggagaaca |
| | | actacaagaccacgcctcccgtgctggactccgacggctccttcttcctctacagcaagctc |
| | | accgtggacaagagcaggtggcagcaggggaacgtcttctcatgctccgtgatgcatgag |
| | | gctctgcacaaccactacacgcagaagagcctctccctgtctccgggtaaatgataactcga |
| | | g |
| SEQ ID | Nucleic acid | gctagcgccaccatgaagatcctgatcctgggcatcttcctgtttctgtgcagcacccctg |
| NO: 25 | sequence | cctgggctcctaagacatgtagccaggacgagttcagatgccacgacggcaagtgcatcag |
| | of construct | ccggcagttcgtgtgcgacagcgacagagattgtctggacggcagcgacgaggcctcttgt |
| | HuLDLR[(106- | cctggatctggcggaagcccaaagacctgctctcaggatgagttccggtgtcacgatggaa |
| | 144)x3]-GS- | agtgtatctcccgccagtttgtgtgtgactccgacagggactgcctggatggctccgatgaa |
| | HuIgG1Fc | gcttcttgtccaggcggcagcagcggccctaaaacatgttcccaggatgaatttcggtgcca |
| | | tgacgggaaatgcatctccagacagttcgtctgtgatagcgaccgcgactgcctcgacgga |
| | | tctgatgaggcaagttgtcctggatccgagcccaaatcttgtgacaaaactcacacatgccc |
| | | accgtgcccagcacctgaactcctggggggaccgtcagtcttcctcttccccccaaaaccc |
| | | aaggacaccctcatgatctcccggacccctgaggtcacatgcgtggtggtggacgtgagc |
| | | cacgaagaccctgaggtcaagttcaactggtacgtggacggcgtggaggtgcataatgcc |
| | | aagacaaagccgcgggaggagcagtacaacagcacgtaccgtgtggtcagcgtcctcac |
| | | cgtcctgcaccaggactggctgaatggcaaggagtacaagtgcaaggtctccaacaaagc |
| | | cctcccagcccccatcgagaaaaccatctccaaagccaaagggcagccccgagaaccac |
| | | aggtgtacaccctgcccccatcccgggatgagctgaccaagaaccaggtcagcctgacct |
| | | gcctggtcaaaggcttctatcccagcgacatcgccgtggagtgggagagcaatgggcagc |
| | | cggagaacaactacaagaccacgcctcccgtgctggactccgacggctccttcttcctctac |
| | | agcaagctcaccgtggacaagagcaggtggcagcaggggaacgtcttctcatgctccgtg |
| | | atgcatgaggctctgcacaaccactacacgcagaagagcctctccctgtctccgggtaaat |
| | | gataactcgag |
| SEQ ID | Nucleic acid sequence | gctagcgccaccatgggatggagctgtatcatcctcttcttggtagcaacagctacaggta |
| NO: 26 | of construct | aggggttaacagtagcaggcttgaggtctggacatatatatgggtgacaatgacatccact |
| | HuIgG1Fc-GS- | ttgcctttctctccacaggcgtgcactctgagcccaaatcttgtgacaaaactcacacatg |
| | HuLDLR[(65- | cccaccgtgcccagcacctgaactcctggggggaccgtcagtcttcctcttccccccaaaa |
| | 105)x3] (intronic | cccaaggacaccctcatgatctcccggacccctgaggtcacatgcgtggtggtggacgtgag |
| | sequence underlined) | ccacgaagaccctgaggtcaagttcaactggtacgtggacggcgtggaggtgcataatgcca |
| | | agacaaagccgcgggaggagcagtacaacagcacgtaccgtgtggtcagcgtcctcaccg |
| | | tcctgcaccaggactggctgaatggcaaggagtacaagtgcaaggtctccaacaaagccc |
| | | tcccagcccccatcgagaaaaccatctccaaagccaaagggcagccccgagaaccacag |
| | | gtgtacaccctgcccccatcccgggatgagctgaccaagaaccaggtcagcctgacctgc |
| | | ctggtcaaaggcttctatcccagcgacatcgccgtggagtgggagagcaatgggcagccg |
| | | gagaacaactacaagaccacgcctcccgtgctggactccgacggctccttcttcctctacag |
| | | caagctcaccgtggacaagagcaggtggcagcaggggaacgtcttctcatgctccgtgat |
| | | gcatgaggctctgcacaaccactacacgcagaagagcctctccctgtctccgggtaaagg |
| | | atccagcgtgacatgtaaaagcggcgatttcagctgcggcggcagagtgaacagatgcat |
| | | ccctcagttttggagatgcgacggccaggtggactgcgacaatggctctgatgagcaggg |
| | | ctgtcctggcagcggaggatcttctgtgacatgcaagtccggcgacttctcctgtggcggaa |
| | | gggtcaacagatgtattccgcaattttggcgctgtgatggacaggtcgactgtgacaacgga |
| | | agcgacgaacagggatgtccaggcggcagttctggaagcgtgacctgcaagagcggag |
| | | acttttcctgcggaggccgggtcaaccgttgcattccacaattttggcgttgcgacggacaa |
| | | gttgattgcgataacggttccgacgagcaaggctgcccatgataactcgag |
| SEQ ID | Nucleic acid sequence | gctagcgccaccatgggatggagctgtatcatcctcttcttggtagcaacagctacaggtaa |
| NO: 27 | of construct | ggggttaacagtagcaggcttgaggtctggacatatatatgggtgacaatgacatccactttg |
| | HuIgG1Fc-GS- | cctttctctccacaggcgtgcactctgagcccaaatcttgtgacaaaactcacacatgcccac |
| | HuLDLR[(65- | cgtgcccagcacctgaactcctggggggaccgtcagtcttcctcttccccccaaaacccaa |
| | 105)x5] (intronic | ggacaccctcatgatctcccggacccctgaggtcacatgcgtggtggtggacgtgagcca |
| | sequence underlined) | cgaagaccctgaggtcaagttcaactggtacgtggacggcgtggaggtgcataatgccaa |
| | | gacaaagccgcgggaggagcagtacaacagcacgtaccgtgtggtcagcgtcctcaccg |
| | | tcctgcaccaggactggctgaatggcaaggagtacaagtgcaaggtctccaacaaagccc |
| | | tcccagcccccatcgagaaaaccatctccaaagccaaagggcagccccgagaaccacag |
| | | gtgtacaccctgcccccatcccgggatgagctgaccaagaaccaggtcagcctgacctgc |
| | | ctggtcaaaggcttctatcccagcgacatcgccgtggagtgggagagcaatgggcagccg |
| | | gagaacaactacaagaccacgcctcccgtgctggactccgacggctccttcttcctctacag |
| | | caagctcaccgtggacaagagcaggtggcagcaggggaacgtcttctcatgctccgtgat |
| | | gcatgaggctctgcacaaccactacacgcagaagagcctctccctgtctccgggtaaagg |
| | | atccagcgtgacatgtaaaagcggcgatttcagctgcggcggcagagtgaacagatgcat |
| | | ccctcagttttggagatgcgacggccaggtggactgcgacaatggctctgatgagcaggg |
| | | ctgtcctggcagcggaggatcttctgtgacatgcaagtccggcgacttctcctgtggcggaa |
| | | gggtcaacagatgtattccgcaattttggcgctgtgatggacaggtcgactgtgacaacgga |
| | | agcgacgaacagggatgtccaggcggcagttctggaagcgtgacctgcaagagcggag |
| | | acttttcctgcggaggccgggtcaaccgttgcattccacaattttggcgttgcgacggacaa |
| | | gttgattgcgataacggttccgacgagcaaggctgcccaggcggaggtggaagttccgtgac |
| | | ttgtaaatctggcgatttttcttgcggcggacgcgtgaaccggtgtatcccacaattttgga |
| | | gatgtgatgggcaagtcgattgtgataatggcagtgacgagcaggggtgccctggatctggt |
| | | tctggatctgtcacctgtaaatccggggactttagttgcggtggaagagtgaatcggtgcatt |
| | | ccacagttttggcgctgtgacggccaagtggactgtgataacggatcagatgaacagggct |
| | | gcccctgataactcgag |
| SEQ ID | Nucleic acid sequence | gctagcgccaccatgggatggagctgtatcatcctcttcttggtagcaacagctacaggtaa |
| NO: 28 | of construct | ggggttaacagtagcaggcttgaggtctggacatatatatgggtgacaatgacatccactttg |
| | HuIgG1Fc-GS- | cctttctctccacaggcgtgcactctgagcccaaatcttgtgacaaaactcacacatgcccac |
| | HuLDLR[(106- | cgtgcccagcacctgaactcctggggggaccgtcagtcttcctcttccccccaaaacccaa |
| | 144)x3] (intronic | ggacaccctcatgatctcccggacccctgaggtcacatgcgtggtggtggacgtgagcca |
| | sequence underlined) | cgaagaccctgaggtcaagttcaactggtacgtggacggcgtggaggtgcataatgccaa |
| | | gacaaagccgcgggaggagcagtacaacagcacgtaccgtgtggtcagcgtcctcaccg |
| | | tcctgcaccaggactggctgaatggcaaggagtacaagtgcaaggtctccaacaaagccc |
| | | tcccagcccccatcgagaaaaccatctccaaagccaaagggcagccccgagaaccacag |
| | | gtgtacaccctgcccccatcccgggatgagctgaccaagaaccaggtcagcctgacctgc |
| | | ctggtcaaaggcttctatcccagcgacatcgccgtggagtgggagagcaatgggcagccg |
| | | gagaacaactacaagaccacgcctcccgtgctggactccgacggctccttcttcctctacag |
| | | caagctcaccgtggacaagagcaggtggcagcaggggaacgtcttctcatgctccgtgat |
| | | gcatgaggctctgcacaaccactacacgcagaagagcctctccctgtctccgggtaaagg |
| | | atcccctaagacatgtagccaggacgagttcagatgccacgacggcaagtgcatcagccg |
| | | gcagttcgtgtgcgacagcgacagagattgtctggacggcagcgacgaggcctcttgtcct |
| | | ggatctggcggaagcccaaagacctgctctcaggatgagttccggtgtcacgatggaaagt |
| | | gtatctcccgccagtttgtgtgtgactccgacagggactgcctggatggctccgatgaagctt |
| | | cttgtccaggcggcagcagcggccctaaaacatgttcccaggatgaatttcggtgccatga |
| | | cgggaaatgcatctccagacagttcgtctgtgatagcgaccgcgactgcctcgacggatct |
| | | gatgaggcaagttgtccttgataactcgag |
| SEQ ID | Nucleic acid sequence | gctagcgccaccatgaagatcctgatcctgggcatcttcctgtttctgtgcagcacccctgcc |
| NO: 29 | of construct | tgggctcctaaaacatgttcccaggatgaatttcggtgccatgacgggaaatgcatctccag |
| | huLDLR(65-105)- | acagttcgtctgtgatagcgaccgcgactgcctcgacggatctgatgaggcaagttgtcctg |
| | GS-huIgG1Fc-GS- | gatccgagcccaaatcttgtgacaaaactcacacatgcccaccgtgcccagcacctgaact |
| | huLDLR(65-105) | cctggggggaccgtcagtcttcctcttccccccaaaacccaaggacaccctcatgatctccc |
| | | ggacccctgaggtcacatgcgtggtggtggacgtgagccacgaagaccctgaggtcaagt |
| | | tcaactggtacgtggacggcgtggaggtgcataatgccaagacaaagccgcgggaggag |
| | | cagtacaacagcacgtaccgtgtggtcagcgtcctcaccgtcctgcaccaggactggctga |
| | | atggcaaggagtacaagtgcaaggtctccaacaaagccctcccagcccccatcgagaaaa |
| | | ccatctccaaagccaaagggcagccccgagaaccacaggtgtacaccctgcccccatcc |
| | | cgggatgagctgaccaagaaccaggtcagcctgacctgcctggtcaaaggcttctatccca |
| | | gcgacatcgccgtggagtgggagagcaatgggcagccggagaacaactacaagaccac |
| | | gcctcccgtgctggactccgacggctccttcttcctctacagcaagctcaccgtggacaaga |
| | | gcaggtggcagcaggggaacgtcttctcatgctccgtgatgcatgaggctctgcacaacca |
| | | ctacacgcagaagagcctctccctgtctccgggtaaaggatcccctaaaacatgttcccag |
| | | gatgaatttcggtgccatgacgggaaatgcatctccagacagttcgtctgtgatagcgaccg |
| | | cgactgcctcgacggatctgatgaggcaagttgtccttgataatgataactcgag |
| SEQ ID | Nucleic acid sequence | gctagcgccaccatgaagatcctgatcctgggcatcttcctgtttctgtgcagcacccctgcc |
| NO: 30 | of construct | tgggctcctaaaacatgttcccaggatgaatttcggtgccatgacgggaaatgcatctccag |
| | huLDLR(106-144)- | acagttcgtctgtgatagcgaccgcgactgcctcgacggatctgatgaggcaagttgtcctg |
| | GS-huIgG1Fc-GS- | gatccgagcccaaatcttgtgacaaaactcacacatgcccaccgtgcccagcacctgaact |
| | huLDLR(106-144) | cctggggggaccgtcagtcttcctcttccccccaaaacccaaggacaccctcatgatctccc |
| | | ggacccctgaggtcacatgcgtggtggtggacgtgagccacgaagaccctgaggtcaagt |
| | | tcaactggtacgtggacggcgtggaggtgcataatgccaagacaaagccgcgggaggag |
| | | cagtacaacagcacgtaccgtgtggtcagcgtcctcaccgtcctgcaccaggactggctga |
| | | atggcaaggagtacaagtgcaaggtctccaacaaagccctcccagcccccatcgagaaaa |
| | | ccatctccaaagccaaagggcagccccgagaaccacaggtgtacaccctgcccccatcc |
| | | cgggatgagctgaccaagaaccaggtcagcctgacctgcctggtcaaaggcttctatccca |
| | | gcgacatcgccgtggagtgggagagcaatgggcagccggagaacaactacaagaccac |
| | | gcctcccgtgctggactccgacggctccttcttcctctacagcaagctcaccgtggacaaga |
| | | gcaggtggcagcaggggaacgtcttctcatgctccgtgatgcatgaggctctgcacaacca |
| | | ctacacgcagaagagcctctccctgtctccgggtaaaggatcccctaaaacatgttcccag |
| | | gatgaatttcggtgccatgacgggaaatgcatctccagacagttcgtctgtgatagcgaccg |
| | | cgactgcctcgacggatctgatgaggcaagttgtccttgataatgataactcgag |
| SEQ ID | Amino acid sequence | DRCERNEFQCQDGKCISYKWVCDGSAECQDGSDESQETC |
| NO: 31 | of Human LDLR | LS |
| | complement-type | |
| | repeat 1 | |
| SEQ ID | Amino acid sequence | VTCKSGDFSCGGRVNRCIPQFWRCDGQVDCDNGSDEQG |
| NO: 32 | of Human I LDLR | CPP |
| | complement-type | |
| | repeat 2 | |
| SEQ ID | Amino acid sequence | KTCSQDEFRCHDGKCISRQFVCDSDRDCLDGSDEASCPV |
| NO: 33 | of Human LDLR | |
| | complement-type | |
| | repeat 3 | |
| SEQ ID | Amino acid sequence | LTCGPASFQCNSSTCIPQLWACDNDPDCEDGSDEWPQRC |
| NO: 34 | of Human LDLR | RG |
| | complement-type | |
| | repeat 4 | |
| SEQ ID | Amino acid sequence | SPCSAFEFHCLSGECIHSSWRCDGGPDCKDKSDEENCAV |
| NO: 35 | of Human LDLR | |
| | complement-type | |
| | repeat 5 | |
| SEQ ID | Amino acid sequence | ATCRPDEFQCSDGNCIHGSRQCDREYDCKDMSDEVGCVN |
| NO: 36 | of Human LDLR | |
| | complement-type | |
| | repeat 6 | |
| SEQ ID | Amino acid sequence | TLCEGPNKFKCHSGECITLDKVCNMARDCRDWSDEPIKE |
| NO: 37 | of Human LDLR | C |
| | complement-type | |
| | repeat 7 | |
| SEQ ID | Amino acid sequence | VGDRCERNEFQCQDGKCISYKWVCDGSAECQDGSDESQE |
| NO: 38 | of Human LDLR | TCL |
| | complement-type | |
| | repeat 1 | |
| SEQ ID | Amino acid sequence | VLTCGPASFQCNSSTCIPQLWACDNDPDCEDGSDEWPQR |
| NO: 39 | of Human LDLR | CR |
| | complement-type | |
| | repeat 4 | |
| SEQ ID | Amino acid sequence | DSSPCSAFEFHCLSGECIHSSWRCDGGPDCKDKSDEENCA |
| NO: 40 | of Human LDLR | |
| | complement-type | |
| | repeat 5 | |
| SEQ ID | Amino acid sequence | VATCRPDEFQCSDGNCIHGSRQCDREYDCKDMSDEVGCV |
| NO: 41 | of Human LDLR | |
| | complement-type | |
| | repeat 6 | |
| SEQ ID | Amino acid sequence | VTLCEGPNKFKCHSGECITLDKVCNMARDCRDWSDEPIK |
| NO: 42 | of Human LDLR | EC |
| | complement-type | |
| | repeat 7 | |

The present disclosure is described further in the following non-limiting examples.

### EXAMPLES

### Example 1: Production of Recombinant Envelope-Binding Proteins

Recombinant envelope-binding proteins were generated comprising CR2 and/or CR3 LDLR domains (as defined in Nikolic et al., Nat Commun 9, 1029 (2018), and a human IgG1 Fc as follows:
HuLDLR(65-105)-GS-HuIgG1Fc (SEQ TD NO: 7)
HuLDLR(65-144)-GS-HuIgG1Fc-1 (SEQ ID NO: 8)
HuIgG1Fc-GS-HuLDLR(65-105) (SEQ ID NO: 9)
HuIgG1Fc-GS-HuLDLR(65-144) (SEQ ID NO: 10)
HuLDLR[(65-105)x3]-GS-HuIgG1Fc (SEQ ID NO: 11)
HuLDLR[(65-105)x5]-GS-HuIgG1Fc (SEQ ID NO: 12)
HuLDLR[(106-144)x3]-GS-HuIgG1Fc (SEQ ID NO: 13)
HuIgG1Fc-GS-HuLDLR[(65-105)x3](SEQ ID NO: 14)
HuIgG1Fc-GS-HuLDLR[(65-105)x5](SEQ ID NO: 15)
HuIgG1Fc-GS-HuLDLR[(106-144)x3](SEQ ID NO: 16)
HuLDLR(65-105)-GS-HuIgG1Fc-GS-HuLDLR(65-105) (SEQ ID NO: 17)
HuLDLR(106-144)-GS-HuIgG1Fc-GS-HuLDLR(106-144) (SEQ ID NO: 18)

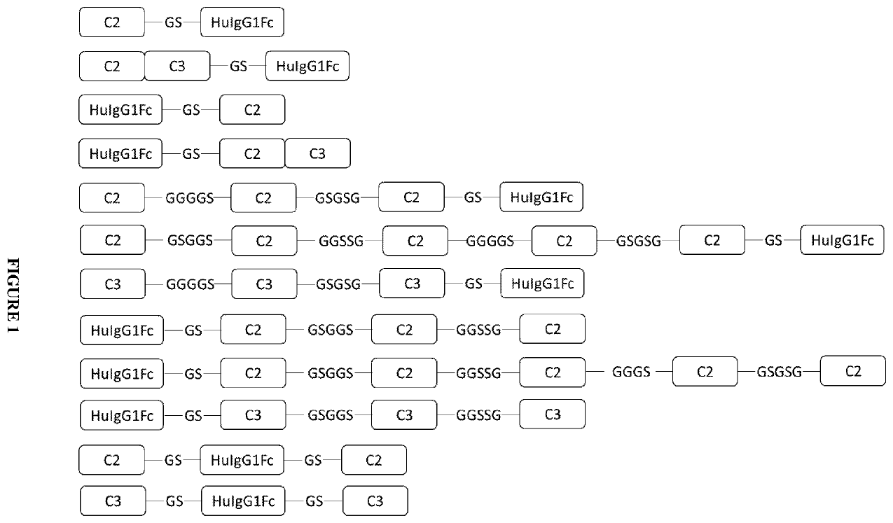

Recombinant proteins were expressed using the ExpiCHO™ transient expression system. A schematic representation of the proteins is shown in FIG. 1.

SDS-PAGE gel analysis of the recombinant envelope-binding proteins constructs comprising a single CR2, CR3 or CR2-CR3 domain under reduced and non-reduced conditions were well expressed.

To increase avidity, constructs comprising multiple CR2 and/or CR3 domains were produced. SDS-PAGE gel analysis under reduced and non-reduced conditions showed higher order species indicative of aggregation. Lower yield compared to single domain constructs was also observed.

To further increase avidity, CR2 or CR3 domains were fused to both the C-terminus and N-terminus of the IgG1 Fc.

All proteins were purified using protein A chromatography.

### Example 2: Quantification of Binding of Vesicular Stomatitis Virus G (VSV-G) to Recombinant Envelope-Binding Proteins

Binding of human IgG1Fc tagged C2C3 LDLR concatemers to vesicular stomatitis virus G (VSV-G) (ProteoGenix) was analysed using surface plasmon resonance (SPR).

Briefly, Biacore T200 and 8K+ were used for binding analysis. All sensor chips were normalized according to the manufacturer's directions prior to use. All buffers and solutions were filtered (0.22 μm) prior to use. Assays were performed at 37° C. using 1×TBS (supplemented with or without 5 mM CaCl₂) as running buffer in acidic (6.0) and neutral (7.4) pH.

Recombinant envelope-binding proteins and human IgG1 Fc control were buffer exchanged into running buffer 1×TBS (pH 7.4) after treating with 10 mM EDTA to remove calcium ions bound during the purification stages. Equal volumes of proteins (stock concentration) and 20 mM EDTA were mixed and incubated at room temperature for 60 minutes. Samples were then buffer exchanged into running buffer (1×TBS) supplemented with or without 5 mM CaCl₂) and the concentration was measured using A280 with respective extension co-efficient (Table 1).

TABLE 1
Concentrations of all the envelope-binding proteins after
buffer exchange were measured using A280 absorbance
using their respective extinsion co-efficient (E1%)

| Envelope-binding | Extinsion co-efficient | Absorbance | Concentration |
|---|---|---|---|
| protein | (E_{1%}) | (A_{Z80}) | (mg/mL) |
| C2 Fc | 1.36 | 14.99 | 11.03 |
| Fc C2 | 1.36 | 17.61 | 12.96 |
| C2C3 Fc | 1.20 | 10.83 | 9.01 |
| Fc C2C3 | 1.20 | 14.11 | 11.74 |
| Fc | 1.37 | 17.29 | 12.60 |
| C2FcC2 | 1.35 | 1.52 | 1.08 |
| C3FcC3 | 1.04 | 3.86 | 3.51 |

Qualitative binding analysis was performed. Anti-VSVG mouse monoclonal antibody was directly immobilised onto the carboxymethyl dextran surface of a CM5 sensorchip to approximately ~16,000 RU using standard NHS/EDC chemistry. VSVG expressed as exosomes was stably captured from cell culture supernatants on the active flow cell for 10 minutes. Capture levels were approximately 1200 RU. Reference surface was injected with cell culture supernatants containing non-VSVG expressing exosomes.

Buffer exchanged envelope-binding proteins and the control Fc were over all flow cells at a single concentration of 5 pM to assess the binding levels. Surface was regenerated with a 60 second injection of 350 mM EDTA in between injections.

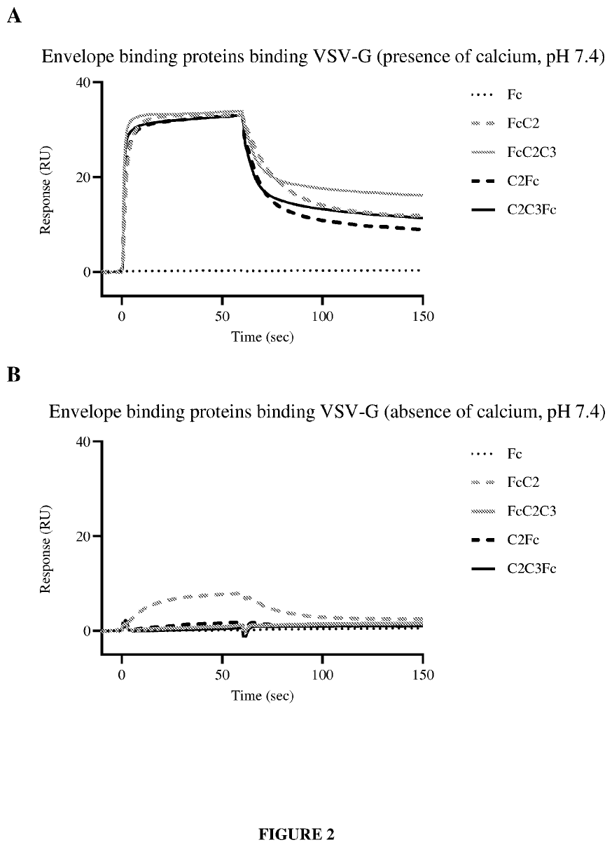

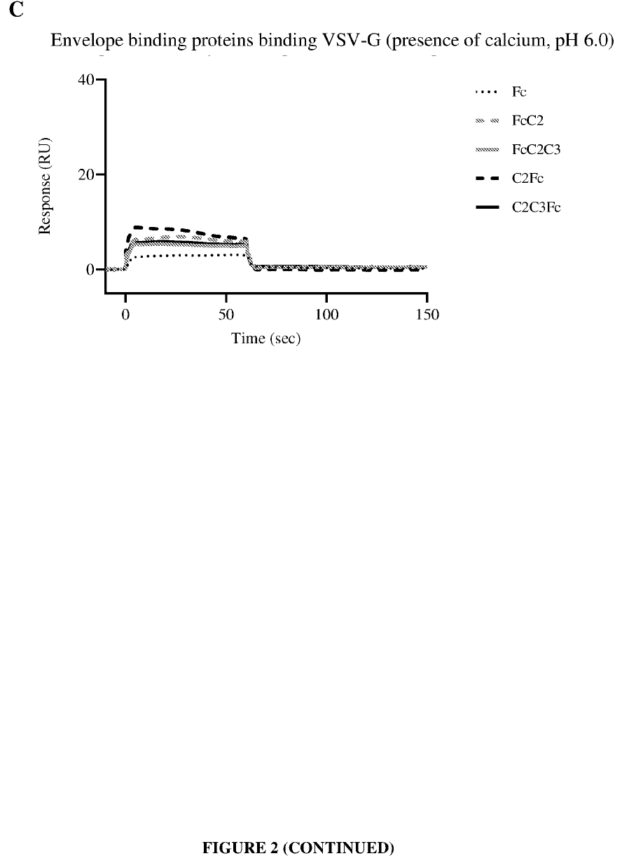

As shown in FIG. 2 clear binding of VSVG to all envelope-binding proteins were observed in presence of Ca2+ ions at neutral pH (Table 2; FIG. 2A). No or minimal binding observed in the absence of Ca2+ ions (Table 2; FIG. 2B). No binding of envelope-binding proteins was observed in acidic pH even in the presence of Ca2+ ions (Table 2; FIG. 2C).

TABLE 2
Comparative binding levels in response units (RU) of recombinant
envelope-binding proteins injected at 5 μM over the VSVG
captured surface (2000 RU) across all buffer conditions

| | VSVG 5 μM binding in Response Units (RU) | | |
|---|---|---|---|
| | 1X TBS, | 1X TBS, | 1X TBS, |
| LDL receptor | 5 mM CaCl₂ pH 7.4 | pH 7.4 | 5 mM CaCl₂ pH 6.0 |
| Fc C2 | 30 | 7 | NSB |
| C2 Fc | 35 | NSB | NSB |
| Fc C2C3 | 30 | NSB | NSB |
| C2C3 Fc | 30 | NSB | NSB |

NSB: No significant binding observed

Affinity analysis was done on a fresh CM5 chip similarly immobilised with Anti-VSVG mouse monoclonal antibody. VSVG expressing exosomes were captured to ~2000 RU from a new batch of cell culture supernatants. Recombinant envelope-binding proteins were injected in a concentration range in 2-fold dilution series (i.e., 5 μM, 2.5 μM, 1.25 μM, 0.625 μM, and 0.3125 μM) prepared in running buffer over all flow cells.

Surface was regenerated with a 60 second injection of 350 mM EDTA in between sample injections.

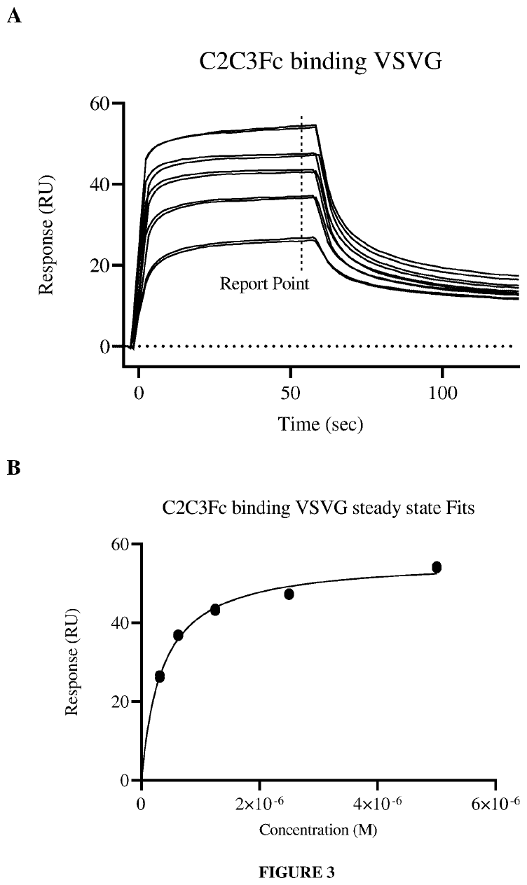

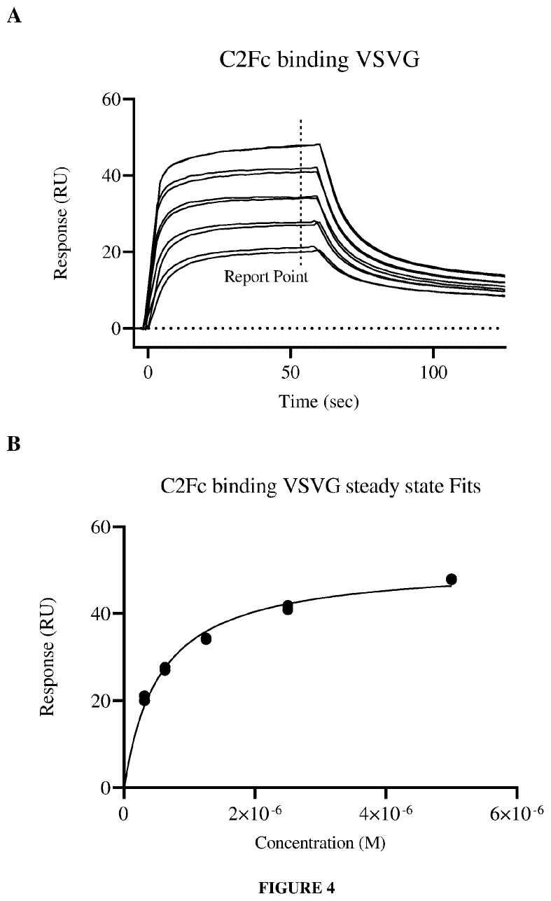

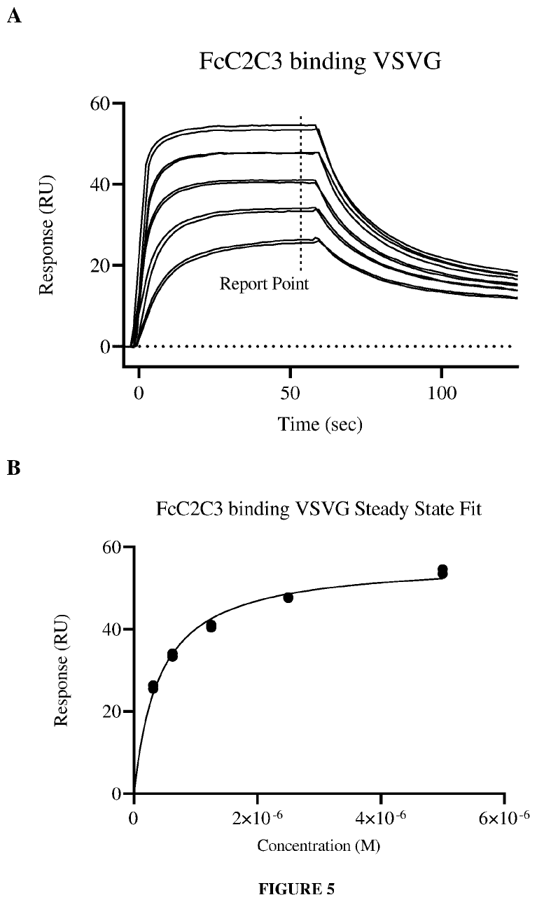

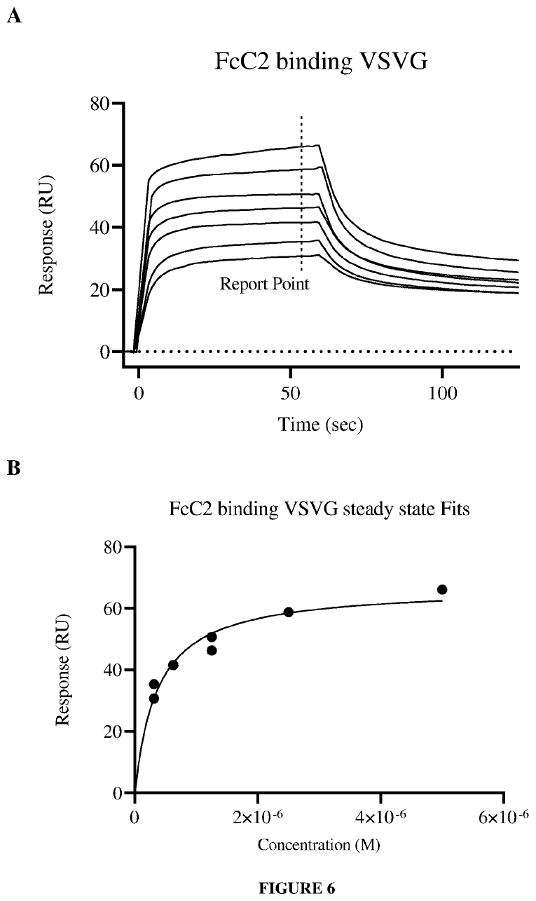

As shown in Table 3 and FIGS. 3-6 an apparent steady state affinity for each interaction was measured (with the topmost curve corresponding to the highest concentration (i.e., 5 μM) and bottommost curve the lowest concentration (i.e., 0.3125 μM). VSV-G exhibited similar range of affinities for all the recombinant envelope-binding proteins tested.

TABLE 3
Calculated apparent steady state binding affinities of
recombinant envelope-binding proteins binding VSV-G expressed
as exosomes. KD values were calculated from sensorgram
data fit to a 1:1 state binding model N = 1 run.

| Analytes | KD (nM) | Rmax (RU) | Chi² (RU²) |
|---|---|---|---|
| C2 Fc | 534.2 | 51.38 | 2.36 |
| Fc C2 | 374.1 | 67.57 | 11.4 |
| C2C3 Fc | 352.0 | 56.14 | 2.05 |
| Fc C2C3 | 412.5 | 56.53 | 2.67 |

### Example 3: Visualisation of Binding of Recombinant Envelope-Binding Proteins to VSV-G Expressing Cells

VSV-G was expressed in GPRG cells using an inducible promoter and flow cytometry was used to visualise binding of the recombinant envelope-binding protein constructs to VSV-G expressing GPRG cells and to non-VSV-G expressing 293T cells. An anti-VSV-G antibody was used as a positive control to show that induced GPRG cells have high levels of VSV-G expression compared to 293T cells.

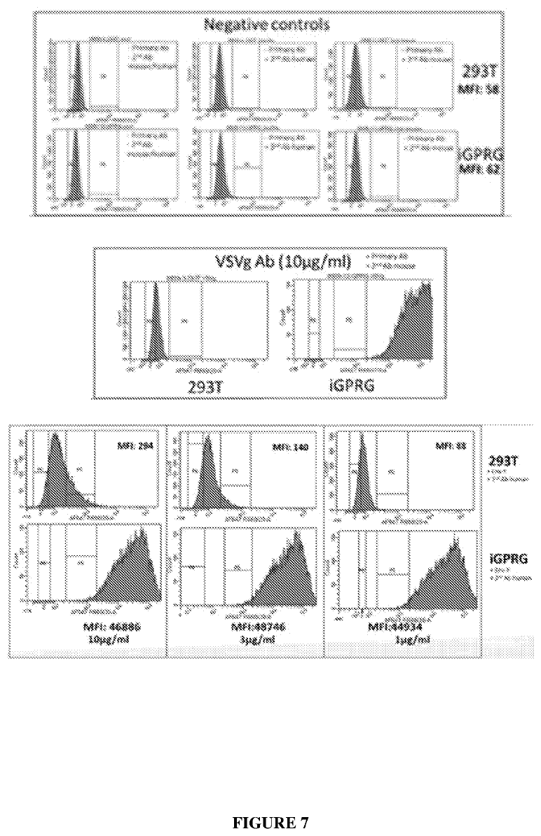

Binding affinity of four recombinant envelope-binding protein constructs (i.e., Fc-C2, Fc-C2-C3, C2-Fc, and C2-C3-Fc) was analysed at increasing protein concentrations (10 μg/ml, 3 μg/ml, and 1 μg/ml). All four recombinant envelope-binding protein constructs showed specific binding to VSV-G expressing GPRG cells, compared to 293T cells. FIG. 7 shows data for C2-Fc binding, however similar binding was also observed for the remaining proteins analysed. Lower concentrations of protein showed low levels of non-specific binding to HEK 293T cells.

### Example 4: Binding Dissociation of Recombinant Envelope-Binding Protein Constructs to VSV-G Expressing Cells

Binding dissociation of the recombinant envelope-binding protein constructs to VSV-G expressing cells was investigated.

Protein (at 1 μg/ml) was added to VSV-G expressing GPRG cells and incubated at room temperature for 30 minutes to allow for protein binding to VSV-G. Subsequently, the cells were washed with PBS pH7.0 twice and then treated with 1 of 5 treatment conditions as follows:
1. PBS, pH7 (control Ca²⁺)
2. DPBS, pH7 (Ca²⁺-free)
3. DPBS, pH7+10 mM EDTA
4. DPBS, pH6 (Ca²⁺-free)
5. DPBS, pH6+10 mM EDTA

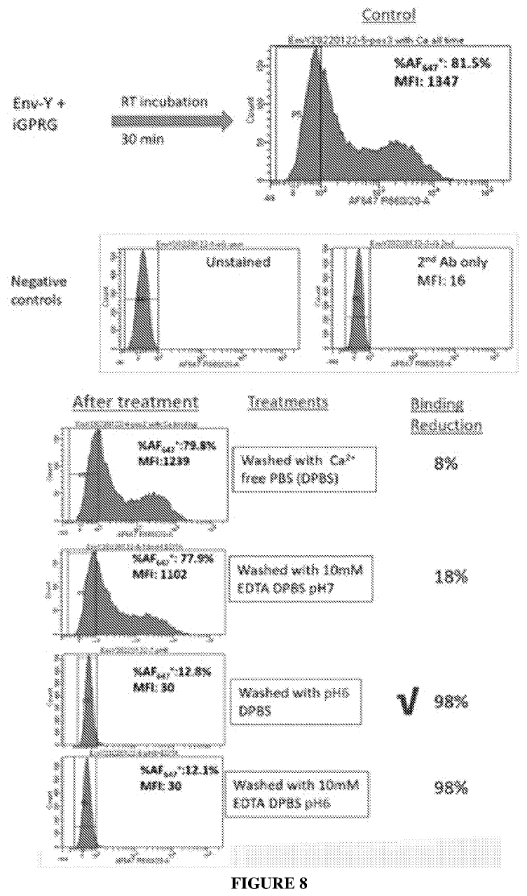

Cell binding was then visualised by flow cytometry as shown in FIG. 8. Treatment with Ca²⁺ free DPBS pH7 showed a slight reduction in binding by 8%. Treatment with 10 mM EDTA DPBS pH7 showed slightly increased dissociation at 18%. Treatment with pH6 DPBS showed the largest reduction in binding, with 98% dissociation between proteins and VSV-G expressing GPRG cells. The addition of 10 mM EDTA to pH6 DPBS did not increase the binding dissociation showing that the bound envelope-binding proteins can be dissociated with DPBS at pH6.

### Example 5: Transduction Inhibition by Recombinant Envelope-Binding Proteins

A neutralisation study using flow cytometry was performed to quantify the amount of recombinant envelope-binding protein construct required to block transduction of HEK293 cells with GFP lentiviral vector.

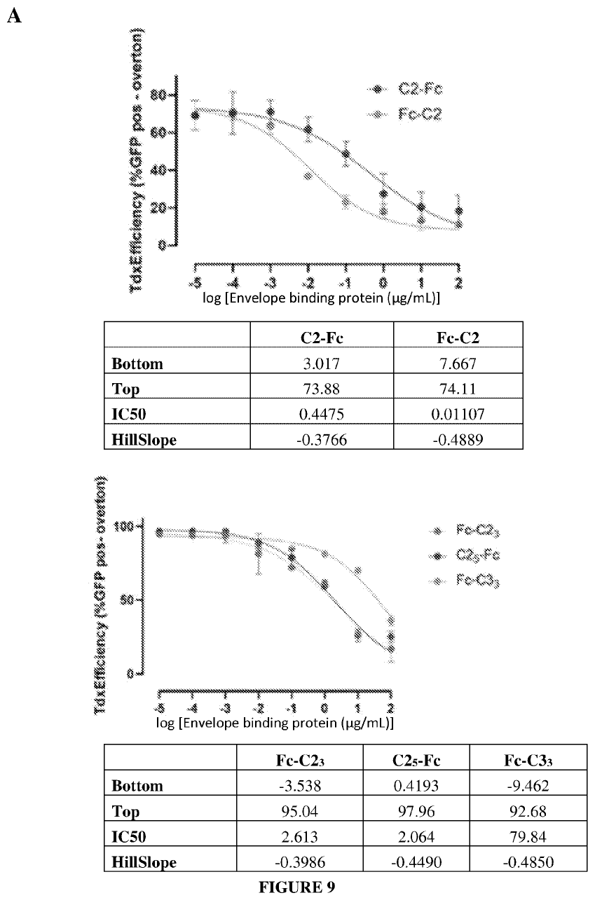

As shown in FIG. 9, recombinant envelope-binding protein constructs C2-Fc and Fc-C2 were the most effective at reducing transduction, with a broad dynamic range. Table 4 shows the IC₅₀ of all recombinant envelope-binding protein constructs tested, with constructs comprising the CR2 domain more effective at blocking transduction.

TABLE 4
IC₅₀ of recombinant envelope-binding protein
constructs for neutralisation of transduction
of HEK293 cells with GFP lentiviral vector

| Recombinant envelope-binding | IC₅₀ |
|---|---|
| protein construct | (μg/mL) |
| Fc—C2 | 0.011 |
| C2—Fc | 0.45 |
| C2₅—Fc | 2.1 |
| Fc—C2₃ | 2.6 |
| C2—C3—Fc | 9.3 |
| Fc—C2—C3 | 17 |
| Fc—C3₃ | 80 |

### Example 6: Production of Lentiviral Vector (LVV) Using Recombinant Envelope-Binding Protein Constructs

To investigate whether the recombinant envelope-binding protein constructs increased lentiviral vector yield during cell culture LVV expressing cells were cultured in the presence of the recombinant envelope-binding protein constructs. Initially, cells were seeded at a density of 8.5E4 cells/cm², and grown for 48 hours. Recombinant envelope-binding protein constructs (Fc-C2 and C2-Fc) were then added to the cell culture media at induction (i.e., day 0) and each day thereafter at six different concentrations (100, 10, 1, 0.1, 0.01, 0.001 μg/ml). Cells were harvested at days 2, 3, and 4.

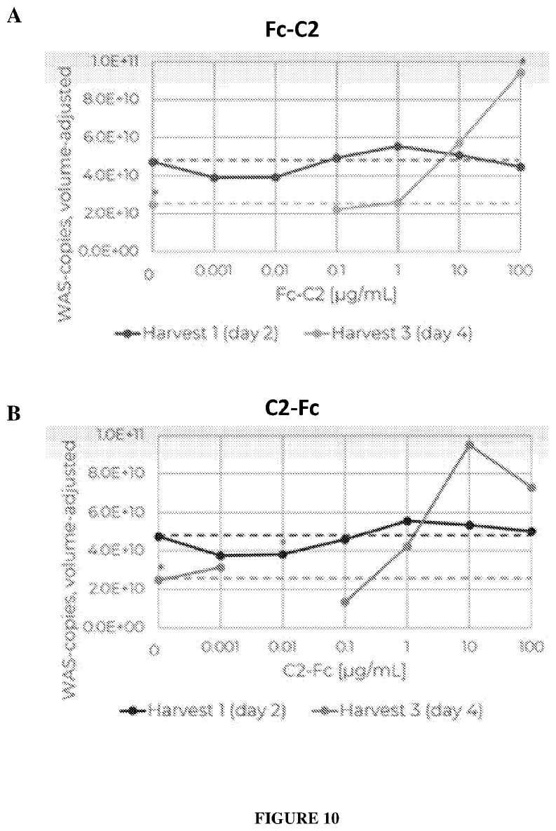

Although no effect on yield was observed when cells were harvested at day 2, the physical virus titer increased by 3.8 fold when recombinant envelope-binding protein constructs were added at higher concentrations (10 and 100 μg/ml), as compared to control, when harvested at day 4 (FIG. 10). These data show that recombinant envelope-binding protein constructs can increase lentivirus yields.

### Example 7: Purification of LVV by Protein A Chromatography

To investigate whether recombinant envelope-binding protein constructs are dissociable from the lentivirus during downstream purification, recombinant envelope-binding protein-bound virus is loaded onto a Protein A column in the presence of 5 mM Ca²⁺, followed by elution at low Ca²⁺ and/or pH6.0.

### Example 8: Measuring the Kinetics and Affinity of Envelope-Binding Proteins to VSVG with and without Avidity Conditions

### Under Avidity Conditions

Anti-VSVG mouse monoclonal antibody was directly immobilised onto the carboxymethyl dextran surface of a CM5 sensorchip to approximately 16,000 RU using standard NHS/EDC chemistry. Recombinant VSVG or VSVG expressed as VLPs were stably captured on the active flow cell. Capture levels were approximately 200 and 1200 RU respectively. Flow cells with no VSVG captured was used as the reference. Complete regeneration of captured VSVG from the antibody surface was not achievable because of its tight binding and therefore, VSVG was considered tethered to the antibody surface. Envelope-binding proteins were injected in a concentration range (50-1.56 nM) in 2-fold dilution over all flow cells. Association was done for 60 s for VLP captured surface and 120 s for recombinant VSVG captured surface. Dissociation was monitored for 1200 s. The VSVG surfaces (recombinant and as VLPs) were regenerated with an 8 second injection of GEB in between sample injections.

Recombinantly made full length (25-788 amino acid long) LDLR protein (R&D systems) was also used in this assay to compare both forms of VSVG (recombinant and VLPs). Full length LDLR protein is also used to compare with LDL-R fragmented proteins for its binding affinities to VSVG. LDLR was injected over a freshly made VSVG tethered surface in a concentration range (100 to 3.12 nM) in 2-fold dilution series. VSVG tethered surface was regenerated with an 8 second injection of GEB in between sample injections.

The KDs are reported as apparent affinities (because of avidity component) and are purely qualitative. All proteins except C2Fc bound VSVG expressed as VLPs with comparable affinities calculated in the range of 200 to 300 pM (Table 5). C2Fc bound with a comparative weaker affinity of ~1 nM. C2FcC2 and C3FcC3 bound recombinant VSVG with approximate affinities of 391 and 537 pM respectively (Table 6). FcC2 bound recombinant VSVG with an affinity of 219 pM, whereas affinity measurement was not possible for C2Fc because of the low binding signal.

Full length LDL-R protein bound VSVG (recombinant and VLPs) with a relatively weaker affinity of ~1.0 and 0.73 nM respectively, compared to LDLR fragmented proteins (Table 7).

TABLE 5
Calculated kinetics and apparent binding affinities (with an avidity
component) for envelope-binding proteins binding VSVG expressed as
exosomes. K_{D} values were calculated from sensorgram data fitted to
a 1:1 affinity binding model from N = 6 replicates.

| Analyte | kₐ (1/Ms) | k_{d} (1/s) | K_{D} (pM) |
|---|---|---|---|
| C2FcC2 | 6.0 × 10⁵ | 1.7 × 10⁻⁴ | 284.6 ± 15.0 |
| C3FcC3 | 9.6 × 10⁵ | 3.0 × 10⁻⁴ | 312.0 ± 1.0 |
| C2Fc | 1.1 × 10⁶ | 1.1 × 10⁻³ | 1011.0 ± 91.8 |
| FcC2 | 4.2 × 10⁵ | 1.2 × 10⁻⁴ | 291.0 ± 3.5 |
| C2C3Fc | 1.5 × 10⁵ | 4.5 × 10⁻⁴ | 310.6 ± 4.3 |
| FcC2C3 | 9.7 × 10⁵ | 1.7 × 10⁻⁴ | 213.0 ± 0.11 |

TABLE 6
Calculated rate constants and apparent binding affinities (with an
avidity component) for envelope-binding protein variants binding
recombinant VSVG. K_{D} indicated as Mean ± SEM from sensorgram
data fitted to a 1:1 affinity binding model from N = 3 replicates.

| Analyte | kₐ (1/Ms) | k_{d} (1/s) | K_{D} (pM) |
|---|---|---|---|
| C2FcC2 | 1.5 × 10⁶ | 5.7 × 10⁻⁴ | 391.3 ± 19.0 |
| C3FcC3 | 1.9 × 10⁶ | 9.8 × 10⁻⁴ | 537.0 ± 50.4 |

| | |
|---|---|
| C2Fc | Low binding level |

| | | | |
|---|---|---|---|
| FcC2 | 1.2 × 10⁶ | 2.7 × 10⁻⁴ | 219.5 ± 20.1 |

TABLE 7
Full length recombinant LDLR bound VSVG (recombinant and
VLP) with comparable apparent affinities in the range
of 700 to 1000 pM. Affinity measurements against VLPs are estimates
only due to low binding signals (RU). K_{D} indicated as
Mean ± SEM from sensorgram data fitted to a 1:1
affinity binding model from N = 3 replicates.

| Ligand | kₐ (1/Ms) | k_{d} (1/s) | K_{D} (pM) |
|---|---|---|---|
| Recombinant VSVG | 8.0 × 10⁵ | 8.8 × 10⁻⁴ | 1116.7 ± 48.4 |
| VSVG as VLPs | 6.6 × 10⁵ | 4.4 × 10⁻⁴ | 726.0 ± 105.6 |

### Without Avidity

Protein G sensor chip was used for the analysis. Envelope-binding proteins were diluted to 2 μg/mL in running buffer and captured on the active flow cells for 60 s. Capture levels were ~500 RU. Recombinant VSVG was injected over all flow cells for 150 seconds in a concentration range (50-1.56 nM) in 2-fold dilution series. Dissociation was monitored for 1200 seconds. Sensor surface was regenerated with 60 second injection of 10 mM glycine, pH 1.5 in between cycles.

Recombinant VSVG bound all the envelope-binding protein variants, except C2Fc with comparable affinities in the range of 800 to 1000 pM (Table 8). A low binding signal (RU) was observed for C2Fc.

TABLE 8
Recombinant VSVG bound envelope-binding proteins with a comparable
affinity in the range of 700 to 900 pM, except there were no affinity
measurements derived for C2Fc binding because of low response levels.
K_{D} indicated as Mean ± SEM from sensorgram data fitted to
a 1:1 affinity binding model from N = 3 replicates.

| Ligand | kₐ (1/Ms) | k_{d} (1/s) | K_{D} (pM) |
|---|---|---|---|
| C2FcC2 | 2.9 × 10⁵ | 2.8 × 10⁻⁴ | 973.6 ± 70.1 |
| C3FcC3 | 2.2 × 10⁵ | 1.6 × 10⁻⁴ | 769.7 ± 105.2 |

| | |
|---|---|
| C2Fc | Low binding level |

| | | | |
|---|---|---|---|
| FcC2 | 3.5 × 10⁵ | 3.0 × 10⁻⁴ | 876.3 ± 72.8 |

1. An envelope-binding protein comprising one or more low-density lipoprotein receptor (LDLR) domains and a purification tag.

2. The envelope-binding protein of claim 1, wherein the envelope-binding protein:
(i) specifically binds vesicular stomatitis virus G (VSV-G) pseudotyped virus;
(ii) specifically binds VSV-G pseudotyped virus in the presence of calcium;
(iii) specifically binds VSV-G pseudotyped virus in the presence of at least about 0.01 mM calcium; or
(iv) specifically binds VSV-G pseudotyped virus in the presence of at least about 5 mM calcium.

3-5. (canceled)

6. The envelope-binding protein of claim 2, wherein:
(i) the envelope-binding protein binds VSV-G pseudotyped virus at a pH of between pH 6.5 and pH 9.0; and/or
(ii) binding of the envelope-binding protein to VSV-G pseudotyped virus dissociates at pH below 6.5.

7. (canceled)

8. The envelope-binding protein of claim 1, wherein the one or more LDLR domains comprise a LDLR extracellular domain, wherein the LDLR extracellular domain comprises a ligand binding domain, an epidermal growth factor (EGF) precursor homology domain, and/or an O-linked sugar domain, wherein the ligand binding domain comprises one or more cysteine-rich complement-type repeat (CR) domains, and wherein the one or more CR domains are a complement-type repeat 1 (CR1) domain, a complement-type repeat 2 (CR2) domain, a complement-type repeat 3 (CR3) domain, a complement-type repeat 4 (CR4) domain, a complement-type repeat 5 (CR5) domain, a complement-type repeat 6 (CR6) domain, and/or a complement-type repeat 7 (CR7) domain.

9-11. (canceled)

12. The envelope-binding protein of claim 1, wherein:
(A) the envelope-binding protein comprises two or more LDLR domains directly or indirectly linked to each other, wherein the two or more LDLR domains are indirectly linked to each other via a linker, wherein the linker is a peptide linker comprising at least 2 amino acids in length, and
wherein the linker is selected from the group consisting of: a GS linker; a GSGGS linker; a GGSSG linker; a GGGGS linker; a GSGSG linker; a (Gly)₈ linker; a (Gly)₆ linker; a (GGGS)ₙ linker, wherein n=1, 2, 3 or 4: a (EAAAK)ₙ linker, wherein n=1, 2 or 3: a A(EAAAK)₄ALEA(EAAAK4)A linker; a AEAAAKEAAAKA linker; a (Ala-Pro)ₙ linker, wherein n=10-34; and a PAPAP linker; and/or
(B) (i) the purification tag is directly linked to the one or more LDLR domains; or
(ii) the purification tag is indirectly linked to the one or more LDLR domains via a linker, wherein the linker is a peptide linker comprising at least 2 amino acids in length, and
wherein the linker is selected from the group consisting of: a GS linker; a GSGGS linker; a GGSSG linker; a GGGGS linker; a GSGSG linker; a (Gly)₈ linker; a (Gly)₆ linker; a (GGGS)ₙ linker, wherein n=1, 2, 3 or 4: a (EAAAK)ₙ linker, wherein n=1, 2 or 3: a A(EAAAK)₄ALEA(EAAAK₄)A linker; a AEAAAKEAAAKA linker; a (Ala-Pro)ₙ linker, wherein n=10-34; and a PAPAP linker.

13-19. (canceled)

20. The envelope-binding protein of claim 1, wherein the envelope-binding protein comprises, in order from N- to C-terminus:
(i) the purification tag indirectly or directly linked to a LDLR domain;
(ii) a LDLR domain indirectly or directly linked to the purification tag; or
(iii) a first LDLR domain, the purification tag, and a second LDLR domain, wherein each of the first and/or second LDLR domains and the purification tag are indirectly or directly linked;
optionally, wherein the envelope-binding protein comprises one or more additional LDLR domains indirectly or directly linked to the N- and/or C-terminus.

21. (canceled)

22. The envelope-binding protein of claim 1, wherein:
(i) the one or more LDLR domains are a CR1 LDLR domain, a CR2 LDLR domain, a CR3 LDLR domain, a CR4 LDLR domain, a CR5 LDLR domain, a CR6 LDLR domain, and/or a CR7 LDLR domain; or
(ii) the one or more LDLR domains are a CR2 LDLR domain and/or a CR3 LDLR domain;
wherein the envelope-binding protein comprises five or fewer CR2 LDLR domains; and/or five or fewer CR3 LDLR domains.

23-24. (canceled)

25. The envelope-binding protein of claim 22, wherein the envelope-binding protein comprises, in order from N- to C-terminus:
(i) the purification tag indirectly or directly linked to a CR2 LDLR domain;
(ii) a CR2 LDLR domain indirectly or directly linked to the purification tag;
(iii) a CR3 LDLR domain indirectly or directly linked to the purification tag;
(iv) the purification tag indirectly or directly linked to a CR3 LDLR domain;
(v) a first CR2 LDLR domain, the purification tag, and a second CR2 LDLR domain, wherein each of the CR2 LDLR domains and the purification tag are indirectly or directly linked;
(vi) a first CR3 LDLR domain, the purification tag, and a second CR3 LDLR domain, wherein each of the CR3 LDLR domains and the purification tag are indirectly or directly linked;
(vii) a CR2 LDLR domain, the purification tag, and a CR3 LDLR domain, wherein the CR2 and CR3 LDLR domains and the purification tag, are indirectly or directly linked; or
(viii) a CR3 LDLR domain, the purification tag, and a CR2 LDLR domain, wherein the CR2 and CR3 LDLR domains and the purification tag are indirectly or directly linked;
optionally, wherein the envelope-binding protein comprises one or more additional CR2 and/or CR3 LDLR domains indirectly or directly linked to the N- and/or C-terminus.

26-27. (canceled)

28. The envelope-binding protein of claim 22, wherein the envelope-binding protein comprises, in order from N- to C-terminus:
(i) a CR1 LDLR domain, a CR2 LDLR domain, a CR3 LDLR domain, a CR4 LDLR domain, a CR5 LDLR domain, a CR6 LDLR domain, and a CR7 LDLR domain indirectly or directly linked to the purification tag; or
(ii) the purification tag indirectly or directly linked to a CR1 LDLR domain, a CR2 LDLR domain, a CR3 LDLR domain, a CR4 LDLR domain, a CR5 LDLR domain, a CR6 LDLR domain, and a CR7 LDLR domain.

29. The envelope-binding protein of claim 1, wherein the purification tag is a protein or peptide tag, wherein the protein or peptide tag is selected from the group consisting of an immunoglobulin Fc, albumin, a hexahistidine tag, a human influenza hemaglutinin (HA) tag, a FLAG-tag, a calmodulin binding peptide (CBP) tag, a poly-glutamate tag, a polycysteine (Cys) tag, a polyhistidine (His) tag, a Myc-tag, a streptavidin-binding peptide (SBP) tag, a streptavidin (Strep) tag, an avidin tag, a bacteriophage V5 epitope (V5) tag, an isopeptag, a SpyTag, a biotin-carboxyl carrier protein (BCCP) tag, a Halo-tag, a thioredoxin (Trx) tag, a small ubiquitin-like molecule (SUMO) tag, and a maltose binding protein (MBP) tag, and wherein the purification tag is not a glutathione S transferase (GST) tag.

30-31. (canceled)

32. The envelope-binding protein of claim 29, wherein the envelope-binding protein comprises, in order from N- to C-terminus:
(A)
(i) the immunoglobulin Fc indirectly or directly linked to a CR2 or CR3 LDLR domain;
(ii) a CR2 or CR3 LDLR domain indirectly or directly linked to the immunoglobulin F; or
(iii) a CR2 or CR3 LDLR domain, the immunoglobulin Fc, and a second CR2 or CR3 LDLR domain, wherein each of the CR2 and/or CR3 LDLR domains and the immunoglobulin Fc are indirectly or directly linked;
(B)
(i) the immunoglobulin Fc indirectly or directly linked to a CR2 LDLR domain;
(ii) a CR2 LDLR domain indirectly or directly linked to the immunoglobulin Fc;
(iii a CR3 LDLR domain indirectly or directly linked to the immunoglobulin Fc;
(iv) the immunoglobulin Fc indirectly or directly linked to a CR3 LDLR domain;
(v) a first CR2 LDLR domain, the immunoglobulin Fc, and a second CR2 LDLR domain, wherein each of the first and second CR2 LDLR domains and the immunoglobulin Fc are indirectly or directly linked;
(vi) a first CR3 LDLR domain, the immunoglobulin Fc, and a second CR3 LDLR domain, wherein each of the first and second CR3 LDLR domains and the immunoglobulin Fc are indirectly or directly linked;
(vii) a CR2 LDLR domain, the immunoglobulin Fc, and a CR3 LDLR domain, wherein the CR2 and CR3 LDLR domains and the immunoglobulin Fc are indirectly or directly linked; or
(viii) a CR3 LDLR domain, the immunoglobulin Fc, and a CR2 LDLR domain, wherein the CR2 and CR3 LDLR domains and the immunoglobulin Fc are indirectly or directly linked; or
(C)
(i) a CR2 LDLR domain, a CR3 LDLR domain, and the immunoglobulin Fc, wherein the CR2 and CR3 LDLR domains and the immunoglobulin Fc are indirectly or directly linked;
(ii) the immunoglobulin Fc, a CR2 LDLR domain, and a CR3 LDLR domain, wherein the immunoglobulin Fc and the CR2 and CR3 LDLR domains are indirectly or directly linked;
(iii) a first CR2 LDLR domain, a second CR2 LDLR domain, a third CR2 LDLR domain, and the immunoglobulin Fc, wherein the first, second, and third CR2 LDLR domains and the immunoglobulin are indirectly or directly linked;
(iv) a first CR2 LDLR domain, a second CR2 LDLR domain, a third CR2 LDLR domain, a fourth CR2 LDLR domain, a fifth CR2 LDLR domain, and the immunoglobulin Fc, wherein the first, second, third, fourth, and fifth CR2 LDLR domains and the immunoglobulin are indirectly or directly linked;
(v) a first CR3 LDLR domain, a second CR3 LDLR domain, a third CR3 LDLR domain, and the immunoglobulin Fc, wherein the first, second, and third CR3 LDLR domains and the immunoglobulin are indirectly or directly linked;
(vi) the immunoglobulin Fc, a first CR2 LDLR domain, a second CR2 LDLR domain, and a third CR2 LDLR domain, wherein the immunoglobulin and the first and second CR2 LDLR domains are indirectly or directly linked;
(vii) the immunoglobulin Fc, a first CR2 LDLR domain, a second CR2 LDLR domain, a third CR2 LDLR domain, a fourth CR2 LDLR domain, and a fifth CR2 LDLR domain, wherein the immunoglobulin and the first, second, third, fourth, and fifth CR2 LDLR domains are indirectly or directly linked;
(viii) the immunoglobulin Fc, a first CR3 LDLR domain, a second CR3 LDLR domain, and a third CR3 LDLR domain, wherein the immunoglobulin and the first, second, and third CR3 LDLR domains are indirectly or directly linked;
(ix) a first CR2 LDLR domain, the immunoglobulin Fc, and a second CR2 LDLR domain, wherein the immunoglobulin and the first and second CR2 LDLR domains are indirectly or directly linked; or
(x) a first CR3 LDLR domain, the immunoglobulin Fc, a second CR3 LDLR domain, wherein the immunoglobulin and the first and second CR3 LDLR domains are indirectly or directly linked.

33-36. (canceled)

37. The envelope-binding protein of claim 32, wherein:
(i) the CR2 LDLR domain comprises the sequence set forth in SEQ ID NO: 2;
(ii) the CR3 LDLR domain comprises the sequence set forth in SEQ ID NO: 3; and/or
(iii) the immunoglobulin Fc is an IgG1 Fc, wherein the IgG1 Fc comprises the sequence set forth in SEQ ID NO: 4

38-39. (canceled)

40. The envelope-binding protein of claim 29, wherein the envelope-binding protein comprises, or consists of, in order from N- to C-terminus:
(A)
(i) the immunoglobulin Fc comprising the sequence set forth in SEQ ID NO: 4, a GS linker, and a CR2 LDLR domain comprising amino acids 65 (±1 to 5 amino acids) to 105 (±1 to 5 amino acids) of SEQ ID NO: 1;
(ii) a CR2 LDLR domain comprising amino acids 65 (±1 to 5 amino acids) to 105 (±1 to 5 amino acids) of SEQ ID NO: 1, a GS linker, and the immunoglobulin Fc comprising the sequence set forth in SEQ ID NO: 4;
(iii) a CR2 LDLR domain comprising amino acids 65 (±1 to 5 amino acids) to 105 (±1 to 5 amino acids) of SEQ ID NO: 1, a CR3 LDLR domain comprising amino acid residues 106 (±1 to 5 amino acids) to 144 (±1 to 5 amino acids) of SEQ ID NO: 1, a GS linker, and the immunoglobulin Fc comprising the sequence set forth in SEQ ID NO: 4;
(iv) the immunoglobulin Fc comprising the sequence set forth in SEQ ID NO: 4, a GS linker, a CR2 LDLR domain comprising amino acids 65 (±1 to 5 amino acids) to 105 (±1 to 5 amino acids) of SEQ ID NO: 1, and a CR3 LDLR domain comprising amino acid residues 106 (±1 to 5 amino acids) to 144 (±1 to 5 amino acids) of SEQ ID NO: 1;
(v) a first CR2 LDLR domain comprising amino acids 65 (±1 to 5 amino acids) to 105 (±1 to 5 amino acids) of SEQ ID NO: 1, a GSGGS linker, a second CR2 LDLR domain comprising amino acids 65 (±1 to 5 amino acids) to 105 (±1 to 5 amino acids) of SEQ ID NO: 1, a GGSSG linker, a third CR2 LDLR domain comprising amino acids 65 (±1 to 5 amino acids) to 105 (±1 to 5 amino acids) of SEQ ID NO: 1, a GS linker, and the immunoglobulin Fc comprising the sequence set forth in SEQ ID NO: 4;
(vi) a first CR2 LDLR domain comprising amino acids 65 (±1 to 5 amino acids) to 105 (±1 to 5 amino acids) of SEQ ID NO: 1, a GSGGS linker, a second CR2 LDLR domain comprising amino acids 65 (±1 to 5 amino acids) to 105 (±1 to 5 amino acids) of SEQ ID NO: 1, a GGSSG linker, a third CR2 LDLR domain comprising amino acids 65 (±1 to 5 amino acids) to 105 (±1 to 5 amino acids) of SEQ ID NO: 1, a GGGGS linker, a fourth CR2 LDLR domain comprising amino acids 65 (±1 to 5 amino acids) to 105 (±1 to 5 amino acids) of SEQ ID NO: 1, a GSGSG linker, a fifth CR2 LDLR domain comprising amino acids 65 (±1 to 5 amino acids) to 105 (±1 to 5 amino acids) of SEQ ID NO: 1, and the immunoglobulin Fc comprising the sequence set forth in SEQ ID NO: 4;
(vii) a first CR3 LDLR domain comprising amino acid residues 106 (±1 to 5 amino acids) to 144 (±1 to 5 amino acids) of SEQ ID NO: 1, a GSGGS linker, a second CR3 LDLR domain comprising amino acid residues 106 (±1 to 5 amino acids) to 144 (±1 to 5 amino acids) of SEQ ID NO: 1, a GGSSG linker, a third CR3 LDLR domain comprising amino acid residues 106 (±1 to 5 amino acids) to 144 (±1 to 5 amino acids) of SEQ ID NO: 1, a GS linker, and the immunoglobulin Fc comprising the sequence set forth in SEQ ID NO: 4;
(viii) the immunoglobulin Fc comprising the sequence set forth in SEQ ID NO: 4, a GS linker, a first CR2 LDLR domain comprising amino acids 65 (±1 to 5 amino acids) to 105 (±1 to 5 amino acids) of SEQ ID NO: 1, a GSGGS linker, a second CR2 LDLR domain comprising amino acids 65 (±1 to 5 amino acids) to 105 (±1 to 5 amino acids) of SEQ ID NO: 1, a GGSSG linker, and a third CR2 LDLR domain comprising amino acids 65 (±1 to 5 amino acids) to 105 (±1 to 5 amino acids) of SEQ ID NO: 1;
(ix) the immunoglobulin Fc comprising the sequence set forth in SEQ ID NO: 4, a GS linker, a first CR2 LDLR domain comprising amino acids 65 (±1 to 5 amino acids) to 105 (±1 to 5 amino acids) of SEQ ID NO: 1, a GSGGS linker, a second CR2 LDLR domain comprising amino acids 65 (±1 to 5 amino acids) to 105 (±1 to 5 amino acids) of SEQ ID NO: 1, a GGSSG linker, a third CR2 LDLR domain comprising amino acids 65 (±1 to 5 amino acids) to 105 (±1 to 5 amino acids) of SEQ ID NO: 1, a GGGGS linker, a fourth CR2 LDLR domain comprising amino acids 65 (±1 to 5 amino acids) to 105 (±1 to 5 amino acids) of SEQ ID NO: 1, a GSGSG linker, and a fifth CR2 LDLR domain comprising amino acids 65 (±1 to 5 amino acids) to 105 (±1 to 5 amino acids) of SEQ ID NO: 1;
(x) the immunoglobulin Fc comprising the sequence set forth in SEQ ID NO: 4, a GS linker, a first CR3 LDLR domain comprising amino acid residues 106 (±1 to amino acids) to 144 (±1 to 5 amino acids) of SEQ ID NO: 1, a GSGGS linker, a second CR3 LDLR domain comprising amino acid residues 106 (±1 to 5 amino acids) to 144 (±1 to 5 amino acids) of SEQ ID NO: 1, a GGSSG linker, and a third CR3 LDLR domain comprising amino acid residues 106 (±1 to 5 amino acids) to 144 (±1 to 5 amino acids) of SEQ ID NO: 1;
(xi) a first CR2 LDLR domain comprising amino acids 65 (±1 to 5 amino acids) to 105 (±1 to 5 amino acids) of SEQ ID NO: 1, a GS linker, the immunoglobulin Fc comprising the sequence set forth in SEQ ID NO: 4, a GS linker, and a second CR2 LDLR domain comprising amino acids 65 (±1 to 5 amino acids) to 105 (±1 to 5 amino acids) of SEQ ID NO: 1; or
(xii) a first CR3 LDLR domain comprising amino acid residues 106 (±1 to 5 amino acids) to 144 (±1 to 5 amino acids) of SEQ ID NO: 1, a GS linker, the immunoglobulin Fc comprising the sequence set forth in SEQ ID NO: 4, a GS linker, and a second CR3 LDLR domain comprising amino acid residues 106 (±1 to 5 amino acids) to 144 (±1 to 5 amino acids) of SEQ ID NO: 1, or
(B)
(i) the immunoglobulin Fc comprising the sequence set forth in SEQ ID NO: 4, a GS linker, and a CR2 LDLR domain comprising the sequence set forth in SEQ ID NO: 2;
(ii) a CR2 LDLR domain comprising the sequence set forth in SEQ ID NO: 2, a GS linker, and the immunoglobulin Fc comprising the sequence set forth in SEQ ID NO: 4;
(iii) a CR2 LDLR domain comprising the sequence set forth in SEQ ID NO: 2, a CR3 LDLR domain comprising the sequence set forth in SEQ ID NO: 3, a GS linker, and the immunoglobulin Fc comprising the sequence set forth in SEQ ID NO: 4;
(iv) the immunoglobulin Fc comprising the sequence set forth in SEQ ID NO: 4, a GS linker, a CR2 LDLR domain comprising the sequence set forth in SEQ ID NO: 2, and a CR3 LDLR domain comprising the sequence set forth in SEQ ID NO: 3;
(v) a first CR2 LDLR domain comprising the sequence set forth in SEQ ID NO: 2, a GSGGS linker, a second CR2 LDLR domain comprising the sequence set forth in SEQ ID NO: 2, a GGSSG linker, a third CR2 LDLR domain comprising the sequence set forth in SEQ ID NO: 2, a GS linker, and the immunoglobulin Fc comprising the sequence set forth in SEQ ID NO: 4;
(vi) a first CR2 LDLR domain comprising the sequence set forth in SEQ ID NO: 2, a GSGGS linker, a second CR2 LDLR domain comprising the sequence set forth in SEQ ID NO: 2, a GGSSG linker, a third CR2 LDLR domain comprising the sequence set forth in SEQ ID NO: 2, a GGGGS linker, a fourth CR2 LDLR domain comprising the sequence set forth in SEQ ID NO: 2, a GSGSG linker, a fifth CR2 LDLR domain comprising the sequence set forth in SEQ ID NO: 2, and the immunoglobulin Fc comprising the sequence set forth in SEQ ID NO: 4;
(vii) a first CR3 LDLR domain comprising the sequence set forth in SEQ ID NO: 3, a GSGGS linker, a second CR3 LDLR domain comprising the sequence set forth in SEQ ID NO: 3, a GGSSG linker, a third CR3 LDLR domain comprising the sequence set forth in SEQ ID NO: 3, a GS linker, and the immunoglobulin Fc comprising the sequence set forth in SEQ ID NO: 4;
(viii) the immunoglobulin Fc comprising the sequence set forth in SEQ ID NO: 4, a GS linker, a first CR2 LDLR domain comprising the sequence set forth in SEQ ID NO: 2, a GSGGS linker, a second CR2 LDLR domain comprising the sequence set forth in SEQ ID NO: 2, a GGSSG linker, and a third CR2 LDLR domain comprising the sequence set forth in SEQ ID NO: 2;
(ix) the immunoglobulin Fc comprising the sequence set forth in SEQ ID NO: 4, a GS linker, a first CR2 LDLR domain comprising the sequence set forth in SEQ ID NO: 2, a GSGGS linker, a second CR2 LDLR domain comprising the sequence set forth in SEQ ID NO: 2, a GGSSG linker, a third CR2 LDLR domain comprising the sequence set forth in SEQ ID NO: 2, a GGGGS linker, a fourth CR2 LDLR domain comprising the sequence set forth in SEQ ID NO: 2, a GSGSG linker, and a fifth CR2 LDLR domain comprising the sequence set forth in SEQ ID NO: 2;
(x) the immunoglobulin Fc comprising the sequence set forth in SEQ ID NO: 4, a GS linker, a first CR3 LDLR domain comprising the sequence set forth in SEQ ID NO: 3, a GSGGS linker, a second CR3 LDLR domain comprising the sequence set forth in SEQ ID NO: 3, a GGSSG linker, and a third CR3 LDLR domain comprising the sequence set forth in SEQ ID NO: 3;
(xi) a first CR2 LDLR domain comprising the sequence set forth in SEQ ID NO: 2, a GS linker, the immunoglobulin Fc comprising the sequence set forth in SEQ ID NO: 4, a GS linker, and a second CR2 LDLR domain comprising the sequence set forth in SEQ ID NO: 2; or
(xii) a first CR3 LDLR domain comprising the sequence set forth in SEQ ID NO: 3, a GS linker, the immunoglobulin Fc comprising the sequence set forth in SEQ ID NO: 4, a GS linker, and a second CR3 LDLR domain comprising the sequence set forth in SEQ ID NO: 3.

41. (canceled)

42. The envelope-binding protein of claim 1, wherein:
(i) the envelope-binding protein comprises or consists of the sequence set forth in SEQ ID NO: 9, optionally wherein the protein lacks the signal peptide corresponding to amino acids 1 to 19 of SEQ ID NO: 9;
(ii) the envelope-binding protein comprises or consists of the sequence set forth in any one of SEQ ID NOs: 7 to 18; and/or
(iii) the envelope-binding protein comprises or consists of a sequence expressed from or encoded by a nucleic acid comprising the sequence set forth in any one of SEQ ID NOs: 19 to 30.

43-46. (canceled)

47. The envelope-binding protein of claim 1, for use in production of an enveloped virus in a cell culture or for use in purification of an enveloped virus from a cell culture, wherein the cell culture comprises a cell line cultured in a cell culture medium.

48-49. (canceled)

50. A method of producing an enveloped virus in a cell culture, the method comprising culturing a cell line in a cell culture medium comprising a protein that specifically binds vesicular stomatitis virus G (VSV-G) pseudotyped virus.

51. The method of claim 50, wherein:
(i) the protein comprises one or more low-density lipoprotein receptor (LDLR) domains;
(ii) the protein is the envelope-binding protein of claim 1;
(iii) the cell line is a stable producer cell line;
(iv) the cell line expresses the protein;
(v) the protein is added to the cell culture medium, wherein the protein is added to the cell culture medium at a concentration of at least 0.001 μg/mL, wherein the protein is added to the cell culture medium on each day of the cell culture; and/or
(vi) the method results in an increase in viral infectious titer yield by at least 1-fold compared to culturing in a cell culture medium in the absence of the protein.

52-58. (canceled)

59. A method of purifying an enveloped virus from a cell culture, the method comprising:
(A) (i) culturing a cell line in a cell culture medium comprising a protein that specifically binds vesicular stomatitis virus G (VSV-G) pseudotyped virus and (ii) isolating the enveloped virus from the cell culture, wherein the protein is bound to the enveloped virus, wherein the protein is the envelope-binding protein of claim 1 and wherein the method comprises affinity capture chromatography, wherein the affinity capture chromatography is protein A chromatography; or
(B) (i) culturing a cell line in a cell culture medium, (ii) harvesting the cell culture medium comprising the enveloped virus, (iii) loading the harvested cell culture medium comprising the enveloped virus onto an affinity chromatography resin comprising an envelope-binding protein of claim 1 immobilized to a matrix of the affinity chromatography resin and (iv) collecting the enveloped virus.

60-64. (canceled)

65. The method of claim 59, wherein:
(i) the enveloped virus is loaded onto an affinity capture chromatography column in a loading buffer comprising calcium, wherein the calcium is at a concentration of at least 0.01 mM,
(ii) the enveloped virus is loaded onto an affinity capture chromatography column and is eluted with a buffer comprising less than 0.01 mM calcium;
(iii) purifying the enveloped virus further comprises one or more steps selected from the group consisting of clarification filtration, anion exchange chromatography, concentration and diafiltration; and/or
(iv) the method further comprises formulating the purified enveloped virus into a pharmaceutical formulation or into a solution suitable for infecting a cell.

66-69. (canceled)

70. The method of claim 50, wherein the enveloped virus is a VSV-G-pseudotyped virus or a retrovirus.

71-72. (canceled)
